(19) United States
(12) Patent Application Publication
(10) Pub. No.: US 2007/0244158 A1
(43) Pub. Date: Oct. 18, 2007

(54) Piperdine Compound and Process for Preparing the Same

(76) Inventors: Tsutomu Miyake, Osaka (JP); Takeshi Yamanaka, Osaka (JP); Rie Kino, Osaka (JP); Hidetoshi Asai, Osaka (JP)

Correspondence Address: BROWDY AND NEIMARK, P.L.L.C.;624 NINTH STREET, NW, SUITE 300, WASHINGTON, DC, 20001-5303, US

(21) Appl. No.: 11/660,046

(22) PCT Filed: Sep. 16, 2005
(86) PCT No.: PCT/JP05/17555

§ 371 Date: Feb. 12, 2007

(30) Foreign Application Priority Data
Sep. 17, 2004 (JP) 2004-271221
May 30, 2005 (JP) 2005-157422

(51) Int. Cl. A61K 31/445; A61K 31/4545; A61P 43/00; C07D 211/40; C07D 401/04; C07D 401/06

(52) U.S. Cl. 514/317; 514/327; 546/192; 546/216

(57) ABSTRACT

The present invention is to provide a piperidine compound represented by the formula [I]: 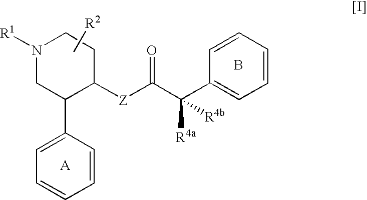 wherein Ring A is an optionally substituted benzene ring, Ring B is an optionally substituted benzene ring,

R¹ is hydrogen atom or a substituent for amino group,

R² is hydrogen atom, an optionally substituted hydroxyl group, an optionally substituted amino group, an optionally substituted alkyl group, a substituted carbonyl group or a halogen atom,

Z is oxygen atom or —N(R³)—,

R³ is hydrogen atom or an optionally substituted alkyl group,

R^{4a} and R^{4b} may be the same or different, and each is hydrogen atom or an optionally substituted alkyl group, or may be bonded to each other at the both ends to form an alkylene group, or a pharmaceutically acceptable salt thereof, which has an excellent tachykinin receptor antagonistic action.

### TECHNICAL FIELD

The present invention relates to a piperidine compound having an excellent activity of tachykinin receptor antagonist, and a process for preparing the piperidine compound.

### BACKGROUND ART

Tachykinin is a general name for a group of neuropeptides, and there have been known substance P (hereinafter referred to as “SP”), neurokinin-A, and neurokinin-B in mammals. These peptides are known to exhibit various kinds of biological activities by binding their corresponding receptors which exist in vivo (neurokinin-1, neurokinin-2, neurokinin-3). Among them, SP is one of those which have been studied the longest and in detail. Its existence was confirmed in an extract of horse intestinal tube in 1931, and it was a peptide comprising 11 amino acids, whose structure was determined in 1971.

SP exists widely in central and peripheral nervous systems, and it has physiological activities such as vasodilative action, vascular permeability promoting action, smooth muscle contracting action, neuronal excitatory action, salivary action, diuretic action, immunological action, etc., as well as a function of neurotransmitter of the primary sensory neuron. Especially, it is known that SP released from the terminal of posterior horn of spinal cord upon pain impulse transfers pain information to the secondary sensory neuron, and that SP released from the peripheral terminus induces an inflammatory response via its receptors. From these facts, SP is considered to be involved in various diseases (for example, pain, inflammation, allergy, pollakiuria, urinary incontinence, respiratory disease, mental disorder, depression, anxiety, emesis, etc.), and also, SP is considered to be involved in Alzheimer-type dementia [Review: Physiological Reviews, vol. 73, pp. 229-308 (1993), Journal of Autonomic Pharmacology, vol. 13, pp. 23-93 (1993)].
[Non-Patent publication 1] Physiological Reviews, vol. 73, pp. 229-308 (1993)
[Non-Patent publication 2] Journal of Autonomic Pharmacology, vol. 13, pp. 23-93 (1993)

### SUMMARY OF THE INVENTION

Currently, as a therapeutic agent for the above-mentioned various diseases (especially for emesis, depression, urinary disorder, etc.), there have not been discovered yet any compound having an excellent tachykinin receptor antagonistic action (specifically, SP receptor antagonistic action), and having sufficiently satisfying safety and sustainability (metabolism, dynamics in vivo, and absorption), etc. Therefore, a compound has been sought for which has an excellent tachykinin receptor antagonistic action, and has sufficiently satisfying clinical effect as the therapeutic agent.

Accordingly, an object of the present invention is to provide a compound having excellent tachykinin receptor antagonistic action, and having a clinical satisfying effect in terms of safety, sustainability (metabolism, dynamics in vivo and absorption), etc.

The present invention relates to a piperidine compound represented by the formula [I]: 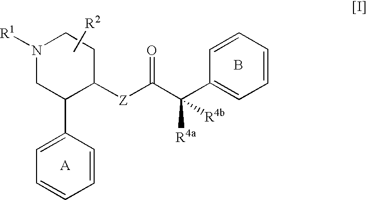
wherein Ring A represents an optionally substituted benzene ring,
Ring B represents an optionally substituted benzene ring,
R¹ represents hydrogen atom or a substituent for amino group,
R² represents hydrogen atom, an optionally substituted hydroxyl group, an optionally substituted amino group, an optionally substituted alkyl group, a substituted carbonyl group or a halogen atom,
Z represents oxygen atom or a group represented by the formula: —N(R³)—,
R³ represents hydrogen atom or an optionally substituted alkyl group,
R^{4a} and R^{4b} are the same or different from each other and each is hydrogen atom or an optionally substituted alkyl group, or may be bonded to each other at the both ends to form an alkylene group,
   or a pharmaceutically acceptable salt thereof.

### BEST MODE FOR CARRYING OUT THE INVENTION

In the present invention, Ring A represents an optionally substituted benzene ring, and a substituent of the benzene ring is exemplified by an optionally substituted alkyl group, a halogen atom, cyano group, hydroxyl group which may be protected or an alkoxy group. Ring A may have 1 to 3 of these substituent(s) which are the same or different.

In the present invention, Ring B represents an optionally substituted benzene ring, and a substituent of the benzene ring is exemplified by a haloalkyl group, a halogen atom, cyano group, phenyl group, a heterocyclic group having 1 to 4 atoms selected from nitrogen atom, oxygen atom and sulfur atom as hetero atom(s), an alkyl group, hydroxyl group which may be protected or an alkoxy group. Ring B may have 1 to 3 of these substituent(s) which are the same or different.

A preferred example of Ring A and Ring B in the compound of the present invention is exemplified by a compound wherein Ring A is a benzene ring of the formula: 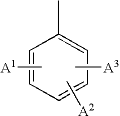
and Ring B is a benzene ring of the formula: 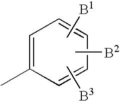
wherein A¹, A² and A³ are the same or different, and each is hydrogen atom, a halogen atom, an optionally substituted alkyl group, hydroxyl group which may be protected or an alkoxy group, B¹, B² and B³ are the same or different, and each is hydrogen atom, a haloalkyl group, a halogen atom, cyano group, phenyl group, a heterocyclic group having 1 to 4 atoms selected from nitrogen atom, oxygen atom and sulfur atom as hetero atom(s), an alkyl group, hydroxyl group which may be protected or an alkoxy group. The substituent for the optionally substituted alkyl group is exemplified by a halogen atom, etc. The haloalkyl group is exemplified by an alkyl group substituted by 1 to 3 halogen atoms which may be the same or different from each other, and specifically mentioned a trihalogenoalkyl group. The trihalogenoalkyl group is exemplified by trifluoromethyl group or trichloromethyl group, etc. The heterocyclic group having 1 to 4 atoms selected from nitrogen atom, oxygen atom and sulfur atom as hetero atom(s) is exemplified by tetrazolyl group.

In the present invention, the protective group for the optionally protected hydroxyl group is exemplified by a conventionally used protective group such as an optionally substituted arylalkyl group, an optionally substituted silyl group, an acyl group, etc. Of these, preferred is exemplified by an arylalkyl group such as benzyl group, phenethyl group, etc., a substituted silyl group such as tert-butyldimethylsilyl group, tert-butyldiphenylsilyl group, etc., an acyl group such as formyl group, acetyl group, propionyl group, malonyl group, acryloyl group, benzoyl group, etc.

In the present invention, R¹ represents hydrogen atom or a substituent for amino group, and the substituent of the amino group in R¹ is exemplified by an optionally substituted alkyl group, an optionally substituted cycloalkyl group, an optionally substituted aryl group, an optionally substituted amino group, a substituted carbonyl group, a substituted sulfonyl group or an optionally substituted heterocyclic group.

Of these, R¹ is preferably an optionally substituted alkyl group, an optionally substituted carbonyl group or an optionally substituted heterocyclic group, and R¹ is further preferable a substituted carbonyl group or an optionally substituted heterocyclic group.

In the present invention, the substituent of the optionally substituted alkyl group of R¹ is exemplified by an alkoxy group, a halogen atom, an alkoxycarbonyl group, morpholinocarbonyl group, a dialkylaminocarbonyl group, an optionally substituted heterocyclic group, hydroxyl group, a hydroxyalkylaminocarbonyloxy group or an alkylpiperazinocarbonyl group. The substituent of the optionally substituted heterocyclic group is exemplified by an alkanoyl group optionally substituted by hydroxyl group, or oxo group. The substituent(s) for the heterocyclic group may be the same or different from each other, and the number thereof may be 1 or 2. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

In the present invention, the substituent of the optionally substituted cycloalkyl group of R¹ is exemplified by an alkyl group, hydroxyl group, etc.

In the present invention, the substituent of the optionally substituted aryl group of R¹ is exemplified by hydroxyl group, an alkyl group, cyano group, a halogen atom, etc. The aryl group is exemplified by phenyl group, naphthyl group, anthracenyl group or phenanthrenyl group.

In the present invention, the substituent of the optionally substituted amino group of R¹ is exemplified by
(1) an optionally substituted alkyl group,
(2) an optionally substituted cycloalkyl group,
(3) an optionally substituted aryl group or
(4) a heterocyclic group having 1 to 4 atoms selected from nitrogen atom, oxygen atom and sulfur atom as hetero atom(s), and the heterocyclic group may have a substituent(s).

The substituent of the optionally substituted alkyl group in the above-mentioned (1) is exemplified by hydroxyl group, a dialkylaminocarbonyl group, an alkoxy group, a dialkylamino group, cyano group, morpholino group, pyridyl group or a halogen atom.

The substituent of the substituted cycloalkyl group of the above-mentioned (2) is exemplified by hydroxyl group, an alkyl group, cyano group, a halogen atom, etc.

The substituent of the optionally substituted aryl group of substituent the above-mentioned (3) is exemplified by hydroxyl group, an alkyl group, cyano group, a halogen atom, etc. The aryl group is exemplified by phenyl group, naphthyl group, anthracenyl group or phenanthrenyl group.

The heterocyclic group having 1 to 4 atoms selected from nitrogen atom, oxygen atom and sulfur atom as hetero atom(s) of the above-mentioned (4) is exemplified by a saturated or unsaturated monocyclic or bicyclic heteroaromatic group, and may include, for example, thienyl group, furyl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. Of these heterocyclic groups, suitably used are pyridyl group, pyrrolyl group, piperazinyl group, quinolyl group, piperidinyl group, pyrimidinyl group, thiazolyl group, pyrazinyl group, morpholino group, indolyl group, cinnolinyl group, furyl group, thienyl group, pyrrolidinyl group, imidazolidinyl group, etc. The substituent of the heterocyclic group is exemplified by a dialkylamino group, an alkoxycarbonyl group, an alkyl group, an alkoxy group, oxo group, hydroxyl group or a halogen atom.

In the present invention, the substituent of the substituted carbonyl group of R¹ is exemplified by
(1) an optionally substituted alkyl group,
(2) an optionally substituted cycloalkyl group,
(3) an optionally substituted aryl group,
(4) an optionally substituted heterocyclic group,
(5) an optionally substituted amino group or
(6) an optionally substituted alkoxy group.

The substituent of the optionally substituted alkyl group of the above-mentioned (1) is exemplified by
(I) hydroxyl group,
(II) a substituted carbonylamino group,
(III) an optionally substituted aminocarbonyl group,
(IV) an alkylsulfonyl group,
(V) a heterocyclic group or
(VI) nitro group.

The substituent of the substituted carbonylamino group of the above-mentioned (II) is exemplified by (i) hydroxyl group, (ii) an optionally substituted alkyl group or (iii) an optionally substituted heterocyclic group, etc. The substituent of the optionally substituted alkyl group of the above-mentioned (ii) is exemplified by hydroxyl group or a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The substituent of the optionally substituted heterocyclic group of the above-mentioned (iii) is exemplified by an alkanoyl group optionally substituted by hydroxyl group, oxo group or hydroxyl group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted aminocarbonyl group of the above-mentioned (III) is exemplified by (i) an optionally substituted alkyl group or (ii) an optionally substituted heterocyclic group. The substituent of the optionally substituted alkyl group of the above-mentioned (i) is exemplified by hydroxyl group or a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The substituent of the optionally substituted heterocyclic group of the above-mentioned (ii) is exemplified by an alkanoyl group optionally substituted by hydroxyl group, oxo group or hydroxyl group. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The heterocyclic group of the above-mentioned (V) is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, tetrazolyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, tetrahydropyranyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent of the optionally substituted cycloalkyl group of the above-mentioned (2) is exemplified by an optionally substituted hydroxyl group, an alkyl group, cyano group, a halogen atom, etc. The cycloalkyl group may have 1 or 2 substituent(s). The substituent(s) for the optionally substituted hydroxyl group is exemplified by an alkyl group optionally substituted by hydroxyl group, etc.

The substituent of the optionally substituted aryl group of the above-mentioned (3) is exemplified by hydroxyl group, an alkyl group, cyano group, a halogen atom, etc. The aryl group is exemplified by phenyl group, naphthyl group, anthracenyl group or phenanthrenyl group.

The substituent of the optionally substituted heterocyclic group of the above-mentioned (4) is exemplified by
(I) oxo group,
(II) an optionally substituted alkanoyl group,
(III) an optionally substituted alkyl group,
(IV) an optionally substituted hydroxyl group or
(V) an alkoxycarbonyl group.

The heterocyclic group may have 1 to 2 substituent(s) which may be the same or different from each other. The heterocyclic group is exemplified by a heteromonocyclic group having 1 to 4 atoms selected from sulfur atom, nitrogen atom and oxygen atom as hetero atom(s), and a saturated heteromonocyclic group is preferably used. The heteromonocyclic group is exemplified by pyrazinyl group, piperidinyl group, piperazinyl group, pyridyl group, tetrazolidyl group, pyrrolidinyl group, imidazolidinyl group, morpholinyl group, thiomorpholinyl group, tetrahydropyranyl group, tetrahydrothiopyranyl group, azetidinyl group or thietanyl group. Of these, pyrazinyl group, piperidinyl group, piperazinyl group, pyrrolidinyl group, morpholinyl group, thiomorpholinyl group, tetrahydropyranyl group, tetrahydrothiopyranyl group or azetidinyl group is preferred.

The substituent of the optionally substituted alkanoyl group of the above-mentioned (II) is exemplified by hydroxyl group, etc.

The substituent of the optionally substituted alkyl group of the above-mentioned (III) is exemplified by hydroxyl group or a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc.

The substituent(s) for the optionally substituted hydroxyl group of the above-mentioned (IV) is exemplified by an alkyl group optionally substituted by hydroxyl group, etc.

The substituent of the optionally substituted amino group of the above-mentioned (5) is exemplified by an alkyl group optionally substituted by hydroxyl group, a heterocyclic group having 1 to 4 atoms selected from nitrogen atom, oxygen atom and sulfur atom as hetero atom(s), and the heterocyclic group may have a substituent(s). The substituent of the heterocyclic group is exemplified by oxo group, hydroxyl group, an alkanoyl group or an alkyl group. The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. The amino group may have 1 to 2 substituent(s).

The substituent of the optionally substituted alkoxy group of the above-mentioned (6) is exemplified by hydroxyl group.

In the present invention, the substituent of the substituted sulfonyl group of R¹ is an optionally substituted alkyl group. The substituent of the optionally substituted alkyl group is exemplified by hydroxyl group or an alkanoyloxy group.

In the present invention, the substituent of the optionally substituted heterocyclic group of R¹ is exemplified by
(I) an optionally substituted alkanoyl group,
(II) a substituted carbonyl group,
(III) an optionally substituted alkylsulfonyl group,
(IV) an optionally substituted alkyl group,
(V) hydroxyl group or
(VI) oxo group.

The heterocyclic group may have 1 to 2 substituent(s) which may be the same or different from each other. The heterocyclic group is exemplified by a heterocyclic group having 1 to 4 atoms selected from nitrogen atom, oxygen atom and sulfur atom as hetero atom(s). The heterocyclic group is exemplified by a saturated or unsaturated monocyclic or bicyclic heteroaromatic group, and may include, for example, thienyl group, furyl group, tetrahydrofuryl group, pyranyl group, pyrrolyl group, imidazolyl group, pyrazolyl group, isothiazolyl group, isoxazolyl group, pyridyl group, pyrazinyl group, pyrimidinyl group, pyridazinyl group, azetidinyl group, thietanyl group, pyrrolidinyl group, pyrrolinyl group, imidazolidinyl group, imidazolinyl group, pyrazolidinyl group, pyrazolinyl group, piperidinyl group, piperazinyl group, morpholinyl group, thiomorpholinyl group, tetrahydropyranyl group, tetrahydrothiopyranyl group, benzothienyl group, benzofuryl group, isobenzofuranyl group, chromenyl group, indolyl group, isoindolyl group, indazolyl group, purinyl group, quinolizinyl group, naphthyridinyl group, quinoxalinyl group, cinnolinyl group, quinolyl group, isoquinolyl group, benzothiazolyl group, benzisothiazolyl group, quinazolinyl group, phthalazinyl group, benzoxazolyl group, benzimidazolyl group, pteridinyl group, pyridopyrimidinyl group, isochromanyl group, chromanyl group, indolinyl group, isoindolinyl group, tetrahydroquinolyl group, tetrahydroisoquinolyl group, tetrahydroquinoxalinyl group, dihydrophthalazinyl group, etc. Of these heterocyclic groups, piperidinyl group, tetrahydropyranyl group, tetrahydrothiopyranyl group, azetidinyl group or thietanyl group is suitably used.

The substituent of the optionally substituted alkanoyl group of the above-mentioned (I) is exemplified by hydroxyl group, an alkoxy group, a phenylalkoxy group, an alkanoylamino group, an alkylsulfonyl group, an alkanoyl group, aminocarbonyl group, etc.

The substituent of the substituted carbonyl group of the above-mentioned (II) is exemplified by phenyl group, a hydroxycycloalkyl group, a dialkylamino group, a hydroxyalkylamino group, amino group, tetrahydrofuryl group, an alkanoyl group, aminocarbonyl group, or a pyrrolidinyl group which is optionally substituted by 1 or 2 substituent(s) selected by oxo group and an alkyl group, etc.

The substituent of the optionally substituted alkylsulfonyl group of the above-mentioned (III) is exemplified by hydroxyl group, amino group, heterocyclic group, etc.

The substituent of the optionally substituted alkyl group of the above-mentioned (IV) is exemplified by an alkylsulfonyl group, carboxyl group, etc.

In the present invention, R² is hydrogen atom, an optionally substituted hydroxyl group, an optionally substituted amino group, an optionally substituted alkyl group, a substituted carbonyl group or a halogen atom.

In the present invention, the substituent of the optionally substituted hydroxyl group of R² is exemplified by an alkyl group optionally substituted by hydroxyl group.

In the present invention, the substituent of the optionally substituted amino group of R² is exemplified by an alkyl group optionally substituted by hydroxyl group.

In the present invention, the substituent of the optionally substituted alkyl group of R² is an alkoxy group optionally substituted by hydroxyl group, or hydroxyl group.

In the present invention, the substituent of the substituted carbonyl group of R² is exemplified by hydroxyl group, an alkoxy group optionally substituted by hydroxyl group or an alkylamino group optionally substituted by hydroxyl group.

In the present invention, Z is exemplified by oxygen atom or a group represented by —N(R³)—.

In the present invention, R³ is exemplified by hydrogen atom or an optionally substituted alkyl group. The substituent of the optionally substituted alkyl group of R³ is exemplified by hydroxyl group, an alkanoyl group, a halogen atom, an alkoxy group or an alkylamino group.

In the present invention, R^{4a} and R^{4b} may be the same or different from each other, and are each exemplified by hydrogen atom, an optionally substituted alkyl group, or may be bonded to each other at the both ends to form an alkylene group. The substituent of the optionally substituted alkyl group is exemplified by hydroxyl group, etc.

As the preferred compound of the present invention, a compound where R¹ is an optionally substituted alkyl group is mentioned. The substituent of the alkyl group is preferably exemplified by an alkoxy group, a halogen atom, a dialkylaminocarbonyl group, oxopyridyl group, dioxopyrrolidinyl group, morpholinocarbonyl group, hydroxyl group, an alkoxycarbonyl group or a hydroxyalkylaminocarbonyloxy group, more preferably an alkoxy group, a halogen atom, a dialkylaminocarbonyl group, oxopyridyl group or dioxopyrrolidinyl group.

As the preferred compound of the present invention, a compound where R¹ is a substituted carbonyl group is mentioned. The preferred substituent of the carbonyl group is exemplified by an alkyl group optionally substituted by hydroxyl group, an alkanoylamino group optionally substituted by an alkyl group, an alkylsulfonyl group, tetrahydropyranyl group, tetrazolyl group or nitro group; an alkoxy group; a hydroxyalkylamino group; a cycloalkyl group substituted by 1 or 2 substituents selected from hydroxyl group and an alkyl group; piperidinyl group substituted by 1 or 2 substituents selected from an alkanoyl group, an alkoxycarbonyl group, oxo group and an alkyl group; tetrahydropyranyl group; tetrahydrothiopyranyl group the sulfur atom of which is substituted by 2 oxo groups; pyrrolidinyl group substituted by 1 or 2 substituents selected from an alkanoyl group, hydroxyl group, an alkyl group and oxo group; pyrazinyl group; morpholino group; thiomorpholino group the sulfur atom of which is optionally substituted by oxo group; or piperazine group substituted by an alkyl group which may be optionally substituted by hydroxy group, or by an alkanoyl group.

As the preferred compound of the present invention, a compound where R¹ is a substituted sulfonyl group is mentioned. The substituent of the sulfonyl group is preferably exemplified by an alkyl group.

As the preferred compound of the present invention, a compound where R¹ is an optionally substituted heterocyclic group is mentioned. The heterocyclic group is preferably exemplified by piperidinyl group, tetrahydropyranyl group, tetrahydrothiopyranyl group, thietanyl group or azetidinyl group. Also, the substituent of the heterocyclic group is preferably exemplified by an alkanoyl group, a hydroxyalkanoyl group, a dihydroxyalkanoyl group, an alkoxyalkanoyl group, an alkanoylaminoalkanoyl group, an alkylsulfonylalkanoyl group, an alkanoylalkanoyl group, an aminocarbonylalkanoyl group, an alkoxycarbonyl group, an alkylsulfonyl group, oxo group, a phenylalkoxycarbonyl group, a dialkylcarbonyl group, a hydroxycycloalkyl group, a hydroxyalkylaminocarbonyl group, aminocarbonyl group, tetrahydrofurylcarbonyl group, an alkyldiketonyl group, an aminodiketonyl group, an alkylsulfonylalkyl group, an carboxyalkyl group, or a pyrrolidinylcarbonyl group which is optionally substituted by substituent(s) selected by oxo group and an alkyl group, etc. The heterocyclic group may have 1 or 2 substituents which may be the same or different from each other.

As the compound [I] of the present invention, a compound where Ring A is a benzene ring represented by the formula: 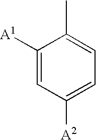
Ring B is a benzene ring represented by the formula: 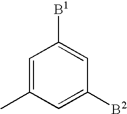
A¹ is hydrogen atom, a halogen atom, an alkyl group or an alkoxy group, A² is hydrogen atom or a halogen atom, B¹ is hydrogen atom, an alkyl group, a halogen atom, cyano group, an alkoxy group or a trihalogenoalkyl group, B² is hydrogen atom, an alkyl group, a halogen atom, cyano group, an alkoxy group or a trihalogenoalkyl group, R¹ is hydrogen atom; an alkyl group substituted by an alkoxy group, a halogen atom, a dialkylaminocarbonyl group, oxopyridyl group, dioxopyrrolidinyl group, morpholinocarbonyl group, hydroxyl group, an alkoxycarbonyl group, morpholinoaminocarbonyl group, a hydroxyalkylaminocarbonyloxy group or an alkylpiperazinocarbonyl group; a hydroxycycloalkyl group; carboxyl group; an alkanoyl group substituted by hydroxyl group, an alkanoylamino group optionally substituted by an alkyl group, an alkylsulfonyl group, tetrahydropyranyl group, tetrazolyl group or nitro group; an alkoxycarbonyl group optionally substituted by hydroxyl group; pyrimidinylaminocarbonyl group; an alkylaminocarbonyl group the alkyl group portion of which is optionally substituted by hydroxyl group or cyano group; a di(hydroxyalkyl)aminocarbonyl group; a cycloalkylcarbonyl group substituted by 1 or 2 groups selected from hydroxyl group and an alkyl group; pyridylaminocarbonyl group the pyridyl group portion of which is substituted by hydroxyl group; an aminocarbonyl group substituted by an alkylpyrido group and an alkyl group; piperidinylcarbonyl group substituted by 1 or 2 groups selected from an alkanoyl group, an alkoxycarbonyl group, oxo group and an alkyl group; tetrahydropyranylcarbonyl group; tetrahydrothiopyranylcarbonyl group the sulfur atom of which is substituted by 2 oxo groups; piperazinocarbonyl group substituted by oxo group, an alkyl group optionally substituted by hydroxyl group, pyrimidinyl group, an alkylsulfonyl group or an alkanoyl group; pyradinylcarbonyl group; morpholinocarbonyl group; thiomorpholinocarbonyl group the sulfur atom of which is optionally substituted by oxo group; pyrrolidinylcarbonyl group substituted by 1 or 2 groups selected from an alkanoyl group, hydroxyl group, an alkyl group and oxo group; an alkylsulfonyl group optionally substituted by hydroxyl group or an alkanoyloxy group; piperidinyl group substituted by an alkanoyl group, an alkoxycarbonyl group or an alkylsulfonyl group; tetrahydropyranyl group; tetrahydrothiopyranyl group the sulfur atom of which is optionally substituted by 1 or 2 oxo groups; thietanyl group the sulfur atom of which is optionally substituted by 2 oxo groups; or azetidinyl group optionally substituted by a phenylalkoxycarbonyl group, an alkanoyl group, a hydroxyalkanoyl group, an alkoxycarbonyl group, a dihydroxyalkanoyl group, an alkoxyalkanoyl group, an alkanoylaminoalkanoyl group, an alkylsulfonylalkanoyl group, an alkanoylalkanoyl group, an aminocarbonylalkanoyl group, a hydroxyalkoxycarbonyl group, an alkylsulfonyl group, a dialkylaminocarbonyl group, a hydroxyalkylcarbonyl group, aminocarbonyl group, a hydroxycycloalkylcarbonyl group, tetrahydrofurylcarbonyl group, an alkyldiketonyl group, an aminodiketonyl group, an alkylsulfonylalkyl group, a carboxylalkyl group or a pyrrolidinyl group which is optionally substituted by 1 or 2 substituent(s) selected by oxo group and an alkyl group, R² is hydrogen atom, Z is oxygen atom or a group represented by —N(R³)—, R³ is an alkyl group optionally substituted by hydroxyl group, R^{4a} is hydrogen atom or an alkyl group optionally substituted by hydroxyl group, and R^{4b} is hydrogen atom or an alkyl group optionally substituted by hydroxyl group is mentioned.

Of these, preferred are compounds wherein Ring A is a benzene ring represented by the formula: 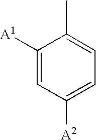
Ring B is a benzene ring represented by the formula: 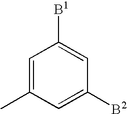
A¹ is hydrogen atom, an alkyl group or a halogen atom, A² is hydrogen atom or a halogen atom, B¹ is a trihalogenomethyl group, a halogen atom or an alkyl group, B² is a trihalogenomethyl group, a halogen atom or an alkyl group, R¹ is hydrogen atom; an alkyl group substituted by an alkoxy group, a halogen atom, a dialkylaminocarbonyl group, oxopyridyl group or dioxopyrrolidinyl group; an alkanoyl group substituted by hydroxyl group, an alkanoylamino group optionally substituted by an alkyl group, an alkylsulfonyl group, tetrahydropyranyl group, tetrazolyl group or nitro group; an alkoxycarbonyl group; a hydroxyalkylaminocarbonyl group; a cycloalkylcarbonyl group substituted by 1 or 2 groups selected from hydroxyl group and an alkyl group; piperidinylcarbonyl group substituted by 1 or 2 groups selected from an alkanoyl group, an alkoxycarbonyl group, oxo group and an alkyl group; tetrahydropyranylcarbonyl group; tetrahydrothiopyranylcarbonyl group the sulfur atom of which is substituted by 2 oxo groups; pyrrolidinylcarbonyl group substituted by 1 or 2 groups selected from an alkanoyl group, hydroxyl group, an alkyl group and oxo group; pyradinylcarbonyl group; morpholinocarbonyl group; thiomorpholinocarbonyl group the sulfur atom of which is optionally substituted by oxo group; piperazinocarbonyl group substituted by an alkyl group optionally substituted by hydroxyl group, or by an alkanoyl group; piperidinyl group substituted by an alkanoyl group, an alkoxycarbonyl group or an alkylsulfonyl group; tetrahydropyranyl group; tetrahydrothiopyranyl group the sulfur atom of which is optionally substituted by 1 or 2 oxo groups; thietanyl group the sulfur atom of which is optionally substituted by 2 oxo groups; or azetidinyl group optionally substituted by a phenylalkoxycarbonyl group, an alkanoyl group, a hydroxyalkanoyl group, a dihydroxyalkanoyl group, an alkoxyalkanoyl group, an alkanoylaminoalkanoyl group, an alkylsulfonylalkanoyl group, an alkanoylalkanoyl group, an aminocarbonylalkanoyl group, an alkoxycarbonyl group, a hydroxyalkoxycarbonyl group, an alkylsulfonyl group, a dialkylaminocarbonyl group, a hydroxyalkylaminocarbonyl group, aminocarbonyl group, an hydroxycycloalkylcarbonyl group, tetrahydrofurylcarbonyl group, an alkyldiketonyl group, an aminodiketonyl group, an alkylsulfonylalkyl group, a carboxylalkyl group or a pyrrolidinyl group which is optionally substituted by 1 or 2 substituent(s) selected by oxo group and an alkyl group, R² is hydrogen atom, Z is a group represented by —N(R³)—, R³ is an alkyl group, R^{4a} is hydrogen atom or an alkyl group, and R^{4b} is hydrogen atom or an alkyl group.

Moreover, preferred are compounds wherein Ring A is a benzene ring represented by the formula: 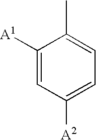
Ring B is a benzene ring represented by the formula: 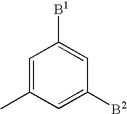
A¹ is hydrogen atom or an alkyl group, A² is a halogen atom, B¹ is a trihalogenomethyl group, B² is a trihalogenomethyl group, R¹ is an alkanoylaminoalkanoyl group; piperidinylcarbonyl group optionally substituted by 1 or 2 group(s) selected from an alkanoyl group, oxo group and an alkyl group; piperidinyl group substituted by an alkanoyl group; tetrahydrothiopyranyl group the sulfur atom of which is di-substituted by oxo group; thietanyl group the sulfur atom of which is optionally substituted by 2 oxo groups; or azetidinyl group substituted by an alkanoyl group optionally substituted by hydroxyl group, an alkoxycarbonyl group, an alkylsulfonyl group or dialkylaminocarbonyl group, R² is hydrogen atom, Z is a group represented by the formula —N(R³)—, R³ is an alkyl group, R^{4a} is hydrogen atom or an alkyl group, R^{4b} is hydrogen atom or an alkyl group.

Furthermore, in the compounds of the present invention, preferred compounds are a compound selected from the following (A) to (S) or a pharmaceutically acceptable salt thereof.
(A) (3S,4S)-3-(4-Fluoro-2-methylphenyl)-4-{N-methyl-2-(3,5-bis-trifluoromethylphenyl)isobutyrylamino}-1-(tetrahydrothiopyran-1,1-dioxid-4-yl)piperidine,
(B) (3S,4S)-1-(Acetylpiperidin-4-yl)-3-(4-fluoro-2-methylphenyl)-4-{N-methyl-2-(3,5-bistrifluoromethylphenyl)isobutyrylamino}piperidine,
(C) (3R,4R)-1-(Acetylpiperidin-4-yl)-3-(4-fluoro-2-methylphenyl)-4-{N-methyl-2-(3,5-bistrifluoromethylphenyl)isobutyrylamino}piperidine,
(D) (3S,4S)-3-(4-Fluoro-2-methylphenyl)-4-{N-methyl-2-(3,5-bistrifluoromethylphenyl)isobutyrylamino}-1-(thiethan-3-yl)piperidine,
(E) (3S,4S)-1-(1,1-Dioxothiethan-3-yl)-3-(4-fluoro-2-methylphenyl)-4-{N-methyl-2-(3,5-bistrifluoromethylphenyl)isobutyrylamino}piperidine,
(F) (3S,4S)-1-(1-Acetylazetidin-3-yl)-3-(4-fluoro-2-methylphenyl)-4-{N-methyl-2-(3,5-bistrifluoromethylphenyl)isobutyrylamino}piperidine,
(G) (3S,4S)-3-(4-Fluoro-2-methylphenyl)-1-(1-propionylazetidin-3-yl)-4-{N-methyl-2-(3,5-bistrifluoromethylphenyl)isobutyrylamino}-piperidine,
(H) (3R,4R)-3-(4-Fluoro-2-methylphenyl)-1-(1-propionylazetidin-3-yl)-4-{N-methyl-2-(3,5-bistrifluoromethylphenyl)isobutyrylamino}-piperidine,
(I) (3S,4S)-3-(4-Fluoro-2-methylphenyl)-1-(1-hydroxyacetylazetidin-3-yl)-4-{N-methyl-2-(3,5-bistrifluoromethylphenyl)isobutyrylamino}-piperidine,
(J) (3R,4R)-3-(4-Fluoro-2-methylphenyl)-1-(1-hydroxyacetylazetidin-3-yl)-4-{N-methyl-2-(3,5-bistrifluoromethylphenyl)isobutyrylamino}-piperidine,
(K) (3S,4S)-3-(4-Fluoro-2-methylphenyl)-4-{N-methyl-2-(3,5-bistrifluoromethylphenyl)isobutyrylamino}-1-(2-methylpropionylazetidin-3-yl)piperidine,
(L) (3R,4R)-3-(4-Fluoro-2-methylphenyl)-4-{N-methyl-2-(3,5-bistrifluoromethylphenyl)isobutyrylamino)-1-(2-methylpropionylazetidin-3-yl}piperidine,
(M) (3S,4S)-3-(4-Fluoro-2-methylphenyl)-1-(methoxycarbonylazetidin-3-yl)-4-{N-methyl-2-(3,5-bistrifluoromethylphenyl)isobutyrylamino}-piperidine,
(N) (3S,4S)-1-(2-Acetylaminoacetyl)-3-(4-fluoro-2-methylphenyl)-4-{N-methyl-2-(3,5-bistrifluoromethylphenyl)isobutyrylamino}piperidine,
(O) (3S,4S)-3-(4-Fluoro-2-methylphenyl)-1-(methanesulfonylazetidin-3-yl)-4-{N-methyl-2-(3,5-bistrifluoromethylphenyl)isobutyrylamino}-piperidine,
(P) (3R,4R)-1-(2-Acetylaminoacetyl)-3-(4-fluoro-2-methylphenyl)-4-{N-methyl-2-(3,5-bistrifluoromethylphenyl)isobutyrylamino}piperidine,
(Q) (3S,4S)-1-(Dimethylaminocarbonylazetidin-3-yl)-3-(4-fluoro-2-methylphenyl)-4-{N-methyl-2-(3,5-bistrifluoromethylphenyl)isobutyrylamino}piperidine,
(R) (3R,4R)-1-(Dimethylaminocarbonylazetidin-3-yl)-3-(4-fluoro-2-methylphenyl)-4-{N-methyl-2-(3,5-bistrifluoromethylphenyl)isobutyrylamino)piperidine, and
(S) (3S,4S)-3-(4-Fluoro-2-methylphenyl)-1-((S)-1-methyl-6-oxo-piperidin-2-yl)-4-{N-methyl-2-(3,5-bistrifluoromethylphenyl)isobutyrylamino}piperidine.

The compound [I] of the present invention can be used for a pharmaceutical use either in a free form or in form of a pharmaceutically acceptable salt.

As the pharmaceutically acceptable salt of the compound [I] of the present invention, there may be mentioned, for example, an inorganic acid salt such as hydrochloride, sulfate, phosphate and hydrobromide; and an organic acid salt such as acetate, fumarate, oxalate, citrate, methanesulfonate, benzenesulfonate, tosylate, maleate,.succinate and tartarate.

Further, the compound [I] of the present invention or a pharmaceutically acceptable salt thereof includes any of its internal salts, solvates and hydrates, etc.

Although an optical isomer based on an asymmetric carbon can be present in the compound [I] of the present invention, the present invention includes any of these optical isomers and the mixture thereof.

The compound [I] or a pharmaceutically acceptable salt thereof of the present invention has an excellent tachykinin receptor antagonistic action, particularly an SP receptor antagonistic action, whereby it is useful as a safe medicament for prophylaxis and treatment for inflammation or allergic diseases (for example, atopic dermatitis, dermatitis, herpes, psoriasis, asthma, bronchitis, expectoration, rhinitis, rheumatoid arthritis, osteoarthritis, osteoporosis, multiple sclerosis, conjunctivitis, ophthalmia, cystitis, etc.), pain, migraine, neuralgia, itchiness, cough, and further central nervous system diseases (for example, schizophrenia, Parkinson's disease, depression, uneasiness, psychosomatic disorder, morphine dependence, dementia (for example, Alzheimer's disease, etc.), etc.), digestive organs disease (for example, irritable bowel syndrome, ulcerative colitis, Crohn's disease, disorder (for example, gastritis, gastric ulcer, etc.) related to urease-positive Spirillum (for example, helicobacter pylori, etc.), etc.), nausea, emesis, urinary disorder (for example, pollakiurea, urinary incontinence, etc.), circulatory disease (for example, angina pectoris, hypertension, cardiac failure, thrombosis, etc.) and immune disorder, etc. in mammals (for example, mouse, guinea pig, Mongolian gerbil, ferret, rat, hamster, rabbit, cat, dog, bovine, sheep, monkey, human, etc.). Particularly, since compound [I] or a pharmaceutically acceptable salt thereof which is an active ingredient of the present invention has a high penetration to the brain and has a low toxicity (high safety), showing almost no side effect, it is useful as a therapeutic or prophylactic agent for central nervous system diseases such as emesis, depression and so forth, or urinary disorder such as pollakiuria, etc.

Measurements on the compound of the present invention or a pharmaceutically acceptable salt thereof can be carried out, according to the method described in European Journal of Pharmacology, vol. 254, pages 221-227 (1994) with respect to a neurokinin-1 receptor binding action, and according to the method described in European Journal of Pharmacology, vol. 265, pages 179-183 (1994) with respect to neurokinin-1 receptor antagonstic action, further according to the method described in Journal of Urology, vol. 155, No. 1, pages 355-360 (1996) with regard to an inhibitory action on pollakiuria.

The compound [I] or a pharmaceutically acceptable salt thereof of the present invention can be administered orally or parenterally, and it can be formulated into a suitable preparation, using a conventionally used pharmaceutical carrier for an oral or parenteral administration. As such a pharmaceutical carrier, there may be mentioned, for example, a binder (syrup, Gum Arabic, gelatin, sorbitol, tragacanth, polyvinylpyrrolidone, etc.), an excipient (lactose, sugar, corn starch, potassium phosphate, sorbitol, glycine, etc.), a lubricant (magnesium stearate, talc, polyethylene glycol, silica, etc.), a disintegrator (potato starch, etc.) and a wetting agent (anhydrous lauryl sodium sulfate, etc.), and the like.

Also, when these pharmaceutical preparations are administered orally, they may be a solid preparation such as tablets, granules, capsules and powders, or a liquid preparation such as solution, suspension and emulsion. On the other hand, when they are administered parenterally, for example, they can be administered as an injection solution or an infusion solution, using distilled water for injection, physiological saline, aqueous glucose solution, etc., or they may be administered as a suppository, and the like.

A dose of the compound [I] or a pharmaceutically acceptable salt thereof of the present invention may vary depending on an administration method, an age, a body weight or a condition of a patient, etc., and, for example, in case of oral administration, it is usually administered in a dose of 0.1 to 20 mg/kg per day, and particularly preferably 0.1 to 10 mg/kg per day, and in case of parenteral administration, usually in a dose of 0.01 to 10 mg/kg per day, particularly preferably 0.01 to 1 mg/kg per day.

[Method A]

The compound of the formula [I]: 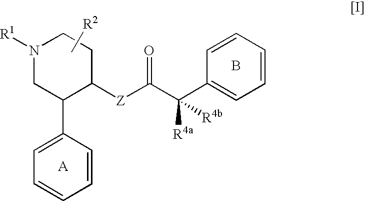
wherein Ring A represents an optionally substituted benzene ring,
Ring B represents an optionally substituted benzene ring,
R¹ represents hydrogen atom or a substituent for the amino group,
R² represents hydrogen atom, an optionally substituted hydroxyl group, an optionally substituted amino group, an optionally substituted alkyl group, a substituted carbonyl group or a halogen atom,
Z represents oxygen atom or a group represented by —N(R³)—,
R³ represents hydrogen atom or an optionally substituted alkyl group,
R^{4a} and R^{4b} may be the same or different from each other, and each is hydrogen atom or an optionally substituted alkyl group, or may be bonded to each other at the both ends to form an alkylene group,
   according to the present invention can be prepared, for example, by reacting the compound of the formula [II]: 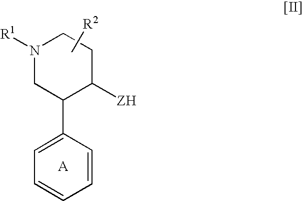
wherein Ring A, Z, R¹ and R² have the same meanings as defined above,
   with the compound of the formula [III]: 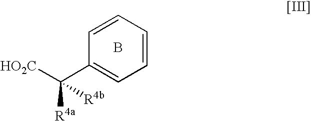
wherein Ring B, R^{4a} and R^{4b} have the same meanings as defined above.

This [Method A] can be carried out as mentioned below.

[Method A]

The reaction of Compound [II] with Compound [III] can be carried out in a solvent in the presence of a condensing agent; or reacting Compound [II] with a reactive derivative (acid halide, acid anhydride, active amide, active ester, mixed acid anhydride, etc.) of Compound [III] in a solvent in the presence or absence of a base and in the presence or absence of a condensing agent, to prepare a target compound. As the base, organic bases such as pyridine, 4-dimethylaminopyridine, N-methylmorpholine, triethylamine, N,N-dimethylaniline, N,N-diethylaniline, 1,8-diazabicyclo-[5.4.0]undec-7-ene, etc., inorganic bases such as sodium hydrogen carbonate, potassium hydrogen carbonate, sodium carbonate, potassium carbonate, etc. can be used. As the condensing agent, 1,1′-carbonyldiimidazole, 1,3-dicyclohexylcarbodiimide, 1-ethyl-3-(3-dimethylaminopropyl)carbodiimide hydrochloride, propanephosphonic acid anhydride, benzotriazol-1-yloxytris(dimethylamino)phosphonium hexafluoro phosphorus, etc. can be used. As the solvent, any solvent can be used as long as it does not exert any bad effect on the reaction, and, for example, N,N-dimethylformamide, dichloromethane, chloroform, tetrahydrofuran, dioxane, ethyl acetate, 1,3-dimethyl-2-imidazolidinone, etc. can be used. This reaction suitably proceeds, for example, at −20° C. to 60° C., particularly preferably at 5° C. to 50° C. As the active ester of Compound [III], an ester with N-hydroxysuccinic imide, N-hydroxyphthalimide, 1-hydroxybenzotriazole or p-nitrophenol can be used. As the acid halide of Compound [III], an acid chloride, an acid bromide, etc., can be suitably used. Also, as the active amide of Compound [III], an amide with imidazole, etc. can be used.

The objective Compound [I] of the present invention can be also prepared by converting the group R¹ of the compound obtained as mentioned above into the other substituent. Such a converting method of the substituent can be suitably selected depending on the kinds of the substituents to be converted, for example, it can be carried out by the following (Method a) to (Method i).
(Method a): The objective Compound [I] in which the group R¹ in the formula [I] is hydrogen atom can be prepared by eliminating a protective group from a corresponding Compound [I] in which the group R¹ is the protective group for the amino group. Removal of the protective group can be carried out by the conventional manner (for example, acid treatment, base treatment, catalytic reduction, etc.). Among the present reactions, a reaction by the acid treatment can be carried out, for example, at 5° C. to 120° C., a reaction by the base treatment at 5° C. to 40° C., and a reaction by the catalytic reduction at 10° C. to 40° C.
(Method b): The objective Compound [I] in which the group R¹ in the formula [I] is a substituted carbonyl group can be prepared by reacting a corresponding Compound [I] in which the group R¹ is hydrogen atom with the corresponding carboxylic acid compound or its active ester, in the presence or absence of a condensing agent, or reacting Compound [I] with a reactive derivative (acid halide, acid anhydride, active amide, active ester, mixed acid anhydride, etc.) of a carboxylic acid in a solvent in the presence or absence of a base and in the presence or absence of a condensing agent. As the condensing agent, 1,1′-carbonyldiimidazole, 1,3-dicyclohexylcarbodiimide, 1-ethyl-3-(3-dimethylaminopropyl)carbodiimide hydrochloride, isobutyl chloroformate, N-methylmorpholine or benzotriazol-1-yloxytris(dimethylamino)phosphonium hexafluoro phosphorus, etc., can be used. As the active ester of the carboxylic acid compound, an ester with N-hydroxysuccinic imide, N-hydroxyphthalimide, 1-hydroxybenzotriazole or p-nitrophenol can be used. This reaction can be carried out, for example, at −20° C. to 50° C. As the acid halide of the corresponding carboxylic acid compound, an acid chloride, an acid bromide, etc., can be suitably used. Also, as the active amide of the corresponding carboxylic acid compound, an amide with imidazole, etc. can be used.
(Method c): The objective Compound [I] in which the group R¹ in the formula [I] is an optionally substituted heterocyclic group can be prepared by subjecting a corresponding Compound [I] in which the group R¹ is hydrogen atom and a heterocyclic group having a corresponding oxo group to reductive condensation. The reductive condensation can be suitably carried out, for example, according to the method disclosed in (a) Tetrahedron Letters, vol. 31, p. 5595, 1990, (b) Journal of Organic Chemistry, vol. 28, p. 3259, 1963, etc., in the presence of a reducing agent in a suitable solvent. As the reducing agent, any materials which can be suitably used in the reductive amination can be used. Such a reducing agent can be exemplified by a metal reducing agent, for example, metal hydrides [borane hydrides (diborane, etc.), etc.], metal hydride complexes [lithium aluminum hydride, sodium borohydride, etc.], organometal complexes [borane-methyl sulfide, 9-borabicyclononane (9-BBN), triethylsilane, sodium triacetoxyborohydride, sodium cyanoborohydride, etc.] and the like. Also, if necessary, a Lewis acid (titanium tetrachloride, etc.) or an organic acid (acetic acid, etc.) can be used as an additive. Also, in the reductive condensation, it can be also carried out under catalytic hydrogenation conditions in place of existing the reducing agent. For example, it can be carried out by using a suitable catalyst such as platinum catalyst, palladium-carbon, etc., in a suitable solvent under hydrogen stream. Also, it is preferred to add a catalytic amount of an acid in the reductive condensation, and such an acid is exemplified by organic acids such as formic acid, acetic acid, propionic acid, etc., inorganic acids such as hydrochloric acid, nitric acid, sulfuric acid, etc. This reaction can be suitably carried out under cooling to under heating, preferably at 0° C. to 100° C., more preferably at 10° C. to 50° C. The objective Compound [I] in which the group R¹ in the formula [I] is an optionally substituted alkyl group can be prepared by alkylating a corresponding Compound [I] in which the group R¹ in the formula [I] is hydrogen by a conventional manner. This reaction proceeds at 20° C. to 80° C.
(Method d): When the objective Compound [I] in which the group R¹ in the formula [I] is a substituted carbonyl group is a compound having a urea bond, it can be prepared by reacting a corresponding Compound [I] in which the group R¹ is hydrogen atom with a corresponding amine compound by using a urea bond forming agent. As the urea bond forming agent, 1,1′-carbonyldiimidazole, phosgene, etc., are preferred, and, for example, 1,1′-carbonyldiimidazole, carbonyl dihalides such as triphosgene and phosgene can be used. This reaction can be carried out, for example, at 0° C. to 80° C., preferably at 0° C. to 50° C. Also, this reaction can be carried out according to the method disclosed in Japanese Unexamined Patent Publication No. Hei. 10-195037.
(Method e): The objective Compound [I] in which the sulfur atom which is a substituent of the group R¹ in the formula [I] is a group containing a group substituted by oxo group(s) (for example, sulfonyl group, etc.) or the objecting Compound [I] in which the group R¹ is sulfonyl group having a substituent(s) can be prepared by treating a corresponding Compound [I] in which the group R¹ is hydrogen atom with a halogenosulfonyl compound which is a corresponding compound in the presence of a base. As the base, triethylamine, etc., can be used. Moreover, this reaction can be carried out, for example, at 0° C. to 50° C. The objective Compound [I] in which the sulfur atom which is a substituent of the group R¹ in the formula [I] is a group containing a group substituted by two oxo groups (for example, sulfonyl group, etc.) can be prepared by treating a corresponding Compound [I] in which the group R¹ is a group having thio group with an oxidizing agent (for example, 3-chloroperbenzoic acid, peracetic acid, sodium periodate, OXONE, etc.). This reaction suitably proceeds, for example, at −80° C. to 150° C., particularly preferably at 0° C. to 40° C.
(Method f): The objective Compound [I] in which the group R¹ in the formula [I] is an optionally substituted alkyl group can be prepared by alkylating a corresponding Compound [I] in which the group R¹ in the formula [I] is hydrogen by a conventional manner. This reaction can be carried out at 20° C. to 80° C.
(Method g): When the objective Compound [I] in which the group R¹ in the formula [I] has an optionally substituted urethane bond, it can be prepared by reacting a corresponding Compound [I] with a corresponding alcohol compound by using an urethane bond forming agent. As the urethane bond forming agent, for example, 1,1′-carbonyldiimidazole, carbonyl dihalides such as triphosgene and phosgene can be used. This reaction can be carried out, for example, at 0° C. to 80° C., preferably at 0° C. to 50° C. Also, this reaction can be carried out according to the method disclosed in Japanese Unexamined Patent Publication No. Hei. 10-195037.
(Method h): When the objective Compound [I] in which the group R¹ in the formula [I] has an optionally substituted carbonyl group, it can be carried out according to the above method b. The reactive derivative of the carboxylic acid compound, an isocyanate compounds (e.g. trimethylsilyl isocyanate, etc.) can be used. This reaction can be carried out, for example, at 0° C. to 80° C., preferably at 0° C. to 50° C.
(method i): When the objecting Compound [I] in which the group R¹ in the formula [I] has an optionally substituted amido bond, it can be prepared by reacting the corresponding Compound [I] having a free carboxyl group at R¹ with the corresponding amine compound, or by reacting the corresponding Compound [I] having a free amino group at the group R¹ with the corresponding carboxylic acid compound, in the presence or in the absence of a condensing agent. As the condensing agent, there are used 1,1′-carbonyldiimidazole, 1,3-dicyclohexylcarbodiimide, 1-ethyl-3-(3-dimethylaminopropyl)carbodiimide hydrochloride, isobutyl chloroformate or N-methylmorpholine, etc., which are normally used in a reaction to form an amide bond from a carboxylic acid and an amine. The present reaction can proceed, for example, at −20° C. to 50° C.

The solvent to be used in the reactions described in the above-mentioned (Method a) to (Method i) is not specifically limited so long as it does not inhibit the reaction, and, for example, dioxane, ethylene glycol dimethyl ether, dimethylacetamide, dimethylformamide, hexamethylphosphoramide, benzene, tetrahydrofuran, toluene, ethyl acetate, alcohol, dichloromethane, chloroform, carbon tetrachloride, 1,3-dimethyl-2-imidazolidine, acetic acid, diethyl ether, methoxyethane, dimethylsulfoxide, acetonitrile, water or a mixed solvent of the above solvents can be used by optionally selecting them.

Incidentally, of the starting Compound [II] of the present invention, the following Compound [II-a] and Compound [II-b] are novel compounds, and can be prepared, for example, by the following chemical reaction formulae. 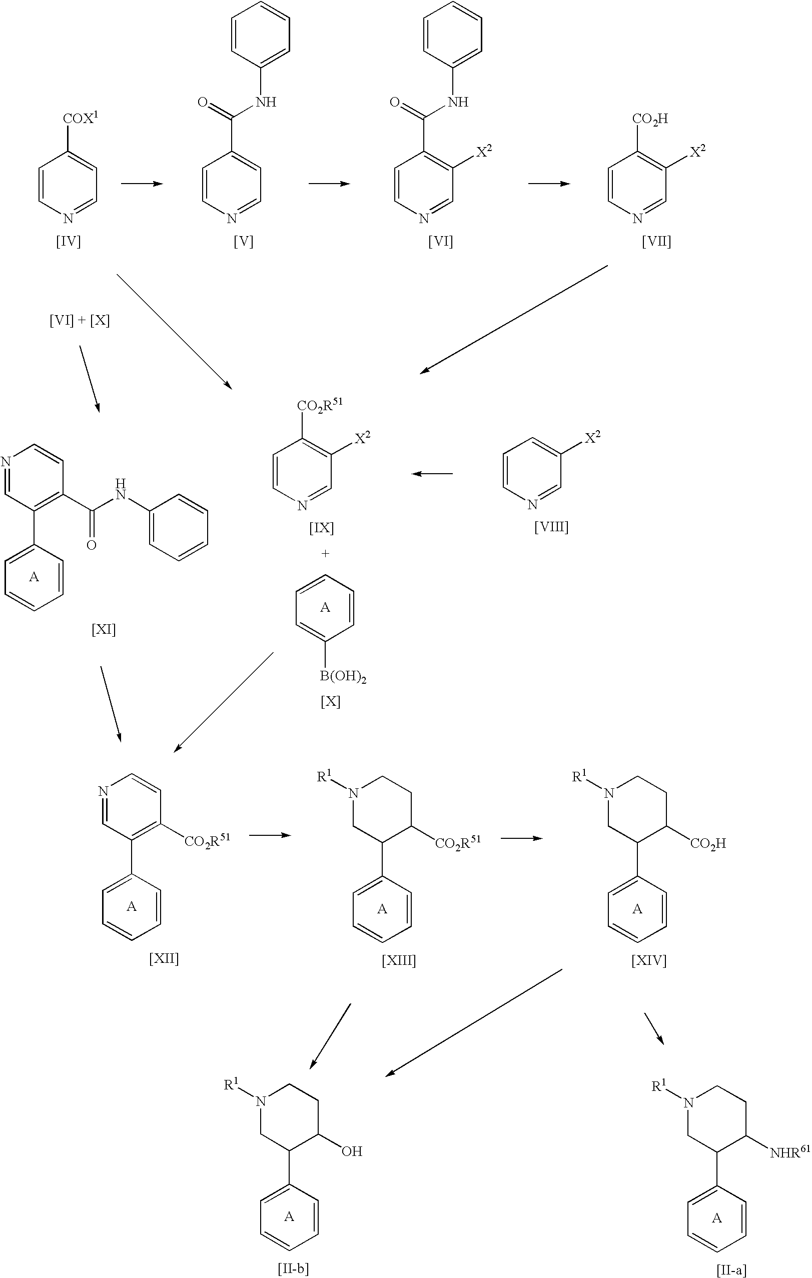
wherein R⁵¹ represents an alkyl group, R⁶¹ represents a protective group for the amino group, X¹ represents a leaving group, X represents a leaving group, and Ring A and R¹ have the same meanings as defined above.

That is, the pyridine compound [IV] is subjected to condensation with aniline to give Compound [V], then, subjecting to halogenation to give Compound [VI], and the aniline is eliminated to give Compound [VII]. Also, Compound [IX] is obtained by esterifying the carboxyl group of Compound [VII], subjecting Compound [VIII] to carbonyl insertion, or esterifying the acyl group of Compound [IV] and then to haloganate. The obtained Compound [IX] and Compound [X] are coupled or Compound [VI] and Compound [X] are coupled to give Compound [XI], and the aniline is eliminated to give Compound [XII], the resulting Compound [XII] is subjected to reduction, then, a substituent of the amino group is introduced to give Compound [XIII]. An ester group of the resulting Compound [XIII] is converted to a carboxyl group to give Compound [XIV]. Moreover, the carboxyl group of the resulting Compound [XIV] is subjected to rearrangement, etc., to give Compound [II-a], or Compound [XIII] or Compound [XIV] is oxidized and then hydrolyzed to give Compound [II-b].

Also, Compound [II-a] can be converted to the following Compound [II-c] according to the following chemical reaction formula. 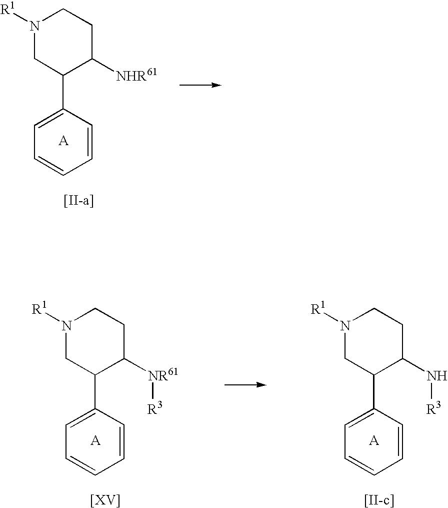
wherein ring A, R¹, R³ and R⁶¹ have the same meanings as defined above.

That is, Compound [XV] is obtained by substituting the amino group of Compound [II-a] with R³, and then, Compound [II-c] is obtained by removing the protective group for the amino group.

Compound [II] (for example, Compound [II-a], Compound [II-b] or Compound [II-c], etc.) has an asymmetric carbon, and optical isomers exist based on the asymmetric carbon. For example, when cis-isomer and trans-isomer are obtained as a mixture, the respective cis-isomer and trans isomer can be obtained separately by a conventional manner such as silica gel chromatography, etc. Also, optical isomers of Compound [II] can be obtained, for example, by optically resolving racemic mixtures of Compound [II], or racemic mixtures of Compound [XIII] where R¹ is hydrogen atom or racemic mixtures of Compound [II] are optically resolved according to a conventional manner to give a corresponding optically active compound, and then, the resulting compound is applied to the above-mentioned reaction.

In the case of a compound wherein R¹ of Compound [XIII] is hydrogen atom, optical resolution can be carried out, for example, by acting Compound [XIII] with N-acyl-optically active amino acid, N-sulfonyl-optically active amino acid or optically active carboxylic acid, and separating and collecting one of the diastereomer salts utilizing the differences in solubility between two kinds of the formed diastereomer salts. The acyl group of the N-acyl-optically active amino acid can be exemplified by, for example, acetyl group, propionyl group, tosyl group or benzyloxycarbonyl group, and the optically active amino acid can be exemplified by, for example, L-phenylalanine, L-leucine, L-glutamine, L-methionine, L-valine, L-threonine, D-phenylalanine or D-phenylglycine. Also, the optically active carboxylic acid is exemplified by mandelic acid, malic acid or tartaric acid derivatives. The tartaric acid derivatives are exemplified by dibenzoyl-L-tartaric acid, di-p-toluoyl-L-tartaric acid, dibenzoyl-D-tartaric acid, di-p-toluoyl-D-tartaric acid, etc.

Also, in the case of Compound [XIV], optical resolution can be carried out by, for example, acting Compound [XIV] with O-alkyl-optically active amino acid or an optically active amine derivative, and separating and collecting one of the diastereomer salts utilizing the differences in solubility between two kinds of the formed diastereomer salts. The optically active amino acid can be exemplified by, for example, L-phenylalanine, L-leucine, L-glutamine, L-methionine, L-valine, L-threonine, D-phenylalanine or D-phenylglycine. The alkyl group of the O-alkyl-optically active amino acid can be exemplified by methyl group, ethyl group, etc. The optically active amine derivative can be exemplified by brucine, quinidine, (S)-α-phenethylamine, (R)-β-phenethylamine, (R)-(−)-1-cyclohexylethylamine, (S)-(+)-1-cyclohexylethylamine, etc.

Further, in preparation of the objective compounds or the starting materials of the present invention, when the starting materials or the intermediates have a functional group, a suitable protecting group can be introduced to each of the functional group by a conventional method, besides the above described method, and if they are not necessary, these protecting groups may be suitably removed.

For example, in the present specification, as the protective group for the amino group, a protective group to be generally used for protecting the amino group for applying the same to a reaction, and it can be specifically exemplified by, for example, an alkoxycarbonyl group such as tert-butoxycarbonyl group, an arylalkoxycarbonyl group such as benzyloxycarbonyl group, etc.

In the present specification, the alkyl group means, for example, a straight or branched alkyl group having 1 to 6 carbon atoms such as methyl group, ethyl group, propyl group, isopropyl group, butyl group, isobutyl group, tert-butyl group, isopentyl group, etc., preferably those having 1 to 4 carbon atoms. The alkenyl group means, for example, a straight or branched alkenyl group having 2 to 7 carbon atoms such as vinyl group, allyl group, propenyl group, isopropenyl group, etc., preferably those having 2 to 4 carbon atoms. The alkoxy group means a straight or branched alkoxy group having 1 to 6 carbon atoms such as methoxy group, ethoxy group, propoxy group, isopropoxy group, butoxy group, etc., preferably those having 1 to 4 carbon atoms. The alkanoyl group means a straight or branched alkanoyl group having 1 to 6 carbon atoms such as formyl group, acetyl group, propionyl group, butyryl group, valeryl group, tert-butylcarbonyl group, etc., preferably those having 1 to 4 carbon atoms. The alkylene group means, for example, a straight or branched alkylene group having 1 to 6 carbon atoms such as methylene group, ethylene group, propylene group, butylene group, pentylene group, hexylene group, etc., preferably those having 1 to 4 carbon atoms. The cycloalkyl group means, for example, a cycloalkyl group having 3 to 8 carbon atoms such as cyclopropyl group, cyclobutyl group, cyclopentyl group, cyclohexyl group, cycloheptyl group, cyclooctyl group, etc., preferably those having 3 to 6 carbon atoms. Further, the halogen atom is exemplified by chlorine atom, bromine atom, fluorine atom and iodine atom. The aryl group is exemplified by an unsaturated hydrocarbon cyclic group such as phenyl group, naphthyl group, phenanthryl group, etc.

### EXAMPLE

### Example 1

To 27 ml of a tetrahydrofuran solution containing 1.42 g of 2-(3,5-bistrifluoromethylphenyl)-2-methylpropionic acid were added a catalytic amount of N,N-dimethylformamide and 1.07 g of thionyl chloride, the resulting mixture was stirred under reflux for 2 hours, and the reaction mixture was concentrated under reduced pressure. To 10 ml of dichloromethane solution containing 1.45 g of (3S,4S)-1-tert-butoxycarbonyl-3-(4-fluoro-2-methylphenyl)-4-(N-methylamino)piperidine cooled to 0° C. were added 0.51 g of triethylamine and 3 ml of dichloromethane solution containing the above-mentioned residue obtained by concentration under reduced pressure, and the resulting mixture was stirred at 0° C. for 1 hour. To the reaction mixture were added dichloromethane and water, and the mixture was separated, the organic layer was washed with brine, dried over anhydrous sodium sulfate, and concentrated under reduced pressure. The obtained residue was purified by silica gel column chromatography (n-hexane:ethyl acetate=19:1→4:1) to give 2.12 g of (3S,4S)-1-tert-butoxycarbonyl-3-(4-fluoro-2-methylphenyl)-4-{N-methyl-2-(3,5-bistrifluoromethylphenyl)isobutyrylamino}piperidine shown in the following Table 1.

### Examples 2 to 5

The corresponding starting materials were used and treated in the same manner as in Example 1, to give compounds shown in the following Table 1 and Table 2.

### Example 6

To 20 ml of a tetrahydrofuran solution containing 0.95 g of 2-(3,5-bistrifluoromethylphenyl)propionic acid were added a catalytic amount of N,N-dimethylformamide and 0.80 g of thionyl chloride, the resulting mixture was stirred under reflux for 3 hours, and the reaction mixture was concentrated under reduced pressure. To 6 ml of a dichloromethane solution containing 0.97 g of (3S,4S)-1-tert-butoxycarbonyl-3-(4-fluoro-2-methylphenyl)-4-(N-methylamino)piperidine cooled to 0° C. were added 0.36 g of triethylamine and 1 ml of a dichloromethane solution containing the above-mentioned residue obtained by concentration under reduced pressure, and the resulting mixture was stirred at 0° C. for 3 hours. To the reaction mixture were added chloroform and water, and the mixture was separated, and the organic layer was washed with brine, dried over anhydrous sodium sulfate, and concentrated under reduced pressure. The obtained residue was purified by silica gel column chromatography (hexane:ethyl acetate=9:1→2:1) to give (a) 0.76 g of (3S,4S)-1-tert-butoxycarbonyl-3-(4-fluoro-2-methylphenyl)-4-{(R)-N-methyl-2-(3,5-bistrifluoromethylphenyl)propionylamino}-piperidine, and (b) 0.82 g of (3S,4S)-1-tert-butoxycarbonyl-3-(4-fluoro-2-methylphenyl)-4-{(S)-N-methyl-2-(3,5-bistrifluoromethylphenyl}propionylaminolpiperidine shown in the following Table 3.

### Example 7

The corresponding starting materials were used and treated in the same manner as in Example 6, to give compounds shown in the following Table 3.

### Example 8

15 ml of an N,N-dimethylformamide solution containing 484 mg of trans-1-tert-butoxycarbonyl-3-(4-fluoro-2-methylphenyl)-4-(N-methylamino)piperidine, 449 mg of 3,5-bistrifluoromethylphenylacetic acid, 253 mg of 1-hydroxybenzotriazole monohydrate, and 316 mg of 1-(3-dimethylaminopropyl)-3-ethylcarbodiimide hydrochloride was stirred at room temperature for 16 hours. To the reaction mixture were added ethyl acetate and semi-saturated brine, and the mixture was separated, and the organic layer was washed successively with semi-saturated brine and an aqueous sodium hydrogen carbonate solution. The organic layer was dried over anhydrous sodium sulfate and concentrated under reduced pressure, and the obtained residue was purified by NH silica gel column chromatography (n-hexane:ethyl acetate=19:1→2:1) to give 380 mg of trans-1-tert-butoxycarbonyl-3-(4-fluoro-2-methylphenyl)-4-{N-methyl-(3,5-bistrifluoromethylphenyl)acetylamino}piperidine shown in the following Table 4.

### Example 9

To 8 ml of an ethyl acetate solution containing 2.12 g of (3S,4S)-l-tert-butoxycarbonyl-3-(4-fluoro-2-methylphenyl)-4-{N-methyl-2-(3,5-bistrifluoromethylphenyl)isobutyrylamino}piperidine was added 26 ml of 4M ethyl acetate solution of hydrochloric acid, and the mixture was stirred at room temperature for 1 hour, and concentrated under reduced pressure. To the residue were added ethyl acetate and water, and the aqueous layer was made basic by using an aqueous sodium carbonate solution and aqueous ammonia and the solutions were separated. The organic layer was dried over anhydrous sodium sulfate, and concentrated under reduced pressure to give 1.39 g of (3S,4S)-3-(4-fluoro-2-methylphenyl)-4-{N-methyl-2-(3,5-bistrifluoromethylphenyl)isobutyrylamino}piperidine shown in the following Table 4.

### Examples 10 to 18

The corresponding starting materials were used and treated in the same manner as in Example 9, to give compounds shown in the following Table 4 to Table 6.

### Example 19

3.3 ml of an N,N-dimethylformamide solution containing 127 mg of (3S,4S)-3-(4-fluoro-2-methylphenyl)-4-{N-methyl-2-(3,5-bistrifluoromethylphenyl)isobutyrylamino}piperidine, 37 mg of β-hydroxyisovaleric acid, 48 mg of 1-hydroxybenzotriazole monohydrate and 60 mg of 1-(3-dimethylaminopropyl)-3-ethylcarbodiimide hydrochloride was stirred at 40° C. for 16 hours. To the reaction mixture were added ethyl acetate and semi-saturated brine, and the mixture was separated, and then, the organic layer was washed successively with semi-saturated brine and a saturated aqueous sodium hydrogen carbonate solution. The organic layer was dried by using CHEM ELUT (trade name, available from VARIAN INC.), and concentrated under reduced pressure. The obtained residue was purified by NH thin-layer silica gel column chromatography (chloroform:ethyl acetate=20:1) to give 148 mg of (3S,4S)-3-(4-fluoro-2-methylphenyl)-1-(3-hydroxy-3-methylbutyryl)-4-{N-methyl-2-(3,5-bistrifluoromethylphenyl)isobutyrylamino}piperidine shown in the following Table 7.

### Examples 20 to 74

The corresponding starting materials were used and treated in the same manner as in Example 19, to give compounds shown in the following Table 7 to Table 17.

### Example 75

To 1.5 ml of an N,N-dimethylformamide solution containing 30 mg of the compound obtained in Example 26 cooled to 0° C. was added 2 mg of sodium hydride, the mixture was stirred at 0° C. for 30 minutes, 8 mg of methyl iodide was then added to the mixture and the resulting mixture was stirred at room temperature for 2 hours. To the reaction mixture were added ethyl acetate and semi-saturated brine, the mixture was separated, and the organic layer was washed with semi-saturated brine. The organic layer was dried by using CHEM ELUT (trade name, available from VARIAN INC.), and concentrated under reduced pressure. The obtained residue was purified by basic thin layer silica gel chromatography (chloroform:n-hexane: ethyl acetate=1:1:1) to give 11 mg of (3S,4S)-1-(N-acetyl-N-methyl-amino)acetyl-3-(4-fluoro-2-methylphenyl)-4-{N-methyl-2-(3,5-bistrifluoromethylphenyl)isobutyrylamino}piperidine shown in the following Table 18.

### Examples 76 to 86

The corresponding starting materials were used and treated in the same manner as in Example 75, to give compounds shown in the following Table 18 and Table 19.

### Example 87

To 3.3 ml of a dichloromethane solution containing 127 mg of (3S,4S)-3-(4-fluoro-2-methylphenyl)-4-{N-methyl-2-(3,5-bistrifluoromethylphenyl)isobutyrylamino}piperidine and 42 mg of 1-acetyl-4-piperidone was added 1 drop of acetic acid, the mixture was stirred at room temperature for 1 hour. Then, 106 mg of sodium triacetoxyborohydride was added to the mixture, and the resulting mixture was stirred at 40° C. for 16 hours. To the reaction mixture were added chloroform and a saturated sodium hydrogen carbonate solution and the mixture was separated. The organic layer was dried by using CHEM ELUT (trade name, available from VARIAN INC.), and concentrated under reduced pressure. The obtained residue was purified by NH thin-layer silica gel column chromatography (chloroform:ethyl acetate=20:1) to give 86 mg of (3S,4S)-1-(1-acetyl-piperidin-4-yl)-3-(4-fluoro-2-methylphenyl)-4-{N-methyl-2-(3,5-bistrifluoromethylphenyl)isobutyrylamino}piperidine shown in the following Table 20.

### Examples 88 to 92

The corresponding starting materials were used and treated in the same manner as in Example 87, to give compounds shown in the following Table 20.

### Example 93

(1) To 15 ml of a dichloromethane solution containing 0.5 g of (3S,4S)-3-(4-fluoro-2-methylphenyl)-4-{N-methyl-2-(3,5-bistrifluoromethylphenyl)isobutyrylamino}piperidine and 0.25 g of 1-benzyloxycarbonylazetidin-3-one was added 30 mg of acetic acid, and the mixture was stirred at room temperature for 0.5 hour. Then, 0.42 g of sodium triacetoxyborohydride was added to the mixture, and the resulting mixture was stirred at room temperature for 16 hours. To the reaction mixture were added chloroform and a saturated sodium hydrogen carbonate solution and the mixture was separated. The organic layer was dried by using CHEM ELUT (trade name, available from VARIAN INC.), and concentrated under reduced pressure. The obtained residue was purified by basic silica gel column chromatography (hexane:ethyl acetate=19:1→2:1) to give 0.54 g of (3S,4S)-1-(1-benzyloxycarbonylazetidin-3-yl)-3-(4-fluoro-2-methylphenyl)-4-{N-methyl-2-(3,5-bistrifluoromethylphenyl)isobutyrylamino}piperidine shown in the following Table 20.
(2) To 25 ml of a methanol solution containing 0.54 g of the compound obtained in the above-mentioned (1) was added 0.14 g of 10% palladium carbon, and the mixture was stirred under hydrogen atmosphere at room temperature for 16 hours. The reaction mixture was filtered through membrane filter, and the filtrate was concentrated under reduced pressure to give 0.41 g of (3S,4S)-1-(azetidin-3-yl)-3-(4-fluoro-2-methylphenyl)-4-{N-methyl-2-(3,5-bistrifluoromethylphenyl)isobutyrylamino}piperidine shown in the following Table 20.

### Examples 94 to 96

The corresponding starting materials were used and treated in the same manner as in Example 87, to give compounds shown in the following Table 21.

### Example 97

The corresponding starting materials were used and treated in the same manner as in Example 93, to give compound shown in the following Table 21.

### Examples 98 to 100

The corresponding starting materials were used and treated in the same manner as in Example 87, to give compounds shown in the following Table 22 and Table 23.

### Example 101

To 1.5 ml of an N,N-dimethylformamide solution containing 35 mg of (3S,4S)-3-(4-fluoro-2-methylphenyl)-4-{N-methyl-2-(3,5-bistrifluoromethylphenyl)isobutyrylaminolpiperidine were added 14 mg of triethylamine and 15 mg of 2-bromoethylmethyl ether, and the mixture was stirred at room temperature for 16 hours. To the reaction mixture were added ethyl acetate and semi-saturated brine, the mixture was separated, and the organic layer was washed with semi-saturated brine. The organic layer was dried by using CHEM ELUT (trade name, available from VARIAN INC.), and concentrated under reduced pressure. The obtained residue was purified by thin layer silica gel chromatography (chloroform:methanol=19:1) to give 22 mg of (3S,4S)-3-(4-fluoro-2-methylphenyl)-1-(2-methoxyethyl)-4-1N-methyl-2-(3,5-bistrifluoromethylphenyl)isobutyrylamino}-piperidine shown in the following Table 24.

### Example 102

The corresponding starting materials were used and treated in the same manner as in Example 101, to give a compound shown in the following Table 24.

### Example 103

1.5 ml of an acetonitrile solution containing 35 mg of (3S,4S)-3-(4-fluoro-2-methylphenyl)-4-{N-methyl-2-(3,5-bistrifluoromethylphenyl)isobutyrylamino}piperidine, 13 mg of 2-chloro-N,N′-dimethylacetamide, and 21 mg of potassium carbonate was stirred under reflux for 16 hours. To the reaction mixture were added ethyl acetate and water, and the mixture was separated. The obtained organic layer was washed successively with a saturated aqueous sodium hydrogen carbonate solution and saturated brine. The organic layer was dried by using CHEM ELUT (trade name, available from VARIAN INC.), and concentrated under reduced pressure. The obtained residue was purified by basic thin layer silica gel chromatography (hexane:ethyl acetate=1:1) to give 31 mg of (3S,4S)-1-(1,1-dimethylcarbamoylmethyl)-3-(4-fluoro-2-methylphenyl)-4-{N-methyl-2-(3,5-bistrifluoromethylphenyl)isobutyrylamino}piperidine shown in the following Table 24.

### Examples 104 and 105

The corresponding starting materials were used and treated in the same manner as in Example 103, to give compounds shown in the following Table 24.

### Example 106

To 1.5 ml of an ethanol solution containing 35 mg of (3S,4S)-3-(4-fluoro-2-methylphenyl)-4-{N-methyl-2-(3,5-bistrifluoromethylphenyl)isobutyrylamino}piperidine was added 10 mg of N,N-dimethyl acrylamide, and the mixture was stirred under reflux for 16 hours. To the reaction mixture were added ethyl acetate and semi-saturated brine, and the mixture was separated. The organic layer was dried by using CHEM ELUT (trade name, available from VARIAN INC.), and concentrated under reduced pressure. The obtained residue was purified by NH thin layer silica gel chromatography (hexane:ethyl acetate=1:2) to give 30 mg of (3S,4S)-1-(2,2-dimethylcarbamoylethyl)-3-(4-fluoro-2-methylphenyl)-4-{N-methyl-2-(3,5-bistrifluoromethylphenyl)isobutyrylamino}piperidine shown in the following Table 24.

### Example 107

To 1.5 ml of a dichloromethane solution containing 20 mg of N-(2-hydroxyethyl)succinimide was added 21 mg of triethylamine, and the mixture was cooled to 0° C. To the mixture was added 24 mg of methanesulfonyl chloride, and the resulting mixture was stirred at room temperature for 16 hours, and then, the reaction mixture was concentrated under reduced pressure. A mixture comprising 35 mg of (3S,4S)-3-(4-fluoro-2-methylphenyl)-4-{N-methyl-2-(3,5-bistrifluoromethylphenyl)isobutyrylamino}piperidine, 31 mg of potassium carbonate, and 2 ml of an acetonitrile solution containing the above-mentioned residue concentrated under reduced pressure was stirred under reflux for 16 hours. To the reaction mixture were added ethyl acetate and water, the mixture was separated, and the obtained organic layer was washed successively with a saturated aqueous sodium hydrogen carbonate solution and saturated brine. The organic layer was dried by using CHEM ELUT (trade name, available from VARIAN INC.), and concentrated under reduced pressure. The obtained residue was purified by NH thin layer silica gel chromatography (hexane:ethyl acetate=1:1) to give 16 mg of (3S,4S)-1-{2-(2,5-dioxopyrrolidin-1-yl)ethyl}-3-(4-fluoro-2-methylphenyl)-4-{N-methyl-2-(3,5-bistrifluoromethylphenyl)isobutyrylamino}piperidine shown in the following Table 24.

### Examples 108 to 110

The corresponding starting materials were used and treated in the same manner as in Example 103, to give compounds shown in the following Table 25.

### Example 111

The corresponding starting materials were used and treated in the same manner as in Example 106, to give a compound shown in the following Table 25.

### Example 112

To 2 ml of a dichloromethane solution containing 35 mg of (3S,4S)-1-(azetidin-3-yl)-3-(4-fluoro-2-methylphenyl)-4-{N-methyl-2-(3,5-bistrifluoromethylphenyl)isobutyrylamino}piperidine cooled to 0° C. were added 8 mg of triethylamine and 6 mg of acetyl chloride, and the mixture was stirred at 0° C. for 2 hours. To the reaction mixture were added chloroform and water, the mixture was separated, and the organic layer was washed with saturated brine. The organic layer was dried by using CHEM ELUT (trade name, available from VARIAN INC.), and concentrated under reduced pressure. The obtained residue was purified by thin layer silica gel chromatography (chloroform:methanol=19:1) to give 33 mg of (3S,4S)-1-(1-acetylazetidin-3-yl)-3-(4-fluoro-2-methylphenyl)-4-{N-methyl-2-(3,5-bistrifluoromethylphenyl)isobutyrylamino}piperidine shown in the following Table 26.

### Examples 113 to 117

The corresponding starting materials were used and treated in the same manner as in Example 112, to give compounds shown in the following Table 26.

### Example 118

The corresponding starting materials were used and treated in the same manner as in Example 19, to give a compound shown in the following Table 26.

### Examples 119 to 124

The corresponding starting materials were used and treated in the same manner as in Example 112, to give compounds shown in the following Table 27.

### Example 125

The corresponding starting materials were used and treated in the same manner as in Example 19, to give a compound shown in the following Table 27.

### Example 126

To 1 ml of a dichloromethane solution containing 40 mg of the compound obtained in Example 91 cooled to 0° C. was added 23 mg of meta-chloroperbenzoic acid, and the mixture was stirred at room temperature for 16 hours. To the reaction mixture was added an aqueous sodium hydrogen sulfite solution, and the resulting mixture was stirred at room temperature for 30 minutes, chloroform and water were added to the mixture, and the mixture was separated. The organic layer was dried by using CHEM ELUT (trade name, available from VARIAN INC.), and concentrated under reduced pressure. The obtained residue was purified by thin layer silica gel chromatography (chloroform:methanol=19:1) to give 24 mg of (3S,4S)-3-(4-fluoro-2-methylphenyl)-1-(tetrahydrothiopyran-1-oxid-4-yl)-4-{N-methyl-2-(3,5-bistrifluoromethylphenyl)isobutyrylamino}piperidine shown in the following Table 28.

### Example 127

To 1.5 ml of a dichloromethane solution containing 24 mg of the compound obtained in Example 91 was added 18 mg of methanesulfonic acid, and the mixture was stirred at room temperature for 10 minutes, and then, cooled to 0° C. To the mixture was added 14 mg of meta-chloroperbenzoic acid, and the mixture was stirred at room temperature for 3 hours. To the reaction mixture were added an aqueous sodium hydrogen sulfite solution and 1M aqueous sodium hydroxide solution, the resulting mixture was stirred at room temperature for 30 minutes, chloroform and water were added to the mixture, and the mixture was separated. The organic layer was dried by using CHEM ELUT (trade name, available from VARIAN INC.), and concentrated under reduced pressure. The obtained residue was purified by thin layer silica gel chromatography (hexane:ethyl acetate=1:1) to give 20 mg of (3S,4S)-3-(4-fluoro-2-methylphenyl)-1-(tetrahydrothiopyran-1,1-dioxid-4-yl)-4-{N-methyl-2-(3,5-bistrifluoromethylphenyl)isobutyrylamino}piperidine shown in the following Table 28.

### Example 128

The corresponding starting materials were used and treated in the same manner as in Example 127, to give a compound shown in the following Table 28.

### Example 129

The corresponding starting materials were used and treated in the same manner as in Example 126, to give a compound shown in the following Table 29.

### Examples 130 and 131

The corresponding starting materials were used and treated in the same manner as in Example 127, to give compounds shown in the following Table 29.

### Example 132

To 1 ml of a dichloromethane solution containing 127 mg of (3S,4S)-3-(4-fluoro-2-methylphenyl)-4-{N-methyl-2-(3,5-bistrifluoromethylphenyl)isobutyrylamino}piperidine cooled to 0° C. were added 11 mg of 4-morpholinecarbonyl chloride and 7.4 mg of triethylamine, and the mixture was stirred at 0° C. for 2 hours. To the reaction mixture were added dichloromethane and water, and the mixture was separated. The organic layer was dried by using CHEM ELUT (trade name, available from VARIAN INC.), and concentrated under reduced pressure. The obtained residue was purified by basic thin-layer silica gel column chromatography (chloroform:ethyl acetate=20:1) to give 39 mg of (3S,4S)-3-(4-fluoro-2-methylphenyl)-4-{N-methyl-2-(3,5-bistrifluoromethylphenyl)isobutyrylamino}-1-(4-morpholinecarbonyl)piperidine shown in the following Table 30.

### Example 133

The corresponding starting materials were used and treated in the same manner as in Example 132, to give a compound shown in the following Table 30.

### Example 134

1.5 ml of a tetrahydrofuran solution containing 50 mg of (3S,4S)-3-(4-fluoro-2-methylphenyl)-4-{N-methyl-2-(3,5-bistrifluoromethylphenyl)isobutyrylamino}piperidine and 16 mg of 1,1′-carbonyldiimidazole was stirred at 50° C. for 1 hour. To the reaction mixture were added ethyl acetate and water, the mixture was separated, and the organic layer was washed with water. The organic layer was dried by using CHEM ELUT (trade name, available from VARIAN INC.), and concentrated under reduced pressure. To the obtained residue were added 2 ml of acetonitrile and 0.85 g of methyl iodide, the mixture was stirred at 70° C. for 1 hour, and concentrated under reduced pressure. To the obtained residue were added 1.5 ml of tetrahydrofuran, 14 mg of 1-acetylpiperazine and 0.61 g of triethylamine, and the mixture was stirred at room temperature for 16 hours. To the reaction mixture were added ethyl acetate and water, and the mixture was separated. The organic layer was dried by using CHEM ELUT (trade name, available from VARIAN INC.), and concentrated under reduced pressure. The obtained residue was purified by basic thin-layer silica gel column chromatography (hexane:ethyl acetate=2:1) to give 39 mg of (3S,4S)-1-(1-acetylpiperazinecarbonyl)-3-(4-fluoro-2-methylphenyl)-4-{N-methyl-2-(3,5-bistrifluoromethylphenyl)isobutyrylamino}piperidine shown in the following Table 30.

### Examples 135 to 140

The corresponding starting materials were used and treated in the same manner as in Example 134, to give compounds shown in the following Table 30 and Table 31.

### Example 141

The corresponding starting materials were used and treated in the same manner as in Example 126, to give a compound shown in the following Table 31.

### Examples 142 and 143

The corresponding starting materials were used and treated in the same manner as in Example 134, to give compounds shown in the following Table 31 and Table 32.

### Examples 144 to 147

The corresponding starting materials were used and treated in the same manner as in Example 19, to give compounds shown in the following Table 33.

### Examples 148 and 149

The corresponding starting materials were used and treated in the same manner as in Example 87, to give compounds shown in the following Table 33.

### Examples 150 to 153

The corresponding starting materials were used and treated in the same manner as in Example 19, to give compounds shown in the following Table 34.

### Examples 154 and 155

The corresponding starting materials were used and treated in the same manner as in Example 87, to give compounds shown in the following Table 34.

### Examples 156 to 173

The corresponding starting materials were used and treated in the same manner as in Example 1, to give compounds shown in the following Table 35 to 39.

### Examples 174 to 191

The corresponding starting materials were used and treated in the same manner as in Example 9, to give compounds shown in the following Table 40 to 44.

### Example 192

The corresponding starting materials were used and treated in the same manner as in Example 19, to give compound shown in the following Table 45.

### Examples 193 and 194

The corresponding starting materials were used and treated in the same manner as in Example 87, to give compounds shown in the following Table 45.

### Example 195

The corresponding starting materials were used and treated in the same manner as in Example 127, to give compound shown in the following Table 45.

### Example 196

The corresponding starting materials were used and treated in the same manner as in Example 19, to give compound shown in the following Table 46.

### Examples 197 and 198

The corresponding starting materials were used and treated in the same manner as in Example 87, to give compounds shown in the following Table 46.

### Example 199

The corresponding starting materials were used and treated in the same manner as in Example 127, to give compound shown in the following Table 46.

### Examples 200 to 202

The corresponding starting materials were used and treated in the same manner as in Example 19, to give compounds shown in the following Table 47.

### Examples 203 to 208

The corresponding starting materials were used and treated in the same manner as in Example 87, to give compounds shown in the following Table 47 and 48.

### Examples 209 to 211

The corresponding starting materials were used and treated in the same manner as in Example 127, to give compounds shown in the following Table 48.

### Examples 212 to 214

The corresponding starting materials were used and treated in the same manner as in Example 19, to give compounds shown in the following Table 49.

### Examples 215 to 220

The corresponding starting materials were used and treated in the same manner as in Example 87, to give compounds shown in the following Table 49 and 50.

### Examples 221 to 223

The corresponding starting materials were used and treated in the same manner as in Example 127, to give compounds shown in the following Table 50.

### Examples 224 and 225

The corresponding starting materials were used and treated in the same manner as in Example 19, to give compounds shown in the following Table 51.

### Examples 226 to 229

The corresponding starting materials were used and treated in the same manner as in Example 87, to give compounds shown in the following Table 51.

### Examples 230 and 231

The corresponding starting materials were used and treated in the same manner as in Example 127, to give compounds shown in the following Table 51.

### Examples 232 and 233

The corresponding starting materials were used and treated in the same manner as in Example 19, to give compounds shown in the following Table 52.

### Examples 234 to 237

The corresponding starting materials were used and treated in the same manner as in Example 87, to give compounds shown in the following Table 52.

### Examples 238 and 239

The corresponding starting materials were used and treated in the same manner as in Example 127, to give compounds shown in the following Table 52.

### Example 240

The corresponding starting materials were used and treated in the same manner as in Example 19, to give compound shown in the following Table 53.

### Examples 241 and 242

The corresponding starting materials were used and treated in the same manner as in Example 87, to give compounds shown in the following Table 53.

### Example 243

The corresponding starting materials were used and treated in the same manner as in Example 127, to give compound shown in the following Table 53.

### Examples 244 to 246

The corresponding starting materials were used and treated in the same manner as in Example 19, to give compounds shown in the following Tables 54 and 55.

### Examples 247 to 252

The corresponding starting materials were used and treated in the same manner as in Example 87, to give compounds shown in the following Tables 55 to 58.

### Examples 253 to 255

The corresponding starting materials were used and treated in the same manner as in Example 127, to give compounds shown in the following Tables 58 and 59.

### Example 256

The corresponding starting materials were used and treated in the same manner as in Example 19, to give compound shown in the following Table 60.

### Examples 257 and 258

The corresponding starting materials were used and treated in the same manner as in Example 87, to give compounds shown in the following Table 60.

### Example 259

The corresponding starting materials were used and treated in the same manner as in Example 127, to give compound shown in the following Table 60.

### Example 260

The corresponding starting materials were used and treated in the same manner as in Example 19, to give compound shown in the following Table 61.

### Example 261

The corresponding starting materials were used and treated in the same manner as in Example 87, to give compound shown in the following Table 61.

### Examples 262 to 279

The corresponding starting materials were used and treated in the same manner as in Example 19 to give compound shown in the following Tables 62 and 63.

### Examples 280 and 281

The corresponding starting materials were used and treated in the same manner as in Example 75 to give compound shown in the following Table 64.

### Examples 282 to 284

The corresponding starting materials were used and treated in the same manner as in Example 19 to give compound shown in the following Table 64.

### Example 285

To 2 ml of a dichloromethane solution containing 78 mg of (3S,4S)-1-(azetidin-3-yl)-3-(4-fluoro-2-methylphenyl)-4-{N-methyl-2-(3,5-bistrifluoromethylphenyl)isobutylamino}piperidine was added 21 mg of triethylamine and 29 mg of ethyloxalyl chloride, and the mixture was stirred at room temperature for 16 hours. To the reaction mixture were added chloroform and water, the mixture was separated, and the organic layer was washed with brine. The organic layer was dried by using CHEM ELUT (trade name, available from VARIAN INC.), and concentrated under reduced pressure. To the obtained residue was added 1 ml of ethanol and 1 ml of 28% aqueous ammonium solution, and the mixture was stirred at 100° C. for 4.5 hours. To the reaction mixture were added ethyl acetate and water, and the mixture was separated, and the organic layer was washed with brine. The organic layer was dried by using CHEM ELUT (trade name, available from VARIAN INC.), and concentrated under reduced pressure. The obtained residue was purified by thin-layer silica gel column chromatography (chloroform:methanol=19:1) to give 50 mg of (3S,4S)-1-(1-aminooxalylazetidin-3-yl)-3-(4-fluoro-2-methylphenyl)-4-{N-methyl-2-(3,5-bistrifluoromethylphenyl)isobutyrylamino}piperidine shown in the following Table 64.

### Examples 286 and 287

The corresponding starting materials were used and treated in the same manner as in Example 19, to give compounds shown in the following Table 65.

### Example 288

To 1 ml of a dichloromethane solution containing 45 mg of (3S,4S)-1-(azetidin-3-yl)-3-(4-fluoro-2-methylphenyl)-4-{N-methyl-2-(3,5-bistrifluoromethylphenyl)isobutylamino}piperidine was added 0.041 ml of triethylamine and the mixture was cooling down 0° C. The mixture was added 9.5 mg of triphosgene and stirred at room temperature for 2 hours. To the reaction mixture were added ethyl acetate and semi-saturated brine, and the mixture was separated, and the organic layer was washed with brine. The organic layer was dried by using CHEM ELUT (trade name, available from VARIAN INC.), and concentrated under reduced pressure. To the obtained residue was added 0.5 mg of dimethylaminopyridine, 1 ml of tetrahydrofran, 0.15 ml of ethylene glycol and 0.11 ml of triethylamine, and the mixture was stirred at room temperature for 16 hours. To the reaction mixture were added ethyl acetate and water, and the mixture was separated, and the organic layer was washed with brine. The organic layer was dried by using CHEM ELUT (trade name, available from VARIAN INC.), and concentrated under reduced pressure. The obtained residue was purified by thin-layer silica gel column chromatography (chloroform:methanol=9:1) to give 38 mg of (3S,4S)-1-{1-(2-hydroxyethylaminocarbonyl)azetidin-3-yl}-3-(4-fluoro-2-methylphenyl)-4-{N-methyl-2-(3,5-bistrifluoromethylphenyl)isobutyrylamino}piperidine shown in the following Table 65.

### Example 289

To 2 ml of a dichloromethane solution containing 45 mg of (3S,4S)-1-(azetidin-3-yl)-3-(4-fluoro-2-methylphenyl)-4-{N-methyl-2-(3,5-bistrifluoromethylphenyl)isobutylamino}piperidine was added 41 mg of trimethylsilylisocianate, and the mixture was stirred at room temperature for 16 hours. To the reaction mixture were added chloroform and a saturated aqueous sodium carbonate solution. The mixture was separated, and the organic layer was washed with brine. The organic layer was dried by using CHEM ELUT (trade name, available from VARIAN INC.), and concentrated under reduced pressure. The obtained residue was purified by thin-layer silica gel column chromatography (chloroform:methanol=9:1) to give 40 mg of (3S,4S)-1-(1-aminocarbonylazetidin-3-yl)-3-(4-fluoro-2-methylphenyl)-4-{N-methyl-2-(3,5-bistrifluoromethylphenyl)isobutyrylamino}piperidine shown in the following Table 65.

### Example 290

The corresponding starting materials were used and treated in the same manner as in Example 134, to give compounds shown in the following Table 65.

### Examples 291 and 292

The corresponding starting materials were used and treated in the same manner as in Example 106, to give compounds shown in the following Table 65.

### Reference Example 1

(1) To a solution comprising 1.5 ml of methanol and 3.6 ml of dichloromethane containing 3.3 g of 3,5-bistrifluoromethylphenylacetic acid was added 36 μl of conc. sulfuric acid, and the mixture was stirred under reflux for 16 hours. To the reaction mixture were added dichloromethane and water, the mixture was separated, and the organic layer was washed with an aqueous sodium hydrogen carbonate solution, dried over anhydrous sodium sulfate, and concentrated under reduced pressure. To the obtained residue was added 100 ml of tetrahydrofuran, the mixture was cooled to −78° C., and 3.37 g of potassium tert-butoxide was added to the mixture, and the resulting mixture was stirred at the same temperature for 30 minutes. Moreover, 5.09 g of methyl iodide was added to the mixture, and the resulting mixture was stirred at room temperature for 16 hours. To the reaction mixture were added ethyl acetate and water, the mixture was separated, and the organic layer was dried over anhydrous magnesium sulfate, and concentrated under reduced pressure. The obtained residue was purified by silica gel column chromatography (n-hexane:ethyl acetate=100:0→19:1) to give 2.9 g of methyl 2-(3,5-bistrifluoromethylphenyl)-2-methylpropionate shown in the following Table 66.
(2) To 40 ml of a methanol solution containing 2.86 g of the compound obtained by the above-mentioned (1) was added 42 ml of 2M aqueous sodium hydroxide solution, and the mixture was stirred at 80° C. for 16 hours. The reaction mixture was cooled to room temperature, 14 ml of 6M aqueous hydrochloric acid solution, ethyl acetate and water were added to the mixture, the mixture was separated, and the organic layer was washed with brine, dried over anhydrous magnesium sulfate, and concentrated under reduced pressure. The obtained residue was recrystallized from dichloromethane and hexane to give 2.50 g of 2-(3,5-bistrifluoromethylphenyl)-2-methylpropionic acid shown in the following Table 66.

### Reference Example 2

(1) To a solution comprising 2.2 ml of methanol and 5.5 ml of dichloromethane containing 4.8 g of 3,5-bistrifluoromethylphenylacetic acid was added 54 μl of conc. sulfuric acid, and the mixture was stirred under reflux for 16 hours. To the reaction mixture were added dichloromethane and water, the mixture was separated, and the obtained organic layer was washed with a saturated aqueous sodium hydrogen carbonate solution, dried over anhydrous magnesium sulfate, and concentrated under reduced pressure. To the obtained residue was added 300 ml of tetrahydrofuran, the mixture was cooled to −20° C., 0.70 g of sodium hydride was added to the mixture and the resulting mixture was stirred at the same temperature for 0.5 hour. Moreover, 2.5 g of methyl iodide was added to the mixture, and the resulting mixture was stirred at room temperature for 16 hours. To the reaction mixture were added ethyl acetate and semi-saturated brine, the mixture was separated, and the obtained organic layer was washed with semi-saturated brine, dried over anhydrous magnesium sulfate, and concentrated under reduced pressure. The obtained residue was purified by silica gel column chromatography (hexane:ethyl acetate=20:1→15:1) to give 2.4 g of methyl 2-(3,5-bistrifluoromethylphenyl)propionate shown in the following Table 66.
(2) To 90 ml of a methanol solution containing 6.7 g of methyl 2-(3,5-bistrifluoromethylphenyl)propionate was added 100 ml of 2M aqueous sodium hydroxide solution, and the mixture was stirred at 80° C. for 6 hours. The reaction mixture was cooled to room temperature, 33 ml of 6M aqueous hydrochloric acid solution, ethyl acetate and water were added to the mixture, the mixture was separated, and the organic layer was washed with saturated brine, dried over anhydrous magnesium sulfate, and concentrated under reduced pressure. The obtained residue was recrystallized from dichloromethane and hexane to give 4.1 g of 2-(3,5-bistrifluoromethylphenyl)propionic acid shown in the following Table 66.

### Reference Example 3

(1) 320 ml of a tetrahydrofuran solution containing 22.4 ml of diisopropylamine was cooled to −70° C. or lower with a dry ice-acetone bath, 100 ml of n-butyl lithium (1.6M hexane solution) was added dropwise, and the mixture was stirred at the same temperature for 30 minutes. To the solution was added dropwise 250 ml of a tetrahydrofuran solution containing 25 g of 3-bromopyridine over 4 hours, and the mixture was further stirred at −70° C. or lower for 1 hour. To the solution was added 8.8 g of dry ice which had been finely pulverized after wiping the surface well, the resulting mixture was stirred for 1 hour, and the temperature of the mixture was gradually raised to room temperature. The solvent and the excess carbon dioxide were completely removed under reduced pressure, the residue was dissolved in 300 ml of N,N-dimethylformamide, 27.6 g of potassium carbonate and 12.6 ml of methyl iodide were added to the solution, and the mixture was stirred at room temperature for 16 hours. Ethyl acetate and an aqueous sodium bicarbonate solution were added to the mixture, the mixture was separated, and the organic layer was washed with water and brine. The organic layer was dried over anhydrous magnesium sulfate, and concentrated under reduced pressure. The obtained residue was purified by silica gel column chromatography (n-hexane:ethyl acetate=4:1) to give 13.5 g of methyl 3-bromoisonicotinate shown in the following Table 66.
(2) To 120 ml of an N,N-dimethylformamide solution containing 12 g of the compound obtained in the above-mentioned (1) were added 9.3 g of 4-fluoro-2-methylphenylboric acid, 19.6 g of cesium carbonate, 1.12 g of palladium acetate and 2.63 g of triphenylphosphine, and the mixture was stirred at 70° C. for 1 hour. After completion of the reaction, ethyl acetate and brine were added to the mixture, and insoluble materials were filtered off. The filtrate was washed successively with brine and water, dried over anhydrous magnesium sulfate, and concentrated under reduced pressure. The obtained residue was purified by silica gel column chromatography (n-hexane:ethyl acetate=4:1) to give 7.9 g of methyl 3-(4-fluoro-2-methylphenyl)isonicotinate shown in the following Table 66.
(3) To 100 ml of a methanol solution containing 2.5 g of the compound obtained in the above-mentioned (2) were added 600 mg of platinum oxide and 8 ml of conc. hydrochloric acid to carry out hydrogen substitution 5 times repeatedly. Then, under hydrogen pressure of 101 kPa, the mixture was stirred at room temperature for 24 hours. To the solution was added 100 ml of water, and the mixture was filtered through Celite, and the filtrate was concentrated under reduced pressure. The remaining aqueous solution was neutralized by sodium carbonate, and after adding aqueous ammonia, the mixture was extracted twice with chloroform. The combined organic layers were dried over anhydrous sodium sulfate, and concentrated under reduced pressure. To 25 ml of a dichloromethane solution containing the residue was added 5 g of di-tert-butyl-dicarbonate, and the mixture was stirred at room temperature for 1 hour, and then, concentrated under reduced pressure. The obtained residue was purified by silica gel column chromatography (n-hexane: ethyl acetate=85:15) to give 1.3 g of cis-1-tert-butoxycarbonyl-3-(4-fluoro-2-methylphenyl)-4-methoxycarbonylpiperidine shown in the following Table 67.
(4) To 500 ml of a methanol solution containing 54.9 g of cis-1-tert-butoxycarbonyl-3-(4-fluoro-2-methylphenyl)-4-methoxycarbonylpiperidine obtained in the same manner as in the above-mentioned (3) was added 59.8 ml of sodium methylate (28% methanol solution), and the mixture was stirred under reflux for 3 hours. After cooling to room temperature, 390 ml of 2M aqueous sodium hydroxide solution and 200 ml of tetrahydrofuran were added to the mixture, and the resulting mixture was further stirred for 2 hours. To the reaction mixture was added 2M aqueous hydrochloric acid solution to neutralize the mixture, and the solvent was removed under reduced pressure. To the obtained residue was added chloroform, and the mixture was separated. The organic layer was washed with saturated brine, dried over anhydrous magnesium sulfate, and concentrated under reduced pressure. The obtained residue was recrystallized from ethyl acetate-n-hexane to give 42.3 g of trans-1-tert-butoxycarbonyl-4-carboxyl-3-(4-fluoro-2-methylphenyl)piperidine shown in the following Table 67.
(5) To 1000 ml of an ethyl acetate solution containing 40.5 g of the compound obtained in the above-mentioned (4) was added 100 ml of an ethyl acetate solution containing 7.27 g of (S)-α-phenethylamine at room temperature over 5 minutes or more, and the mixture was stirred for 20 minutes. Precipitated salt was collected by filtration and washed twice with ethyl acetate, and the washed salt was made acidic with a saturated aqueous citric acid solution. To the solution was added chloroform, the mixture was separated, and the organic layer was washed with saturated brine, dried over anhydrous magnesium sulfate, and concentrated under reduced pressure. The residue was dissolved in 1000 ml of ethyl acetate, to the solution was added 100 ml of an ethyl acetate solution containing 6.12 g of (S)-α-phenethylamine at room temperature over minutes or more, and the mixture was stirred for 15 minutes. Precipitated salt was collected by filtration and washed twice with ethyl acetate, and the washed salt was made acidic with a saturated aqueous citric acid solution. To the solution was added chloroform, the mixture was separated, and the organic layer was washed with saturated brine, dried over anhydrous magnesium sulfate, and concentrated under reduced pressure to give 14.9 g of (a) (3S,4S)-1-tert-butoxycarbonyl-4-carboxyl-3-(4-fluoro-2-methylphenyl)piperidine shown in the following Table 68. Moreover, the mother liquor obtained by the above-mentioned operation was made acidic with an aqueous citric acid solution. To the solution was added chloroform, the mixture was separated, and the organic layer was washed with saturated brine, dried over anhydrous magnesium sulfate, and concentrated under reduced pressure. The residue was dissolved in 800 ml of ethyl acetate, to the solution was added 100 ml of an ethyl acetate solution containing 7.27 g of (R)-α-phenethylamine at room temperature over 5 minutes or more, and the mixture was stirred for 20 minutes. Precipitated salt was collected by filtration and washed twice with ethyl acetate, and the washed salt was made acidic with a saturated aqueous citric acid solution. To solution was added chloroform, the mixture was separated, and the organic layer was washed with a saturated brine, dried over anhydrous magnesium sulfate, and concentrated under reduced pressure to give 17.4 g of (b) (3R,4R)-1-tert-butoxycarbonyl-4-carboxyl-3-(4-fluoro-2-methylphenyl)piperidine shown in the following Table 68. Incidentally, optical purities of the obtained (3S,4S)-1-tert-butoxycarbonyl-4-carboxyl-3-(4-fluoro-2-methylphenyl)piperidine and (3R,4R)-1-tert-butoxycarbonyl-4-carboxyl-3-(4-fluoro-2-methylphenyl)piperidine were each 99.0% ee and 94.8% ee. (6) To 150 ml of a toluene solution containing 5 g of (3S,4S)-1-tert-butoxycarbonyl-4-carboxyl-3-(4-fluoro-2-methylphenyl)piperidine obtained in the above-mentioned (5) cooled to 0° C. were added 4.49 g of diphenylphosphoric azide and 1.80 g of triethylamine, and the mixture was stirred at room temperature for 1 hour, and then, at 80° C. for 2 hours. The reaction mixture was cooled to 0° C., then, 3.53 g of benzyl alcohol and 90 mg of dimethylaminopyridine were added to the mixture, and the resulting mixture was stirred at 80° C. for 16 hours. To the reaction mixture were added ethyl acetate and a saturated aqueous sodium hydrogen carbonate solution, and the mixture was separated. Then, the organic layer was washed with water, dried over anhydrous sodium sulfate, and concentrated under reduced pressure. The obtained residue was purified by silica gel column chromatography (n-hexane:ethyl acetate=19:1→2:1) to give 5.23 g of (3S,4S)-4-benzyloxycarbonylamino-1-tert-butoxycarbonyl-3-(4-fluoro-2-methylphenyl)piperidine shown in the following Table 69.
(7) To 45 ml of an N,N-dimethylformamide solution containing 5.23 g of the compound obtained in the above-mentioned (6) cooled to 0° C. was added 497 mg of sodium hydride, and the mixture was stirred at 0° C. for 30 minutes. Then, 2.02 g of methyl iodide was added to the mixture, and the resulting mixture was stirred at room temperature for 1 hour. To the reaction mixture were added ethyl acetate and semi-saturated brine, and the mixture was separated, the organic layer was washed with semi-saturated brine, dried over anhydrous sodium sulfate, and concentrated under reduced pressure. The obtained residue was purified by basic silica gel column chromatography (n-hexane:ethyl acetate=19:1→2:1) to give 3.15 g of (3S,4S)-4-(N-benzyloxycarbonyl-N-methylamino)-1-tert-butoxy-carbonyl-3-(4-fluoro-2-methylphenyl)piperidine shown in the following Table 69.
(8) To 20 ml of a methanol solution containing 1.86 g of the compound obtained in the above-mentioned (7) was added 372 mg of 10% palladium carbon, and the mixture was stirred under hydrogen atmosphere at room temperature for 2 hours. The reaction mixture was filtered through membrane filter, and the filtrate was concentrated under reduced pressure to give 1.45 g of (3S,4S)-1-tert-butoxycarbonyl-3-(4-fluoro-2-methylphenyl)-4-(N-methylamino)piperidine shown in the following Table 69.

### Reference Example 4

(1) By using 2.5 g of (3R,4R)-1-tert-butoxycarbonyl-4-carboxyl-3-(4-fluoro-2-methylphenyl)piperidine, the same treatment as in Reference example 3(6) was carried out to give 2.8 g of (3R,4R)-4-benzyloxycarbonylamino-1-tert-butoxycarbonyl-3-(4-fluoro-2-methylphenyl)piperidine.
(2) By using 2.8 g of the compound obtained in the above-mentioned (1), the same treatment as in Reference example 3(7) was carried out to give 2.2 g of (3R,4R)-4-(N-benzyloxycarbonyl-N-methylamino)-1-tert-butoxycarbonyl-3-(4-fluoro-2-methylphenyl)piperidine.
(3) By using 2.2 g of the compound obtained in the above-mentioned (2), the same treatment as in Reference example 3(8) was carried out to give 1.6 g of (3R,4R)-1-tert-butoxycarbonyl-3-(4-fluoro-2-methylphenyl)-4-(N-methylamino)piperidine shown in the following Table 70.

### Reference Example 5

(1) By using 5.66 g of trans-1-tert-butoxycarbonyl-4-carboxyl-3-(4-fluoro-2-methylphenyl)piperidine, the same treatment as in Reference example 3(6) was carried out to give 5.5 g of trans-4-benzyloxycarbonylamino-1-tert-butoxycarbonyl-3-(4-fluoro-2-methylphenyl)piperidine.
(2) By using 4.63 g of the compound obtained in the above-mentioned (1), the same treatment as in Reference example 3(7) was carried out to give 4.8 g of trans-4-(N-benzyloxycarbonyl-N-methylamino)-1-tert-butoxycarbonyl-3-(4-fluoro-2-methylphenyl)piperidine.
(3) By using 4.8 g of the compound obtained in the above-mentioned (2), the same treatment as in Reference example 3(8) was carried out to give 3.2 g of trans-1-tert-butoxycarbonyl-3-(4-fluoro-2-methylphenyl)-4-(N-methylamino)piperidine shown in the following Table 70.

### Reference Example 6

(1) 1.3 g of cis-1-tert-butoxycarbonyl-3-(4-fluoro-2-methylphenyl)-4-methoxycarbonylpiperidine was dissolved in 5 ml of methanol and 5 ml of tetrahydrofuran, 5 ml of 2M aqueous sodium hydroxide solution was added to the solution, and the resulting mixture was stirred at room temperature for 16 hours. After neutralizing the mixture with 2M aqueous hydrochloric acid solution, the mixture was extracted twice with chloroform. The organic layer was dried over anhydrous sodium sulfate, and concentrated under reduced pressure. The obtained residue was dried under reduced pressure to give 560 mg of a mixture comprising (a) cis-l-tert-butoxycarbonyl-4-carboxyl-3-(4-fluoro-2-methylphenyl)piperidine, and (b) trans-1-tert-butoxycarbonyl-4-carboxyl-3-(4-fluoro-2-methylphenyl)piperidine (cis-isomer:trans-isomer=56:44).
(2) By using 2.65 g of 1-tert-butoxycarbonyl-4-carboxyl-3-(4-fluoro-2-methylphenyl)piperidine obtained in the same manner as in the above-mentioned (1), the same treatment as in Reference example 3(6) was carried out to give 2.4 g of 4-benzyloxycarbonylamino-1-tert-butoxycarbonyl-3-(4-fluoro-2-methylphenyl)piperidine.
(3) By using 2.37 g of the compound obtained in the above-mentioned (2), the same treatment as in Reference example 3(7) was carried out to give 2.2 g of 4-(N-benzyloxycarbonyl-N-methylamino)-1-tert-butoxycarbonyl-3-(4-fluoro-2-methylphenyl)piperidine.
(4) By using 2.17 g of the compound obtained in the above-mentioned (3), the same treatment as in Reference example 3(8) was carried out, and the obtained residue was purified by basic silica gel column chromatography (n-hexane:ethyl acetate=19:1→2:1) to give 500 mg of (a) cis-1-tert-butoxycarbonyl-3-(4-fluoro-2-methylphenyl)-4-(N-methylamino)piperidine shown in the following Table 70, and 570 mg of (b) trans-l-tert-butoxycarbonyl-3-(4-fluoro-2-methylphenyl)-4-(N-methylamino)piperidine shown in the following Table 71.

### Reference Example 7

By using 3.5 g of trans-4-benzyloxycarbonylamino-1-tert-butoxycarbonyl-3-(4-fluoro-2-methylphenyl)piperidine, the same treatment as in Reference example 3(8) was carried out to give 2.4 g of trans-4-amino-1-tert-butoxycarbonyl-3-(4-fluoro-2-methylphenyl)piperidine shown in the following Table 71.

### Reference Example 8

(1) To 100 ml of toluene solution containing 60 ml of 2M trimethyl aluminum-hexane solution was added dropwise 40 ml of a toluene solution containing 10.2 g of 4-ethoxycarbonylcyclohexanone at 0° C., and the mixture was stirred for 30 minutes. To the reaction mixture were added water and a saturated aqueous sodium hydrogen carbonate solution, and the mixture was separated. The organic layer was washed twice with water and once with saturated brine, dried over anhydrous magnesium sulfate, and concentrated under reduced pressure. The obtained residue was purified by silica gel column chromatography (hexane:ethyl acetate=85:15→75:25) to give 3.43 g of trans-4-ethoxycarbonyl-1-methylcyclohexanol shown in the following Table 71.
(2) To 24 ml of an ethanol solution containing 2.24 g of the compound obtained in the above-mentioned (1) were added 580 mg of sodium hydroxide and 12 ml of water, and the mixture was stirred at room temperature overnight. The reaction mixture was concentrated under reduced pressure, then made acidic with 2M aqueous hydrochloric acid solution, and extracted three times with chloroform. The organic layer was dried over anhydrous magnesium sulfate, and concentrated under reduced pressure to give 1.65 g of trans-4-carboxyl-1-methylcyclohexanol shown in the following Table 71.

### Reference Example 9

(1) To 100 ml of a dichloromethane solution containing 10.0 g of 3,5-dichlorobenzyl alcohol was added 15.3g of Phosphorus tribromide, and the mixture was stirred at room temperature for 16 hours. To the reaction mixture were added chloroform and water, the mixture was separated, and the organic layer was washed with brine, dried over anhydrous sodium sulfate, and concentrated under reduced pressure to give 13.6 g of 3,5-dichlorobenzylbromide shown in the following Table 72.
(2) To 120 ml of a tetrahydrofuran solution containing 2.6 g of Cobalt chloride cooled to 0° C. were added 1.5 g of sodium borohydride portionwise a period of 10 minutes, while carbon monoxide was blown in. After the reaction mixture was stirred at room temperature for 1 hour under carbon monoxide atmosphere, 20 ml of 5M sodium hydroxide solution was added slowly, and then, a 20 ml of a tetrahydrofuran solution containing 4.8 g of the compound obtained by the above-mentioned (1) was added a period of 10 minutes. The resulting mixture was stirred at 55° C. for 3 hours. After the reaction mixture was cooled to room temperature, precipitation was filtered off. To the filtrate was added 40 ml of water and made acidic with a 6M aqueous hydrochloric acid solution. To the reaction mixture was added ethyl acetate, the mixture was separated, and the organic layer was washed with brine, dried over anhydrous sodium sulfate, and concentrated under reduced pressure. The obtained residue was purified by silica gel column chromatography (chloroform:methanol=100:0→98:2) to give 1.6 g of 3,5-dichlorophenylacetic acid shown in the following Table 72.

### Reference Examples 10 to 12

The corresponding starting materials were used and treated in the same manner as in Reference Example 1, to give compounds shown in the following Table 72.

### Reference Examples 13 and 14

The corresponding starting materials were used and treated in the same manner as in Reference Example 2, to give compounds shown in the following Table 72.

### Reference Example 15

(1) To 50 ml of an acetic acid solution containing 2.7 g of methyl 3-(4-fuluorophenyl)isonicotinate was added 270 mg of platinum oxide to carry out hydrogen substitution 5 times repeatedly. Then, under hydrogen pressure of 101 kPa, the mixture was stirred at room temperature for 4 hours. The mixture was filtered through membrane filter, and the filtrate was concentrated under reduced pressure. To the obtained residue was added 50 ml of ethyl acetate, and made basic with a saturated aqueous sodium hydrogencarbonate solution. To the resulting mixture was added 3.0 g of di-tert-butyl dicarbonate, and the mixture was stirred at room temperature for 1 hour, and then, extracted with ethyl acetate twice. The combined organic layer was dried over anhydrous sodium sulfate, and concentrated under reduced pressure. The obtained residue was purified by silica gel column chromatography (n-hexane:ethyl acetate=6:1) to give 1.6 g of cis-1-tert-butoxycarbonyl-3-(4-fluorophenyl)-4-methoxycarbonylpiperidine shown in the following Table 73.
(2) The compound obtained by the above-mentioned (1) was used and treated in the same manner as in Reference Example 3(4), (6)-(8) to give 1.6 g of trans-4-benzyloxycarbonylamino-1-tert-butoxycarbonyl-3-(4-fluorophenyl)piperidine shown in the following Table 73.

### Reference Examples 16 and 17

The corresponding starting materials were used and treated in the same manner as in Reference Example 15(1), and Example 3(4), (6) to (8) to give compound shown in the following Table 74.

### Reference Examples 18 and 19

The corresponding starting materials were used and treated in the same manner as in Reference Example 3(7) and (8) to give compounds shown in the following Tables 74 and 75.

### Reference Example 20

To 12 ml of a methanol solution containing 1.15 g of the compound obtained in Reference Example 3(6) was added 115 mg of 10% palladium carbon, and the mixture was stirred under hydrogen atmosphere at room temperature for 2 hours. The reaction mixture was filtered through membrane filter, and the filtrate was concentrated under reduced pressure. To 13 ml of dichloromethane solution containing the obtained residue was added 31 mg of acetic acid, and the mixture was stirred at room temperature for 16 hours, and then, 658 mg of sodium triacetoxyborohydride was added and stirred at room temperature for 5 hours. To the reaction mixture were added chloroform and a saturated aqueous sodium hydrogen-carbonate solution, the mixture was separated, and the organic layer was dried over anhydrous sodium sulfate, and concentrated under reduced pressure. The obtained residue was purified by silica gel column chromatography (n-hexane:ethyl acetate=95:5→4:1) to give 0.56 g of (3S,4S)-1-tert-butoxycarbonyl-3-(4-fluoro-2-methylphenyl)-4-(N-isopropylamino)piperidine shown in the following Table 75.

### Reference Example 21

The corresponding starting materials were used and treated in the same manner as in Reference Example 20 to give compound shown in the following Table 75.

TABLE 1

| Example | | |
|---|---|---|
| No. | Structural formula | MS |
| 1 | 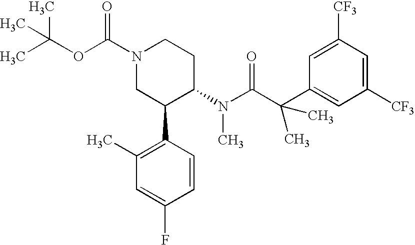 | 622 (M⁺ + 18) ESI |
| 2 | 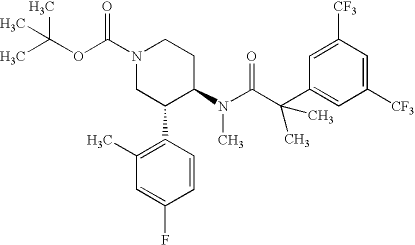 | 622 (M⁺ + 18) ESI |
| 3 | 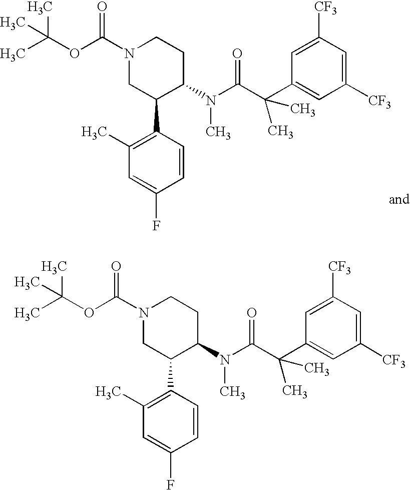 | 622 (M⁺ + 18) ESI |

TABLE 2

| Example | | |
|---|---|---|
| No. | Structural formula | MS |
| 4 | 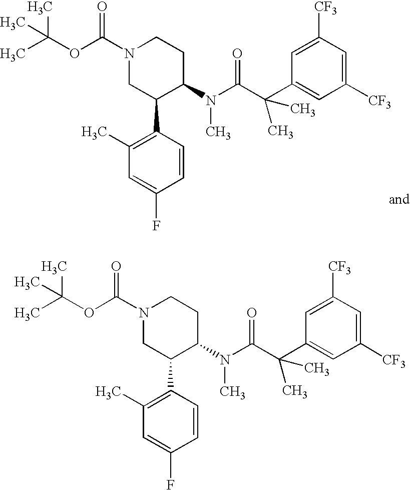 | 622 (M⁺ + 18) ESI |
| 5 | 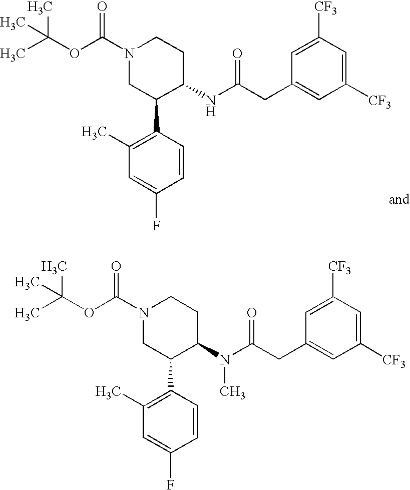 | 580 (M⁺ + 18) ESI |

TABLE 3

| Example | | |
|---|---|---|
| No. | Structural formula | MS |
| 6(a) | 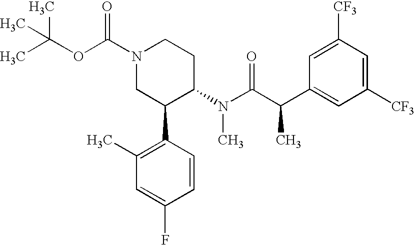 | 591(M⁺ + 1) |
| 6(b) | 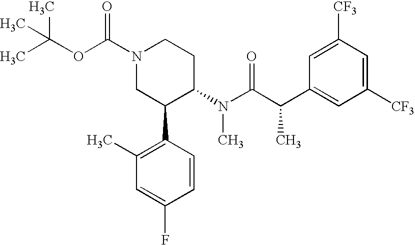 | 591(M⁺ + 1) |
| 7(a) | 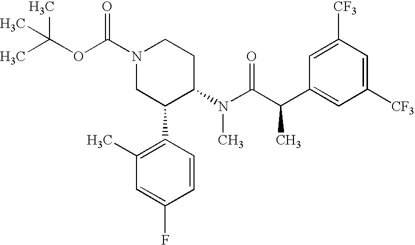 | 591(M⁺ + 1) |
| 7(b) | 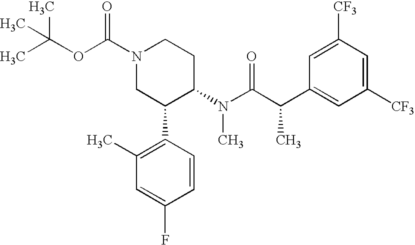 | 591(M⁺ + 1) |

TABLE 4

| Example | | |
|---|---|---|
| No. | Structural formula | MS |
| 8 | 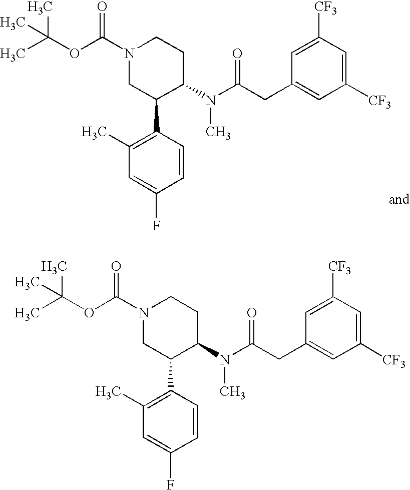 | 577(M⁺ + 1) |
| 9 | 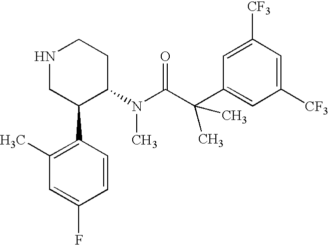 | 505(M⁺ + 1) |
| 10 | 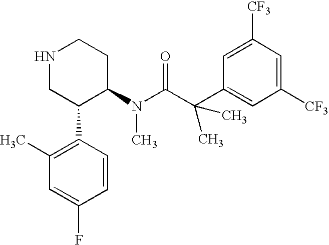 | 505(M⁺ + 1) |

TABLE 5

| Example No. | Structural formula | MS |
|---|---|---|
| 11 | 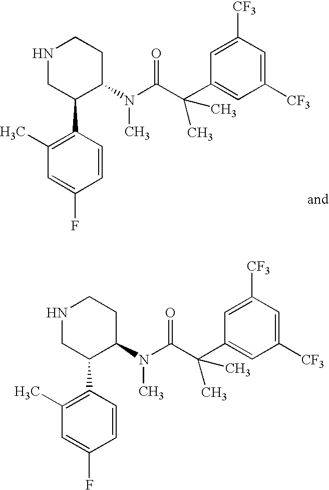 | 505(M⁺ + 1) |
| 12 | 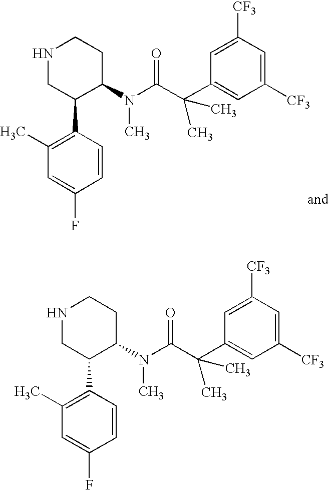 | 505(M⁺ + 1) |
| 13 | 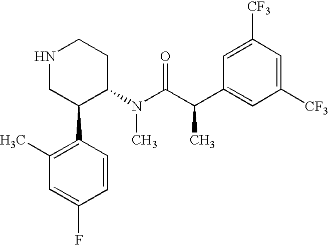 | 491(M⁺ + 1) |
| 14 | 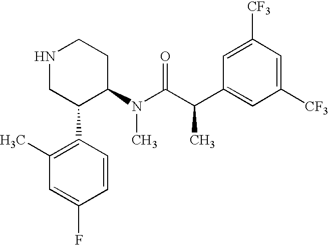 | 491(M⁺ + 1) |

TABLE 6

| Example No. | Structural formula | MS |
|---|---|---|
| 15 | 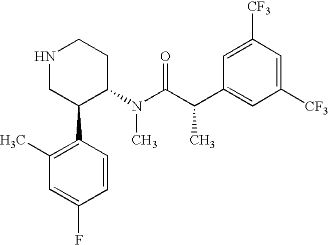 | 491(M⁺ + 1) |
| 16 | 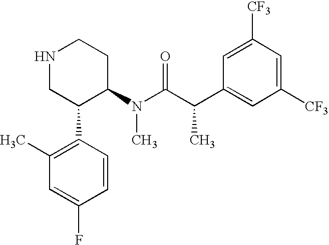 | 491(M⁺ + 1) |
| 17 | 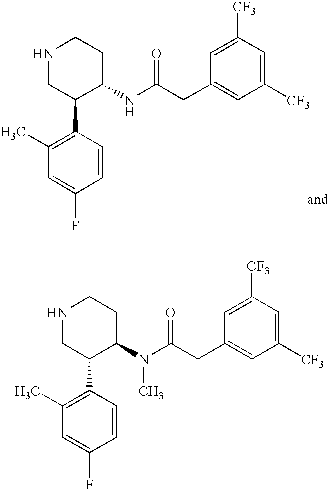 | 463(M⁺ + 1) |
| 18 | 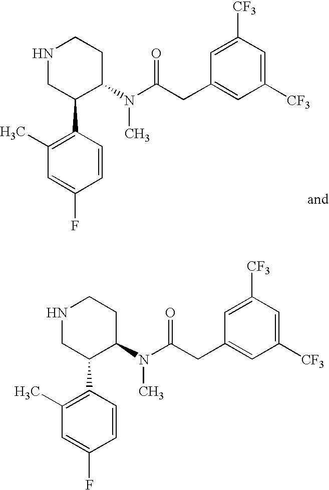 | 477(M⁺ + 1) |

TABLE 7

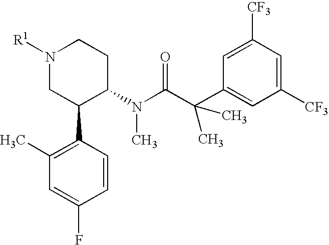

| Example No. | R¹ | MS |
|---|---|---|
| 19 | 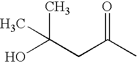 | 605(M⁺ + 1) |
| 20 | 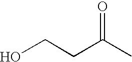 | 577(M⁺ + 1) |
| 21 | 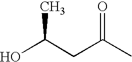 | 591(M⁺ + 1) |
| 22 | 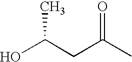 | 591(M⁺ + 1) |
| 23 | 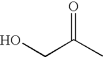 | 563(M⁺ + 1) |
| 24 | 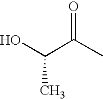 | 577(M⁺ + 1) |
| 25 | 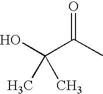 | 591(M⁺ + 1) |
| 26 | 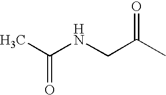 | 604(M⁺ + 1) |
| 27 | 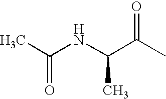 | 618(M⁺ + 1) |
| 28 | 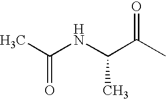 | 618(M⁺ + 1) |

TABLE 8

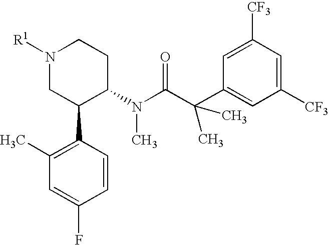

| Example No. | R¹ | MS |
|---|---|---|
| 29 | 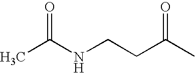 | 618(M⁺ + 1) |
| 30 | 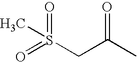 | 625(M⁺ + 1) |
| 31 | 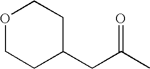 | 631(M⁺ + 1) |
| 32 | 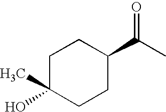 | 645(M⁺ + 1) |
| 33 | 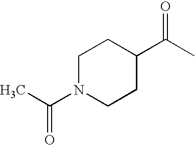 | 658(M⁺ + 1) |
| 34 | 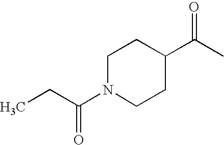 | 672(M⁺ + 1) |
| 35 | 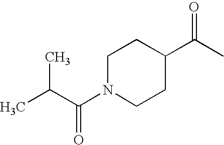 | 686(M⁺ + 1) |

TABLE 9

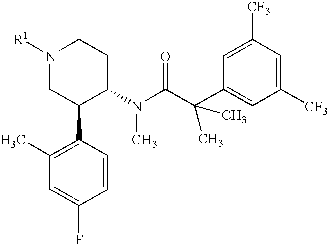

| Example No. | R¹ | MS |
|---|---|---|
| 36 | 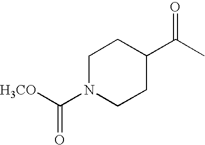 | 674(M⁺ + 1) |
| 37 | 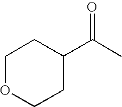 | 617(M⁺ + 1) |
| 38 | 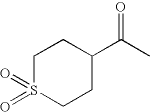 | 665(M⁺ + 1) |
| 39 | 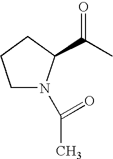 | 644(M⁺ + 1) |
| 40 | 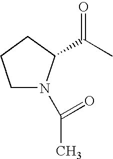 | 644(M⁺ + 1) |
| 41 | 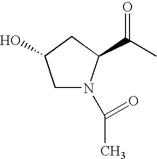 | 660(M⁺ + 1) |

TABLE 10

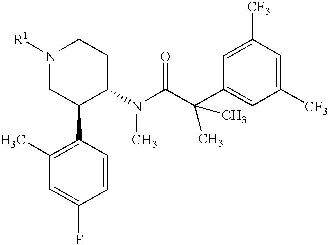

| Example No. | R¹ | MS |
|---|---|---|
| 42 | 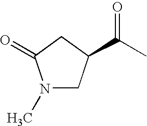 | 630(M⁺ + 1) |
| 43 | 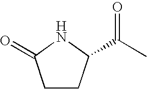 | 616(M⁺ + 1) |
| 44 | 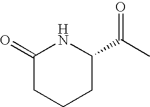 | 630(M⁺ + 1) |
| 45 | 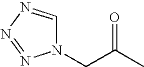 | 615(M⁺ + 1) |

TABLE 11

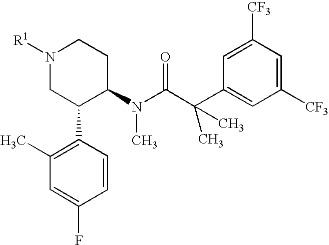

| Example No. | R¹ | MS |
|---|---|---|
| 46 | 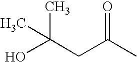 | 605 (M⁺ + 1) |
| 47 | 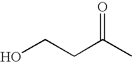 | 577 (M⁺ + 1) |
| 48 | 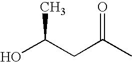 | 591 (M⁺ + 1) |
| 49 | 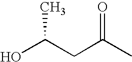 | 591 (M⁺ + 1) |
| 50 | 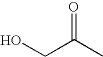 | 563 (M⁺ + 1) |
| 51 | 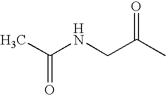 | 604 (M⁺ + 1) |
| 52 | 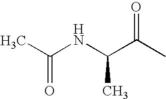 | 618 (M⁺ + 1) |
| 53 | 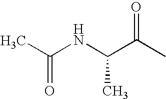 | 618 (M⁺ + 1) |
| 54 | 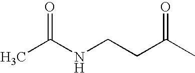 | 618 (M⁺ + 1) |
| 55 | 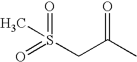 | 625 (M⁺ + 1) |

TABLE 12

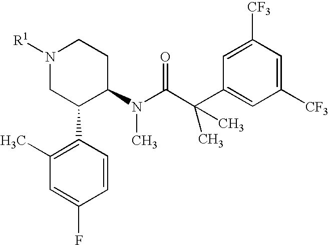

| Example No. | R¹ | MS |
|---|---|---|
| 56 | 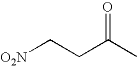 | 606 (M⁺ + 1) |
| 57 | 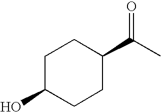 | 631 (M⁺ + 1) |
| 58 | 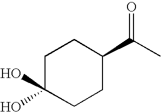 | 645 (M⁺ + 1) |
| 59 | 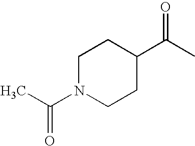 | 658 (M⁺ + 1) |
| 60 | 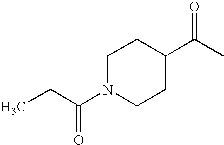 | 672 (M⁺ + 1) |
| 61 | 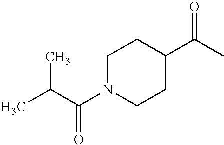 | 686 (M⁺ + 1) |
| 62 | 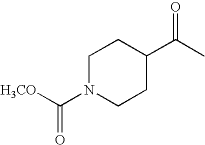 | 674 (M⁺ + 1) |

TABLE 13

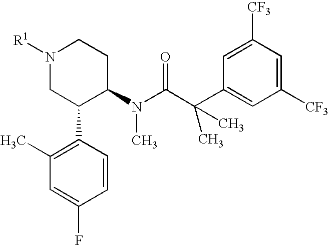

| Example No. | R¹ | MS |
|---|---|---|
| 63 | 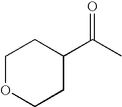 | 617 (M⁺ + 1) |
| 64 | 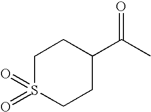 | 665 (M⁺ + 1) |
| 65 | 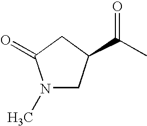 | 630 (M⁺ + 1) |
| 66 | 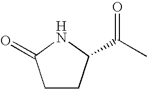 | 616 (M⁺ + 1) |
| 67 | 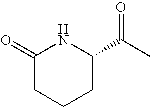 | 630 (M⁺ + 1) |

TABLE 14

| Example No. | Structural formula | MS |
|---|---|---|
| 68 | 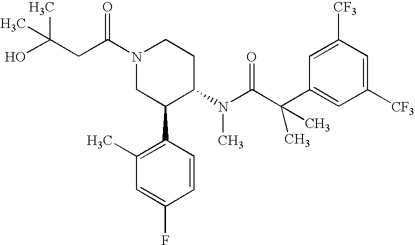 and 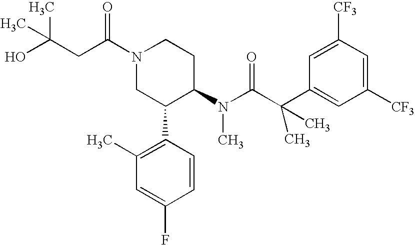 | 605 (M⁺ + 1) |
| 69 | 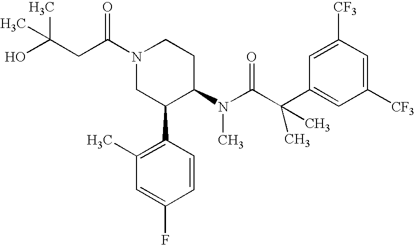 and 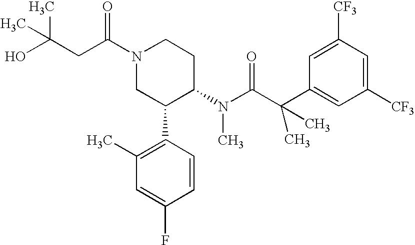 | 605 (M⁺ + 1) |

TABLE 15

| Example No. | Structural formula | MS |
|---|---|---|
| 70 | 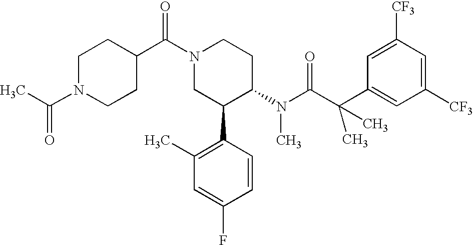 and 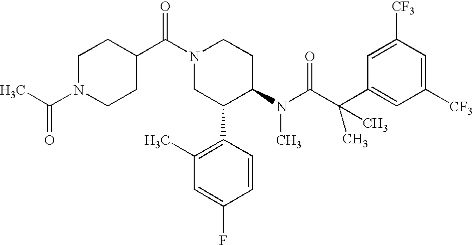 | 658 (M⁺ + 1) |
| 71 | 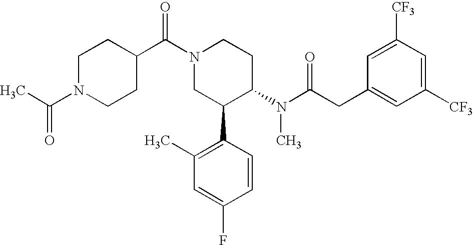 and 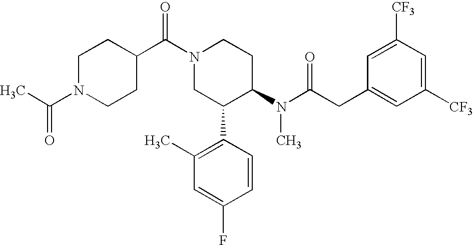 | 630 (M⁺ + 1) |

TABLE 16

| Example No. | Structural formula | MS |
|---|---|---|
| 72 | 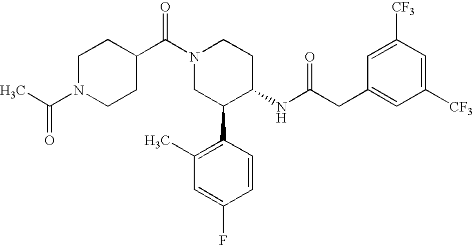 and 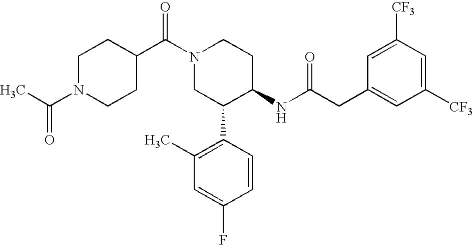 | 616 (M⁺ + 1) |
| 73 | 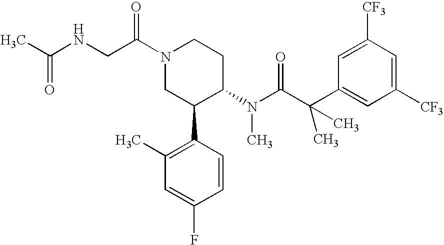 and 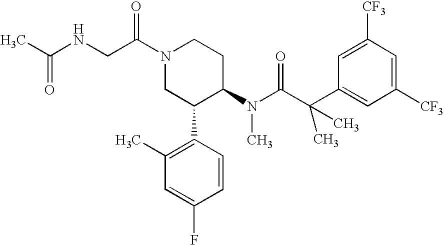 | 604 (M⁺ + 1) |

TABLE 17

| Example No. | Structural formula | MS |
|---|---|---|
| 74 | 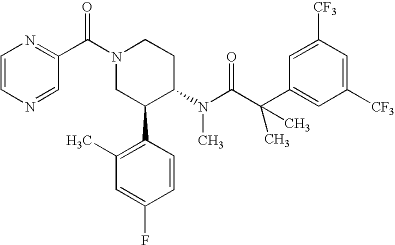 and 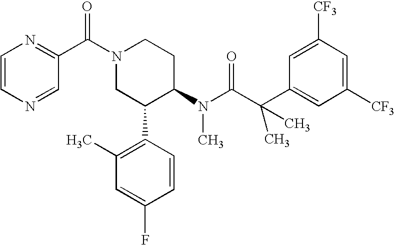 | 611 (M⁺ + 1) |

TABLE 18

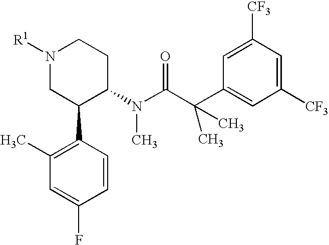

| Example No. | R¹ | MS |
|---|---|---|
| 75 | 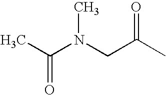 | 618 (M⁺ + 1) |
| 76 | 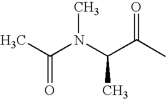 | 632 (M⁺ + 1) |
| 77 | 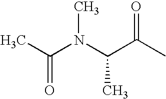 | 632 (M⁺ + 1) |
| 78 | 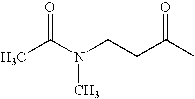 | 632 (M⁺ + 1) |
| 79 | 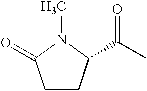 | 630 (M⁺ + 1) |
| 80 | 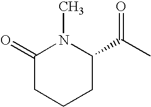 | 644 (M⁺ + 1) |

TABLE 19

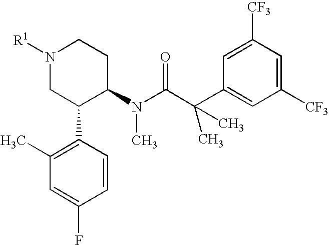

| Example No. | R¹ | MS |
|---|---|---|
| 81 | 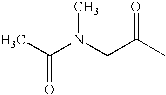 | 618 (M⁺ + 1) |
| 82 | 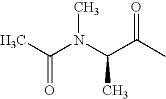 | 632 (M⁺ + 1) |
| 83 | 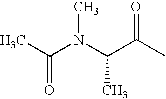 | 632 (M⁺ + 1) |
| 84 | 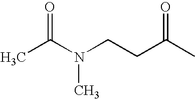 | 632 (M⁺ + 1) |
| 85 | 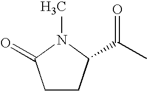 | 630 (M⁺ + 1) |
| 86 | 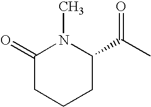 | 644 (M⁺ + 1) |

TABLE 20

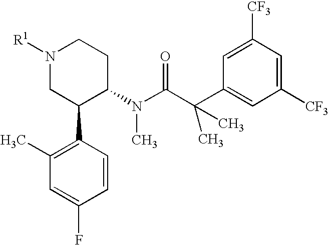

| Example No. | R¹ | MS |
|---|---|---|
| 87 | 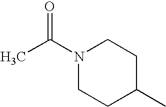 | 630 (M⁺ + 1) |
| 88 | 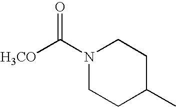 | 646 (M⁺ + 1) |
| 89 | 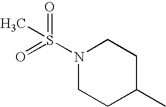 | 666 (M⁺ + 1) |
| 90 | 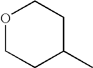 | 589 (M⁺ + 1) |
| 91 | 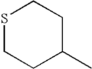 | 666 (M⁺ + 1) |
| 92 | 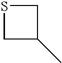 | 577 (M⁺ + 1) |
| 93 (1) | 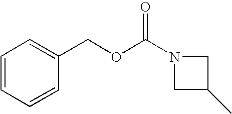 | 694 (M⁺ + 1) |
| 93 (2) | 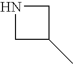 | 560 (M⁺ + 1) |

TABLE 21

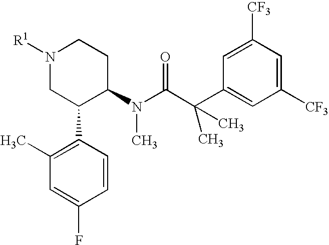

| Example No. | R¹ | MS |
|---|---|---|
| 94 | 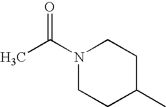 | 630 (M⁺ + 1) |
| 95 | 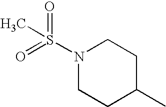 | 666 (M⁺ + 1) |
| 96 | 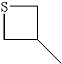 | 577 (M⁺ + 1) |
| 97 (1) | 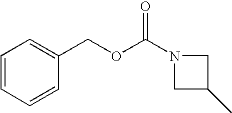 | 694 (M⁺ + 1) |
| 97 (2) | 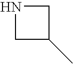 | 560 (M⁺ + 1) |

TABLE 22

| Example No. | Structural formula | MS |
|---|---|---|
| 98 | 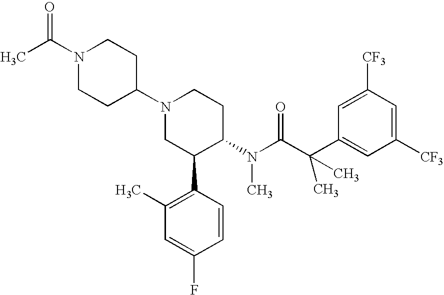 and 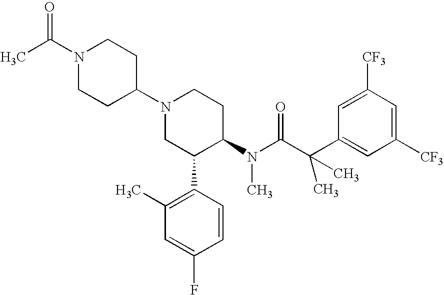 | 630 (M⁺ + 1) |
| 99 | 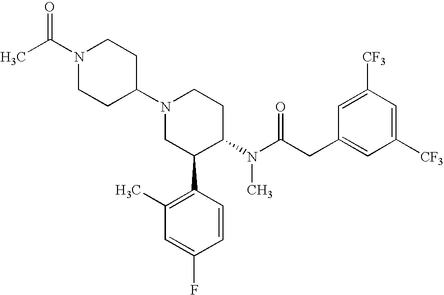 and 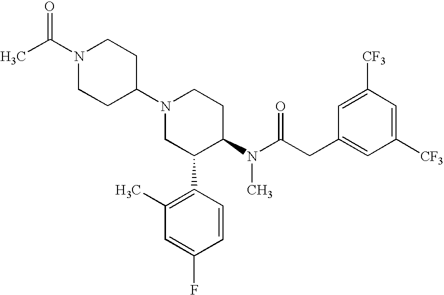 | 602 (M⁺ + 1) |

TABLE 23

| Example No. | Structural formula | MS |
|---|---|---|
| 100 | 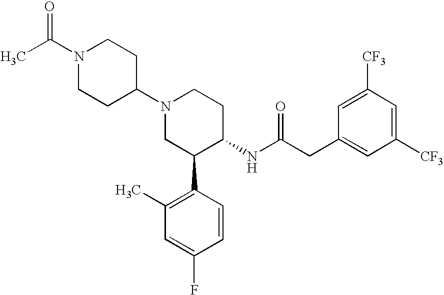 and 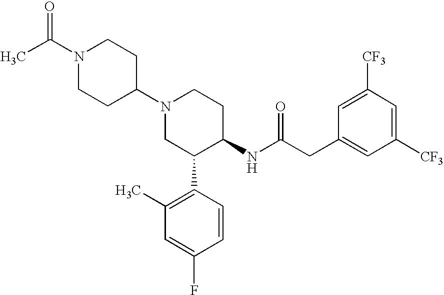 | 588 (M⁺ + 1) |

TABLE 24

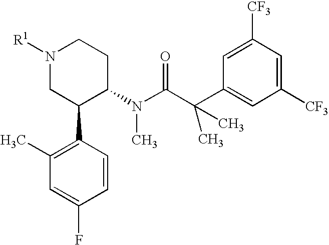

| Example No. | R¹ | MS |
|---|---|---|
| 101 | 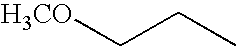 | 563 (M⁺ + 1) |
| 102 | 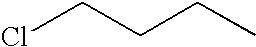 | 581, 583 (M⁺ + 1) |
| 103 | 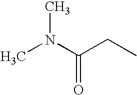 | 590 (M⁺ + 1) |
| 104 | 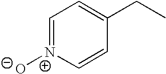 | 612 (M⁺ + 1) |
| 105 | 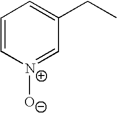 | 612 (M⁺ + 1) |
| 106 | 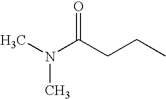 | 604 (M⁺ + 1) |
| 107 | 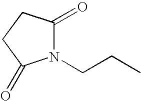 | 630 (M⁺ + 1) |

TABLE 25

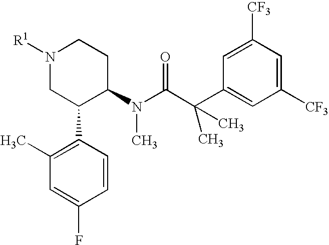

| Example No. | R¹ | MS |
|---|---|---|
| 108 | 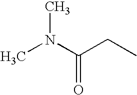 | 590 (M⁺ + 1) |
| 109 | 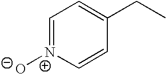 | 612 (M⁺ + 1) |
| 110 | 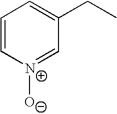 | 612 (M⁺ + 1) |
| 111 | 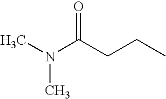 | 604 (M⁺ + 1) |

TABLE 26

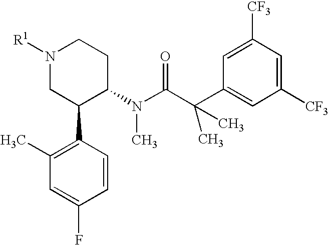

| Example No. | R¹ | MS |
|---|---|---|
| 112 | 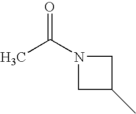 | 602 (M⁺ + 1) |
| 113 | 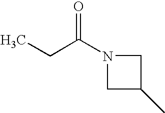 | 616 (M⁺ + 1) |
| 114 | 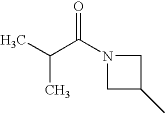 | 630 (M⁺ + 1) |
| 115 | 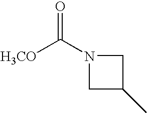 | 618 (M⁺ + 1) |
| 116 | 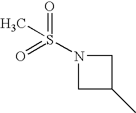 | 638 (M⁺ + 1) |
| 117 | 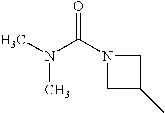 | 631 (M⁺ + 1) |
| 118 | 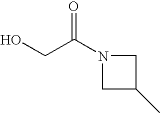 | 618 (M⁺ + 1) |

TABLE 27

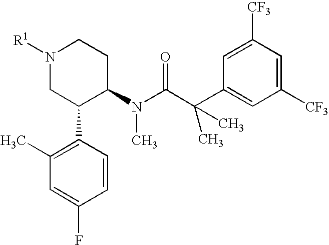

| Example No. | R¹ | MS |
|---|---|---|
| 119 | 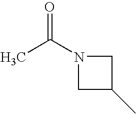 | 602 (M⁺ + 1) |
| 120 | 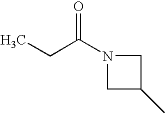 | 616 (M⁺ + 1) |
| 121 | 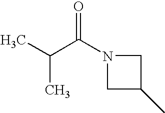 | 630 (M⁺ + 1) |
| 122 | 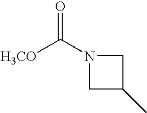 | 618 (M⁺ + 1) |
| 123 | 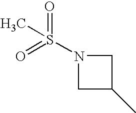 | 638 (M⁺ + 1) |
| 124 | 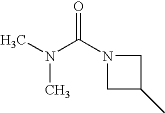 | 631 (M⁺ + 1) |
| 125 | 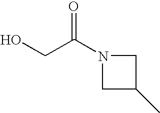 | 618 (M⁺ + 1) |

TABLE 28

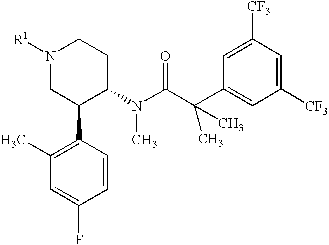

| Example No. | R¹ | MS |
|---|---|---|
| 126 | 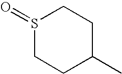 | 621 (M⁺ + 1) |
| 127 | 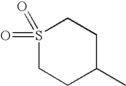 | 637 (M⁺ + 1) |
| 128 | 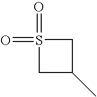 | 609 (M⁺ + 1) |

TABLE 29

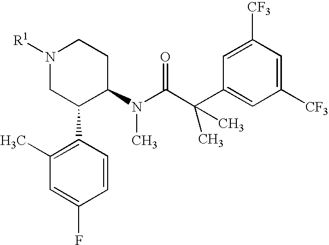

| Example No. | R¹ | MS |
|---|---|---|
| 129 | 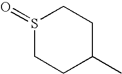 | 621 (M⁺ + 1) |
| 130 | 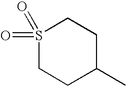 | 637 (M⁺ + 1) |
| 131 | 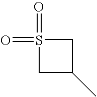 | 609 (M⁺ + 1) |

TABLE 30

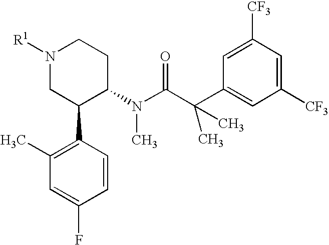

| Example No. | R¹ | MS |
|---|---|---|
| 132 | 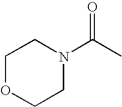 | 618 (M⁺ + 1) |
| 133 | 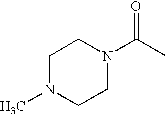 | 631 (M⁺ + 1) |
| 134 | 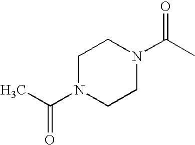 | 659 (M⁺ + 1) |
| 135 | 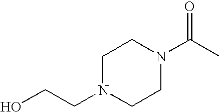 | 661 (M⁺ + 1) |
| 136 | 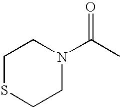 | 634 (M⁺ + 1) |
| 137 | 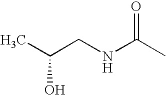 | 606 (M⁺ + 1) |
| 138 | 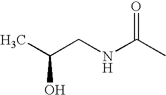 | 606 (M⁺ + 1) |
| 139 | 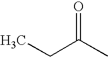 | 576 (M⁺ + 1) |

TABLE 31

| Example | | |
|---|---|---|
| No. | Structural formula | MS |
| 140 | 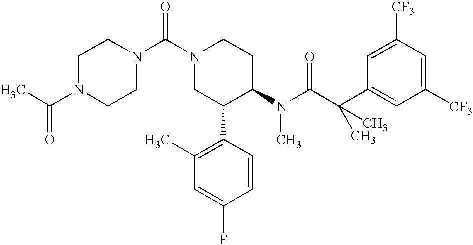 | 659 (M⁺ + 1) |
| 141 | 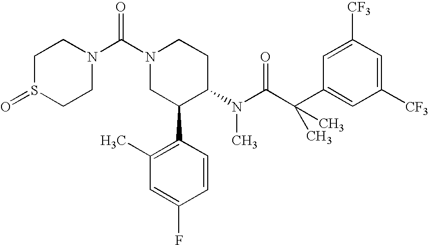 | 650 (M⁺ + 1) |
| 142 | 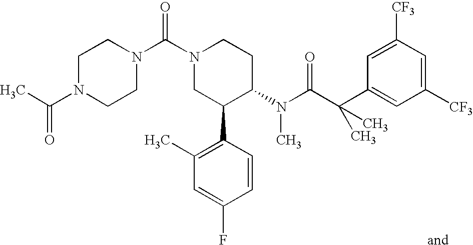 | 659 (M⁺ + 1) |
| | 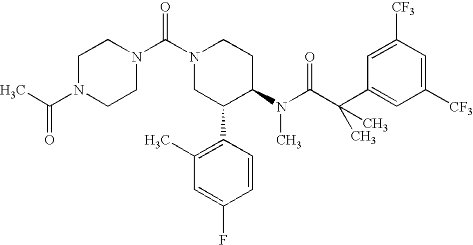 | |

TABLE 32

| Example | | |
|---|---|---|
| No. | Structural formula | MS |
| 143 | 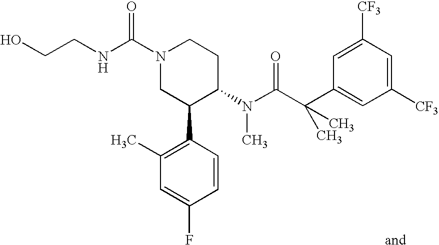 | 592 (M⁺ + 1) |
| | 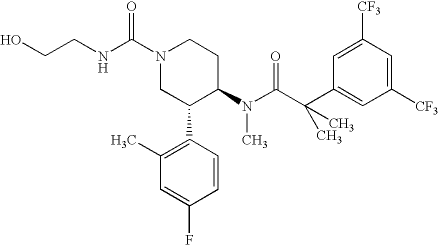 | |

TABLE 33

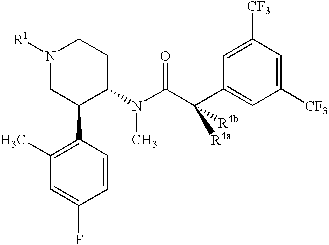

| Example | | | | |
|---|---|---|---|---|
| No. | R¹ | R^{4a} | R^{4b} | MS |
| 144 | 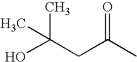 | CH₃ | H | 591 (M⁺ + 1) |
| 145 | 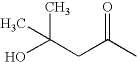 | H | CH₃ | 591 (M⁺ + 1) |
| 146 | 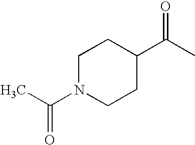 | CH₃ | H | 644 (M⁺ + 1) |
| 147 | 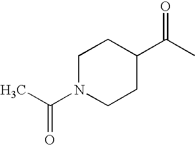 | H | CH₃ | 644 (M⁺ + 1) |
| 148 | 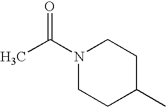 | CH₃ | H | 616 (M⁺ + 1) |
| 149 | 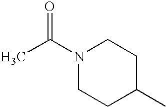 | H | CH₃ | 616 (M⁺ + 1) |

TABLE 34

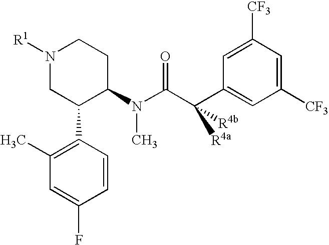

| Example | | | | |
|---|---|---|---|---|
| No. | R¹ | R^{4a} | R^{4b} | MS |
| 150 | 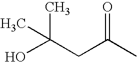 | CH₃ | H | 591 (M⁺ + 1) |
| 151 | 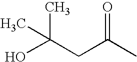 | H | CH₃ | 591 (M⁺ + 1) |
| 152 | 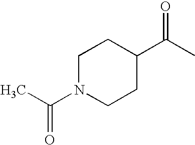 | CH₃ | H | 644 (M⁺ + 1) |
| 153 | 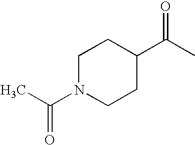 | H | CH₃ | 644 (M⁺ + 1) |
| 154 | 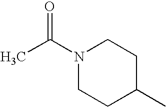 | CH₃ | H | 616 (M⁺ + 1) |
| 155 | 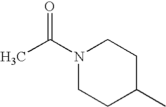 | H | CH₃ | 616 (M⁺ + 1) |

TABLE 35

| Example | | |
|---|---|---|
| No. | Structural formula | MS |
| 156 | 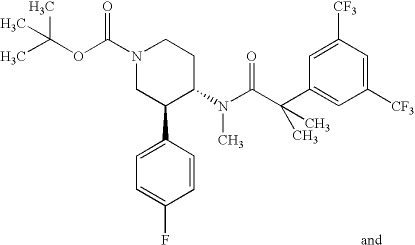 | 491 (M⁺ + 2-Boc) |
| | 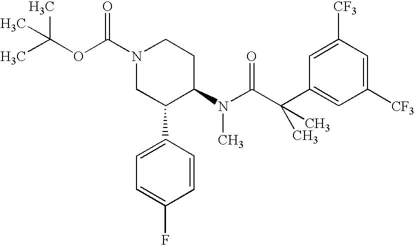 | |
| 157 | 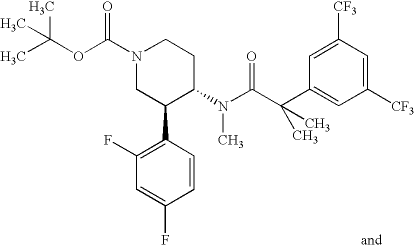 | 509 (M⁺ + 2-Boc) |
| | 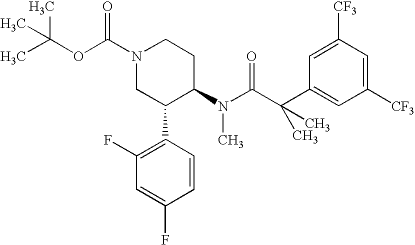 | |

The “Boc” represents tert-butoxycarbonyl moiety.

TABLE 36

| Example | | |
|---|---|---|
| No. | Structural formula | MS |
| 158 | 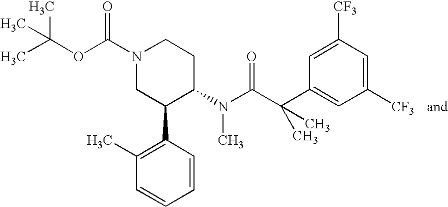 | 487 (M⁺ + 2-Boc) |
| | 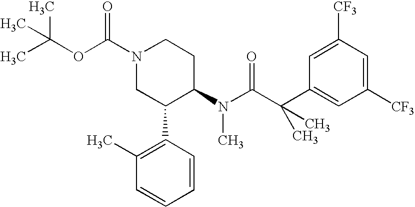 | |
| 159 | 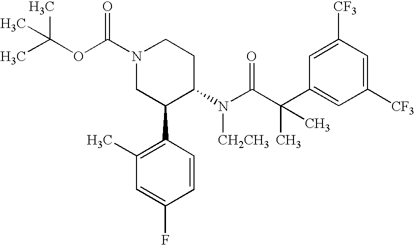 | 519 (M⁺ + 2-Boc) |
| 160 | 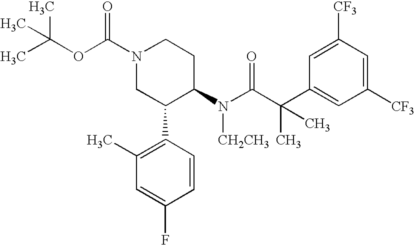 | 519 (M⁺ + 2-Boc) |
| 161 | 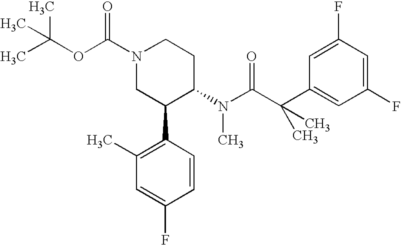 | 449 (M⁺ + 2-tert-Bu) |

The “Boc” represents tert-butoxycarbonyl moiety, and the “Bu” represents butyl moiety.

TABLE 37

| Example | | |
|---|---|---|
| No. | Structural formula | MS |
| 162 | 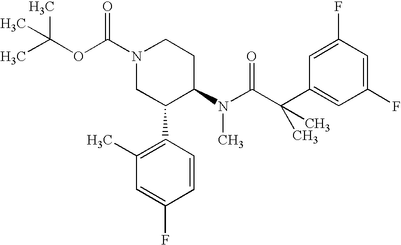 | 449 (M⁺ + 2-tert-Bu) |
| 163 | 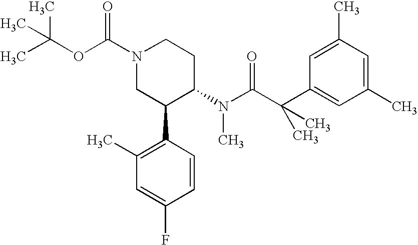 | 497 (M⁺ + 1) |
| 164 | 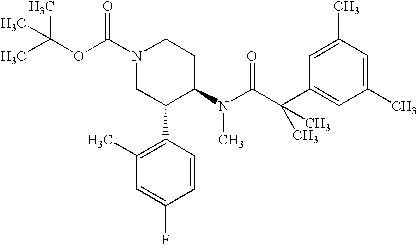 | 497 (M⁺ + 1) |
| 165 | 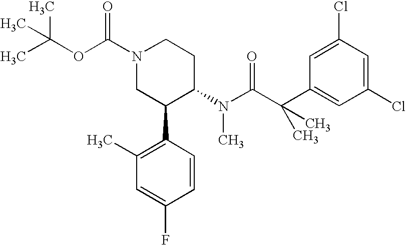 | 537/539 (M⁺ + 1) |

The “Bu” represents butyl moiety.

TABLE 38

| Example | | |
|---|---|---|
| No. | Structural formula | MS |
| 166 | 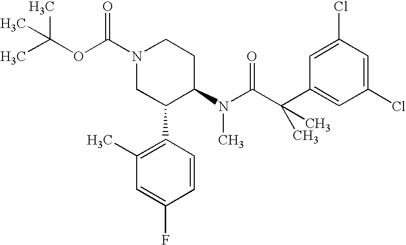 | 537/539 (M⁺ + 1) |
| 167 | 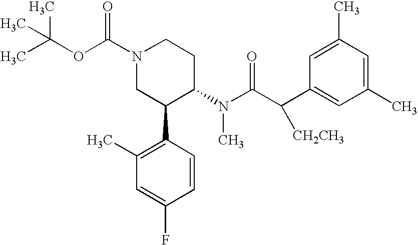 | 505 (M⁺ + 2-Boc) |
| 168 | 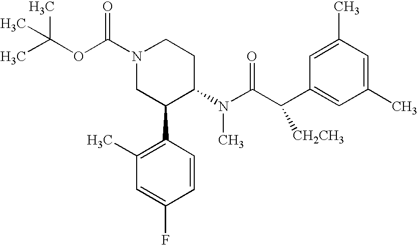 | 505 (M⁺ + 2-Boc) |
| 169 | 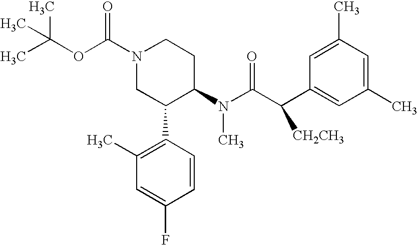 | 505 (M⁺ + 2-Boc) |

The “Boc” represents tert-butoxycarbonyl moiety.

TABLE 39

| Example | | |
|---|---|---|
| No. | Structural formula | MS |
| 170 | 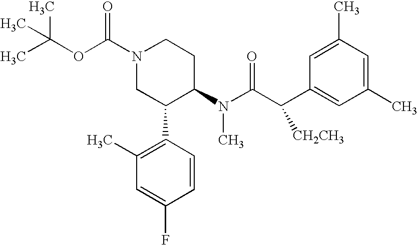 | 505 (M⁺ + 2-Boc) |
| 171 | 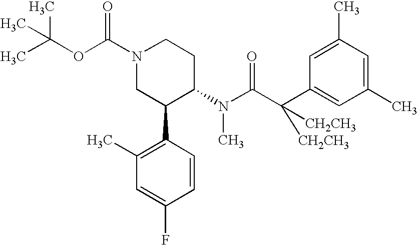 | 533 (M⁺ + 2-Boc) |
| 172 | 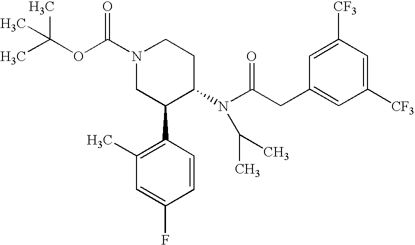 | 605 (M⁺ + 1) |
| 173 | 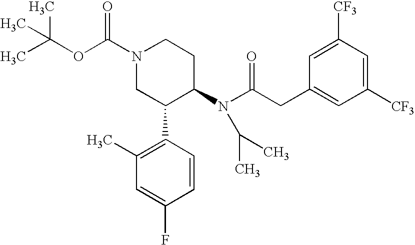 | 605 (M⁺ + 1) |

The “Boc” represents tert-butoxycarbonyl moiety.

TABLE 40

| Example | | |
|---|---|---|
| No. | Structural formula | MS |
| 174 | 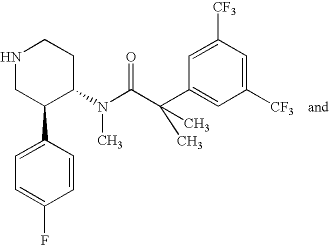 | 491 (M⁺ + 1) |
| | 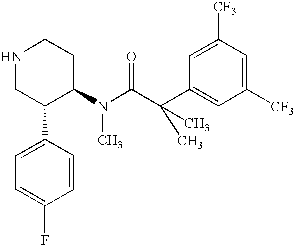 | |
| 175 | 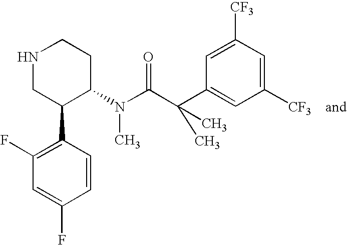 | 509 (M⁺ + 1) |
| | 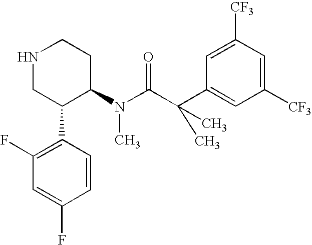 | |

TABLE 41

| Example No. | Structural formula | MS |
|---|---|---|
| 176 | 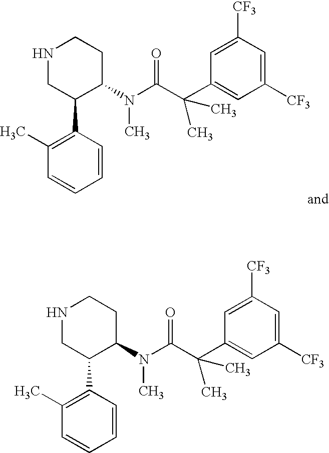 | 487(M⁺ + 1) |
| 177 | 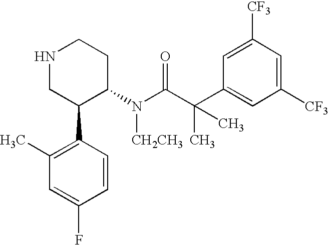 | 519(M⁺ + 1) |
| 178 | 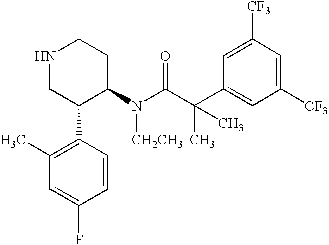 | 519(M⁺ + 1) |
| 179 | 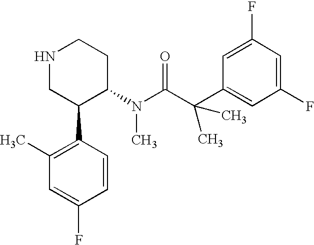 | 405(M⁺ + 1) |

TABLE 42

| Example No. | Structural formula | MS |
|---|---|---|
| 180 | 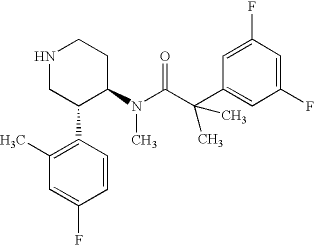 | 405(M⁺ + 1) |
| 181 | 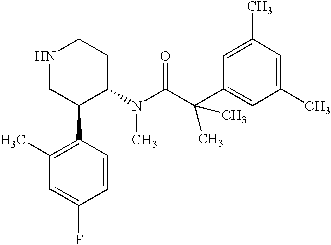 | 397(M⁺ + 1) |
| 182 | 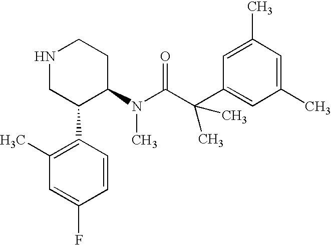 | 397(M⁺ + 1) |
| 183 | 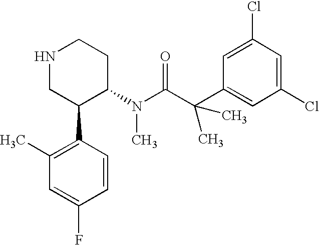 | 437/439(M⁺ + 1) |
| 184 | 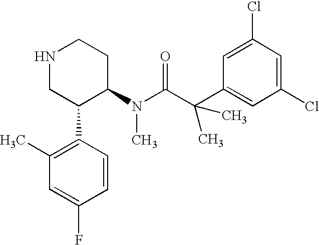 | 437/439(M⁺ + 1) |

TABLE 43

| Example No. | Structural formula | MS |
|---|---|---|
| 185 | 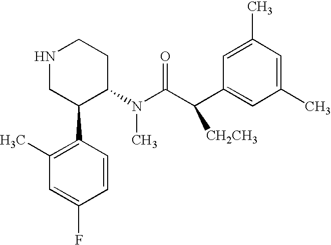 | 505(M⁺ + 1) |
| 186 | 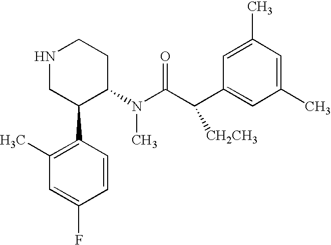 | 505(M⁺ + 1) |
| 187 | 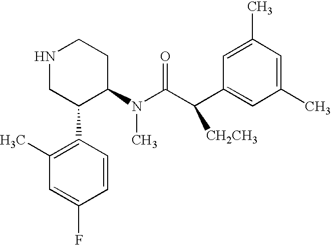 | 505(M⁺ + 1) |
| 188 | 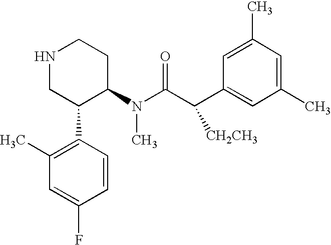 | 505(M⁺ + 1) |
| 189 | 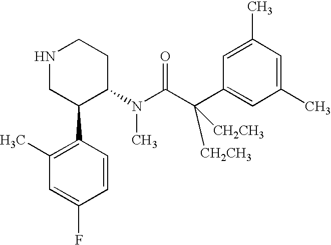 | 533(M⁺ + 1) |

TABLE 44

| Example No. | Structural formula | MS |
|---|---|---|
| 190 | 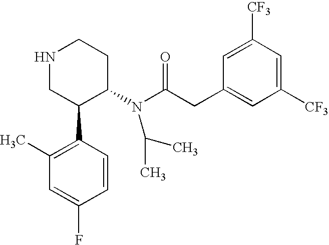 | 505(M⁺ + 1) |
| 191 | 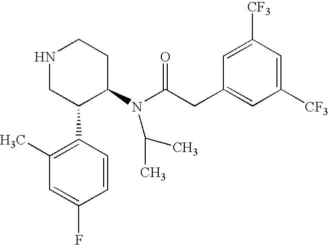 | 505(M⁺ + 1) |

TABLE 45

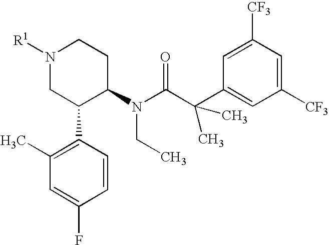

| Example No. | R¹ | MS |
|---|---|---|
| 192 | 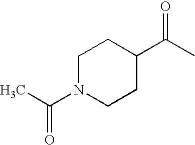 | 672(M⁺ + 1) |
| 193 | 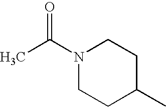 | 644(M⁺ + 1) |
| 194 | 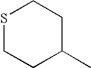 | 619(M⁺ + 1) |
| 195 | 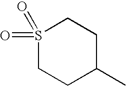 | 651(M⁺ + 1) |

TABLE 46

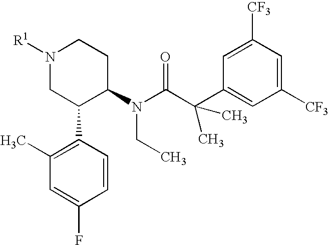

| Example No. | R¹ | MS |
|---|---|---|
| 196 | 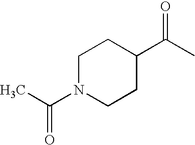 | 672(M⁺ + 1) |
| 197 | 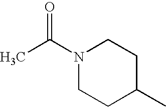 | 644(M⁺ + 1) |
| 198 | 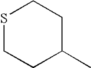 | 619(M⁺ + 1) |
| 199 | 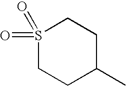 | 651(M⁺ + 1) |

TABLE 47

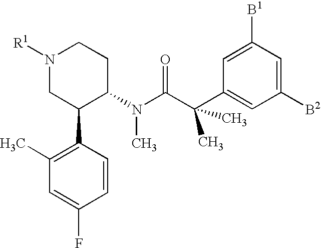

| Example | | B¹ and | |
|---|---|---|---|
| No. | R¹ | B² | MS |
| 200 | 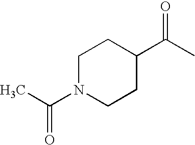 | F | 558(M⁺ + 1) |
| 201 | 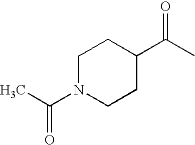 | Cl | 590/592(M⁺ + 1) |
| 202 | 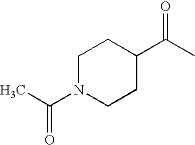 | CH₃ | 550(M⁺ + 1) |
| 203 | 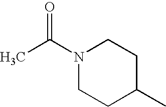 | F | 530(M⁺ + 1) |
| 204 | 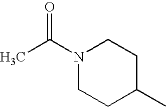 | Cl | 562/564(M⁺ + 1) |
| 205 | 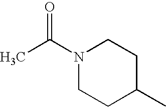 | CH₃ | 522(M⁺ + 1) |

TABLE 48

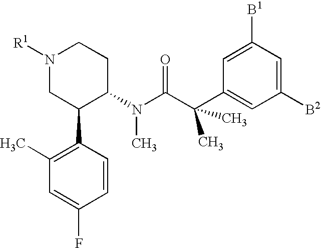

| Example | | B¹ and | |
|---|---|---|---|
| No. | R¹ | B² | MS |
| 206 | 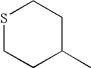 | F | 505(M⁺ + 1) |
| 207 | 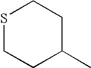 | Cl | 537/539(M⁺ + 1) |
| 208 | 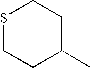 | CH₃ | 497(M⁺ + 1) |
| 209 | 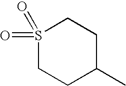 | F | 537(M⁺ + 1) |
| 210 | 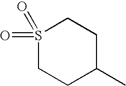 | Cl | 569/571(M⁺ + 1) |
| 211 | 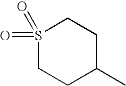 | CH₃ | 529(M⁺ + 1) |

TABLE 49

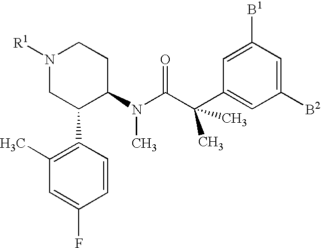

| Example | | B¹ and | |
|---|---|---|---|
| No. | R¹ | B² | MS |
| 212 | 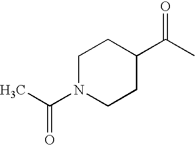 | F | 558(M⁺ + 1) |
| 213 | 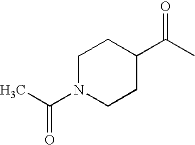 | Cl | 590/592(M⁺ + 1) |
| 214 | 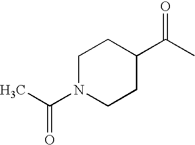 | CH₃ | 550(M⁺ + 1) |
| 215 | 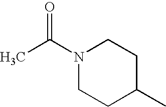 | F | 530(M⁺ + 1) |
| 216 | 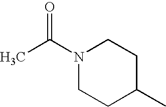 | Cl | 562/564(M⁺ + 1) |
| 217 | 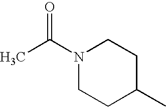 | CH₃ | 522(M⁺ + 1) |

TABLE 50

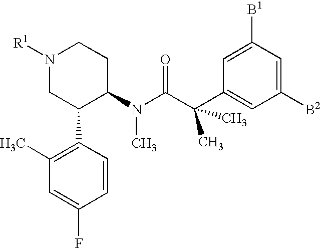

| Example | | B¹ and | |
|---|---|---|---|
| No. | R¹ | B² | MS |
| 218 | 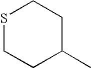 | F | 505(M⁺ + 1) |
| 219 | 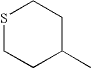 | Cl | 537/539(M⁺ + 1) |
| 220 | 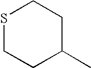 | CH₃ | 497(M⁺ + 1) |
| 221 | 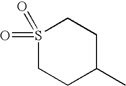 | F | 537(M⁺ + 1) |
| 222 | 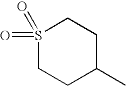 | Cl | 569/571(M⁺ + 1) |
| 223 | 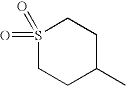 | CH₃ | 529(M⁺ + 1) |

TABLE 51

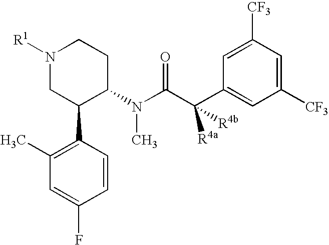

| Ex- | | | | |
|---|---|---|---|---|
| am- | | | | |
| ple | | | | |
| No. | R¹ | R^{4a} | R^{4b} | MS |
| 224 | 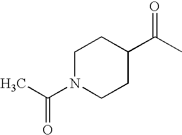 | CH₂CH₃ | H | 658 (M⁺ + 1) |
| 225 | 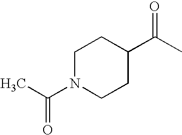 | H | CH₂CH₃ | 658 (M⁺ + 1) |
| 226 | 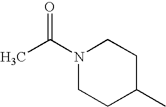 | CH₂CH₃ | H | 630 (M⁺ + 1) |
| 227 | 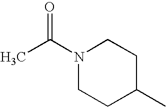 | H | CH₂CH₃ | 630 (M⁺ + 1) |
| 228 | 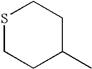 | CH₂CH₃ | H | 605 (M⁺ + 1) |
| 229 | 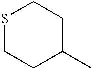 | H | CH₂CH₃ | 605 (M⁺ + 1) |
| 230 | 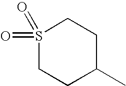 | CH₂CH₃ | H | 637 (M⁺ + 1) |
| 231 | 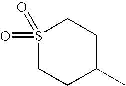 | H | CH₂CH₃ | 637 (M⁺ + 1) |

TABLE 52

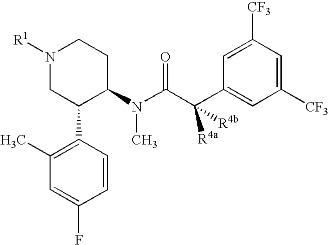

| Ex- | | | | |
|---|---|---|---|---|
| am- | | | | |
| ple | | | | |
| No. | R¹ | R^{4a} | R^{4b} | MS |
| 232 | 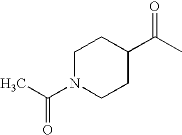 | CH₂CH₃ | H | 658 (M⁺ + 1) |
| 233 | 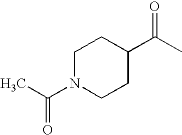 | H | CH₂CH₃ | 658 (M⁺ + 1) |
| 234 | 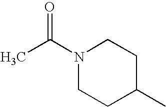 | CH₂CH₃ | H | 630 (M⁺ + 1) |
| 235 | 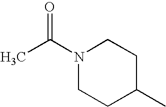 | H | CH₂CH₃ | 630 (M⁺ + 1) |
| 236 | 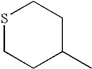 | CH₂CH₃ | H | 605 (M⁺ + 1) |
| 237 | 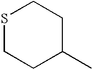 | H | CH₂CH₃ | 605 (M⁺ + 1) |
| 238 | 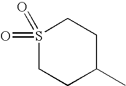 | CH₂CH₃ | H | 637 (M⁺ + 1) |
| 239 | 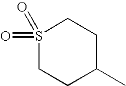 | H | CH₂CH₃ | 637 (M⁺ + 1) |

TABLE 53

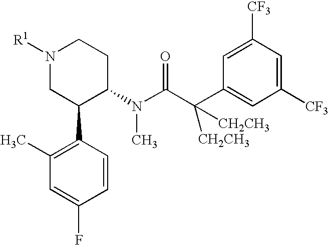

| Example No. | R¹ | MS |
|---|---|---|
| 240 | 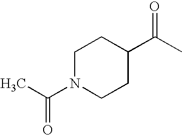 | 686 (M⁺ + 1) |
| 241 | 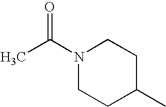 | 658 (M⁺ + 1) |
| 242 | 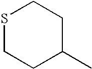 | 633 (M⁺ + 1) |
| 243 | 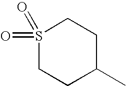 | 665 (M⁺ + 1) |

TABLE 54

| Example No. | Structural formula | MS |
|---|---|---|
| 244 | 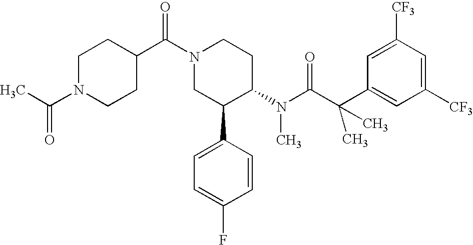 and 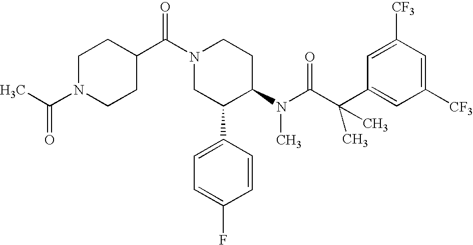 | 644 (M⁺ + 1) |
| 245 | 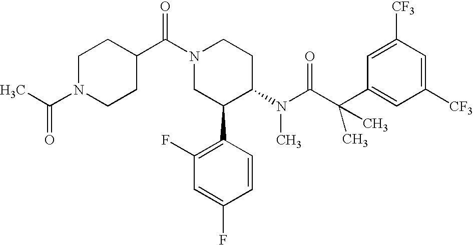 and 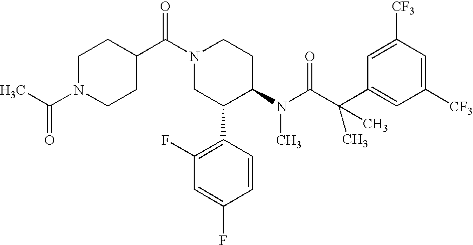 | 662 (M⁺ + 1) |

TABLE 55

| Example No. | Structural formula | MS |
|---|---|---|
| 246 | 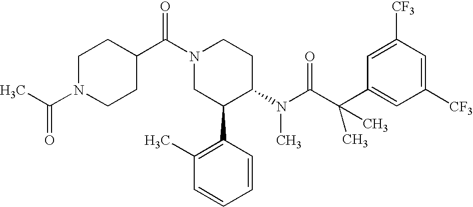 and 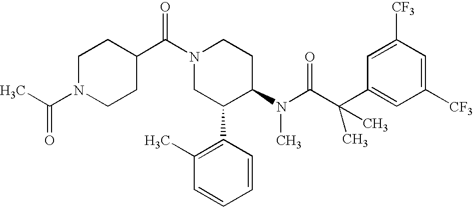 | 641 (M⁺ + 1) |
| 247 | 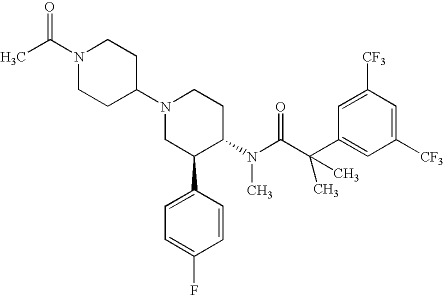 and 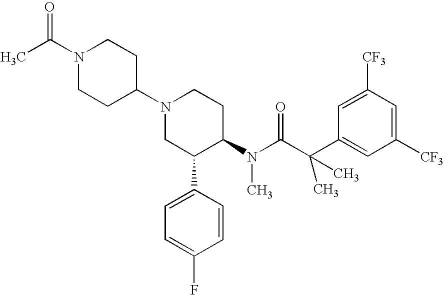 | 616 (M⁺ + 1) |

TABLE 56

| Example No. | Structural formula | MS |
|---|---|---|
| 248 | 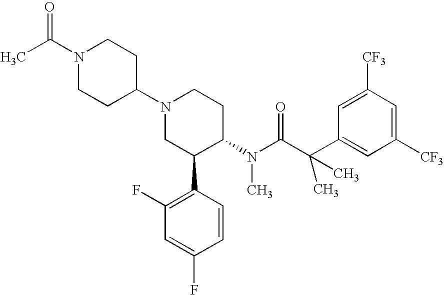 and 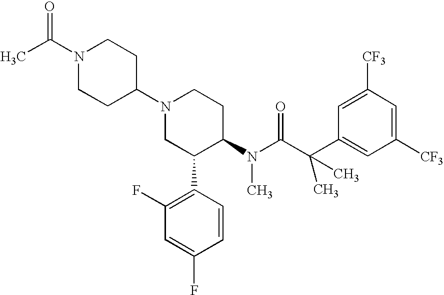 | 634 (M⁺ + 1) |
| 249 | 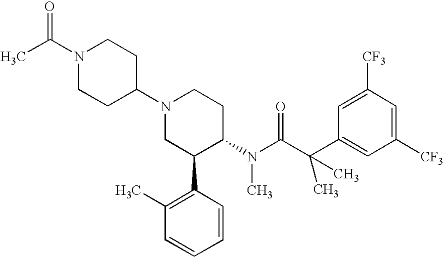 and 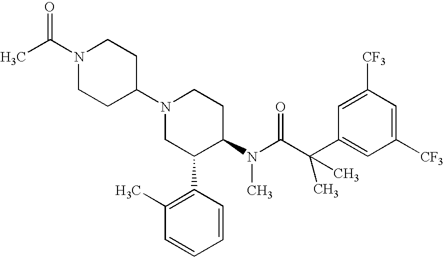 | 612 (M⁺ + 1) |

TABLE 57

| Ex- | | |
|---|---|---|
| ample | | |
| No. | Structural formula | MS |
| 250 | 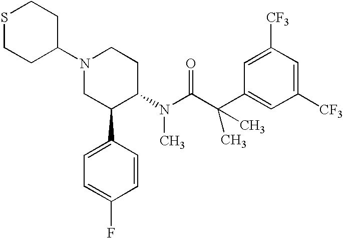 and 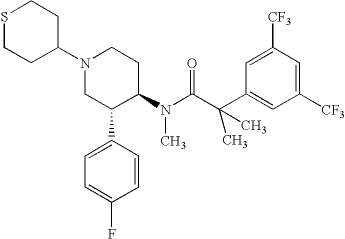 | 591 (M⁺ +1) |
| 251 | 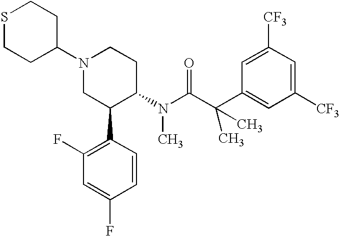 and 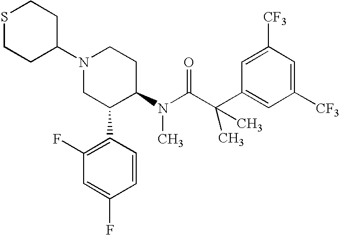 | 609 (M⁺ +1) |

TABLE 58

| Ex- | | |
|---|---|---|
| am- | | |
| ple | | |
| No. | Structural formula | MS |
| 252 | 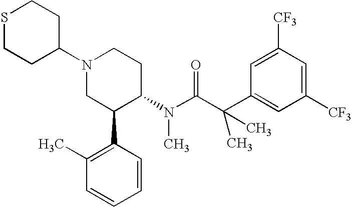 and 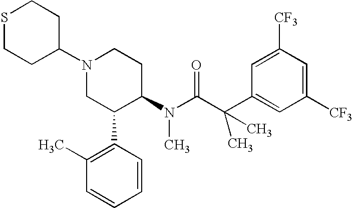 | 587 (M⁺ +1) |
| 253 | 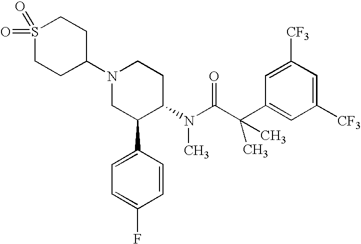 and 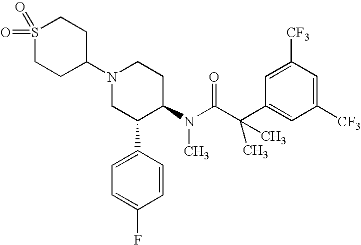 | 623 (M⁺ +1) |

TABLE 59

| Ex- | | |
|---|---|---|
| am- | | |
| ple | | |
| No. | Structural formula | MS |
| 254 | 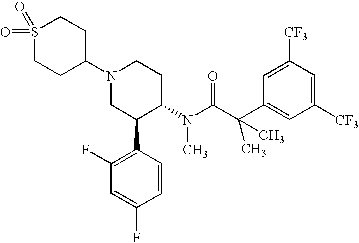 and 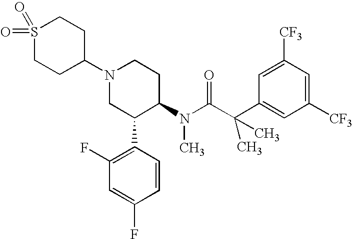 | 641 (M⁺ +1) |
| 255 | 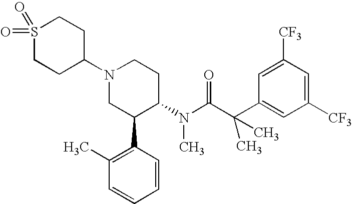 and 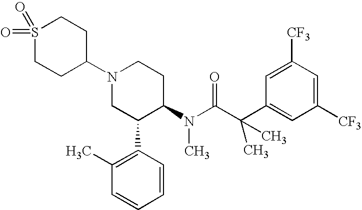 | 619 (M⁺ +1) |

TABLE 60

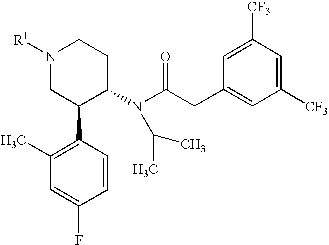

| Example No. | R¹ | MS |
|---|---|---|
| 256 | 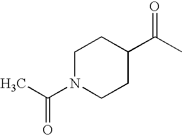 | 658 (M⁺ + 1) |
| 257 | 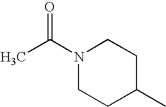 | 630 (M⁺ + 1) |
| 258 | 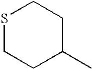 | 605 (M⁺ + 1) |
| 259 | 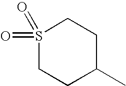 | 637 (M⁺ + 1) |

TABLE 61

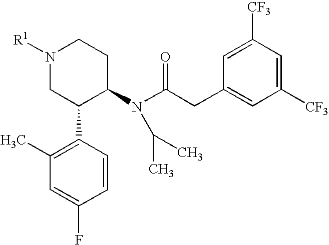

| Example No. | R¹ | MS |
|---|---|---|
| 260 | 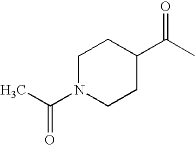 | 658 (M⁺ + 1) |
| 261 | 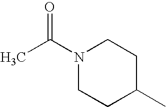 | 630 (M⁺ + 1) |

TABLE 62

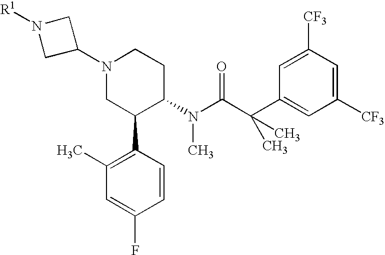

| Example No. | R¹ | MS |
|---|---|---|
| 262 | 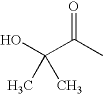 | 646 (M⁺ + 1) |
| 263 | 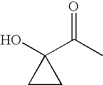 | 644 (M⁺ + 1) |
| 264 | 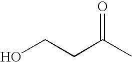 | 632 (M⁺ + 1) |
| 265 | 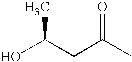 | 646 (M⁺ + 1) |
| 266 | 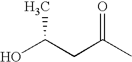 | 646 (M⁺ + 1) |
| 267 | 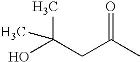 | 660 (M⁺ + 1) |
| 268 | 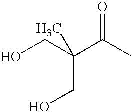 | 676 (M⁺ + 1) |
| 269 | 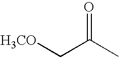 | 632 (M⁺ + 1) |
| 270 | 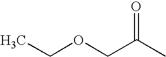 | 646 (M⁺ + 1) |
| 271 | 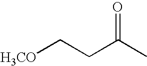 | 646 (M⁺ + 1) |

TABLE 63

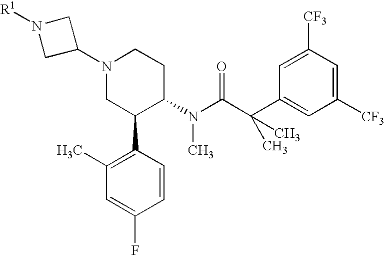

| Example No. | R¹ | MS |
|---|---|---|
| 272 | 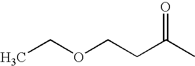 | 660 (M⁺ + 1) |
| 273 | 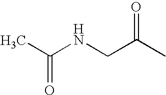 | 659 (M⁺ + 1) |
| 274 | 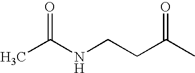 | 673 (M⁺ + 1) |
| 275 | 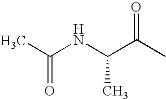 | 673 (M⁺ + 1) |
| 276 | 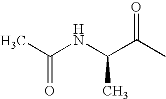 | 673 (M⁺ + 1) |
| 277 | 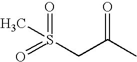 | 680 (M⁺ + 1) |
| 278 | 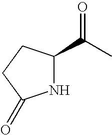 | 671 (M⁺ + 1) |
| 279 | 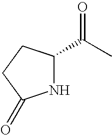 | 671 (M⁺ + 1) |

TABLE 64

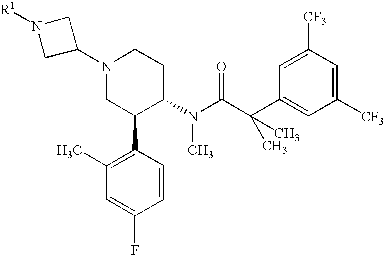

| Example No. | R¹ | MS |
|---|---|---|
| 280 | 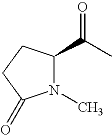 | 685 (M⁺ + 1) |
| 281 | 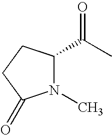 | 685 (M⁺ + 1) |
| 282 | 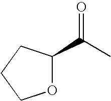 | 658 (M⁺ + 1) |
| 283 | 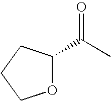 | 658 (M⁺ + 1) |
| 284 | 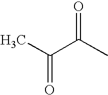 | 630 (M⁺ + 1) |
| 285 | 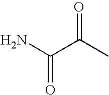 | 631 (M⁺ + 1) |

TABLE 65

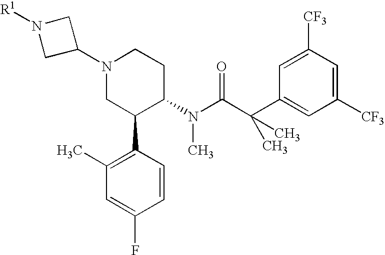

| Example No. | R¹ | MS |
|---|---|---|
| 286 | 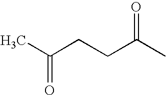 | 658 (M⁺ + 1) |
| 287 | 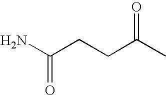 | 659 (M⁺ + 1) |
| 288 | 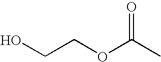 | 648 (M⁺ + 1) |
| 289 | 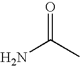 | 603 (M⁺ + 1) |
| 290 | 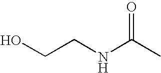 | 647 (M⁺ + 1) |
| 291 | 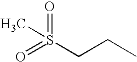 | 666 (M⁺ + 1) |
| 292 | 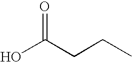 | 632 (M⁺ + 1) |

TABLE 66

| Reference | | |
|---|---|---|
| example | | |
| No. | Structural formula | MS |
| 1 (1) | 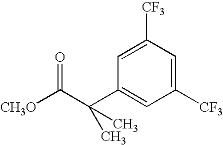 | 314 (M⁺) GC-EI |
| 1 (2) | 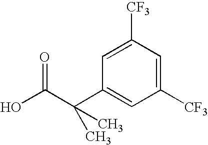 | 299 (M⁺ − 1) |
| 2 (1) | 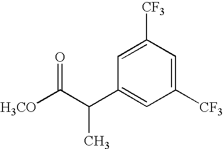 | 300 (M⁺) GC-EI |
| 2 (2) | 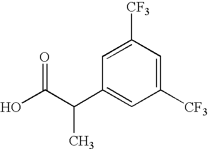 | 285 (M⁺ − 1) |
| 3 (1) | 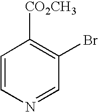 | 216, 218 (M⁺ + 1) |
| 3 (2) | 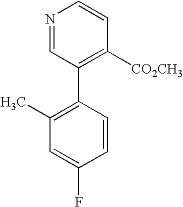 | 246 (M⁺ + 1) |

TABLE 67

| Reference | | |
|---|---|---|
| example | | |
| No. | Structural formula | MS |
| 3 (3) | 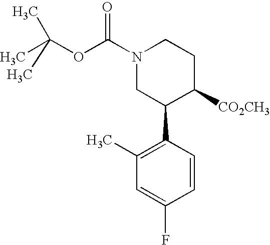 | 352 (M⁺ + 1) |
| | and | |
| | 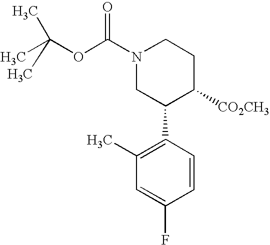 | |
| 3 (4) | 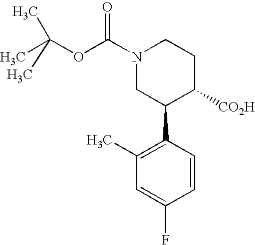 | 336 (M⁺ − 1) |
| | and | |
| | 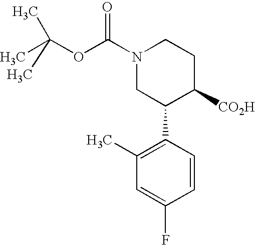 | |

TABLE 68

| Reference | | |
|---|---|---|
| example | | |
| No. | Structural formula | MS |
| 3 (5) (a) | 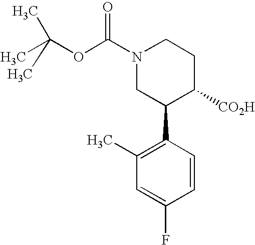 | 336 (M⁺ + 1) |
| 3 (5) (b) | 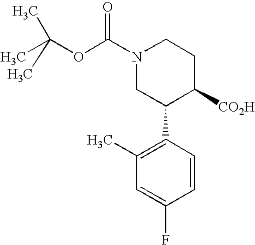 | 336 (M⁺ + 1) |

TABLE 69

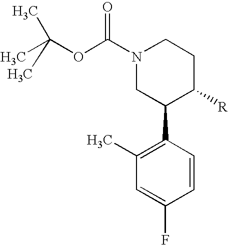

| Reference | | |
|---|---|---|
| example No. | R | MS |
| 3 (6) | 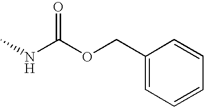 | 460 (M⁺ + 18) |
| 3 (7) | 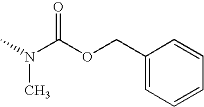 | 457 (M⁺ + 1) |
| 3 (8) | 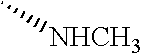 | 323 (M⁺ + 1) |

TABLE 70

| Reference | | |
|---|---|---|
| example | | |
| No. | Structural formula | MS |
| 4 (3) | 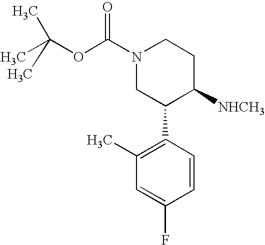 | 323 (M⁺ + 1) |
| 5 (3) | 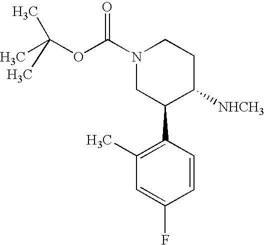 | 323 (M⁺ + 1) |
| | and | |
| | 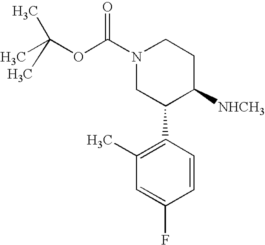 | |
| 6 (4) (a) | 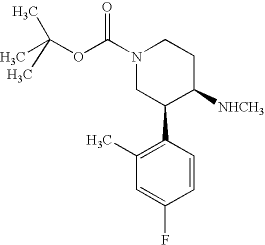 | 323 (M⁺ + 1) |
| | and | |
| | 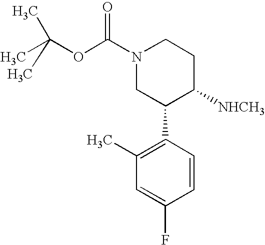 | |

TABLE 71

| Reference | | |
|---|---|---|
| example | | |
| No. | Structural formula | MS |
| 6 (4) (b) | 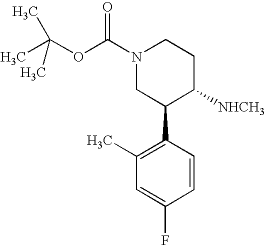 and 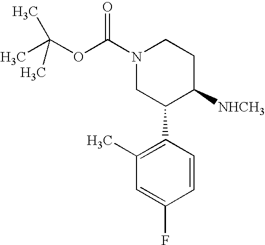 | 323 (M⁺ + 1) |
| 7 | 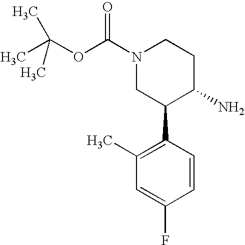 and 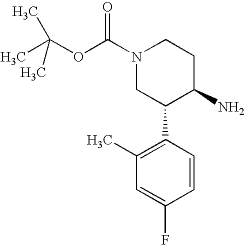 | 309 (M⁺ + 1) |
| 8 | 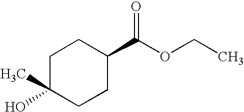 | 186 (M⁺ + 1) |
| 8 (2) | 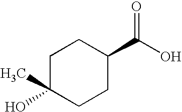 | 157 (M⁺ + 1) |

TABLE 72

| Ref- | | |
|---|---|---|
| erence | | |
| example | | |
| No. | Structural formula | MS |
| 9 (1) | 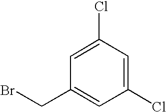 | 240 (M⁺) GC-EI |
| 9 (2) | 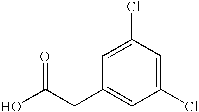 | 239/241 (M⁺ − 1 + Cl) GC-EI |
| 10 | 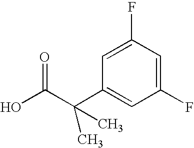 | 199 (M⁺ − 1) |
| 11 | 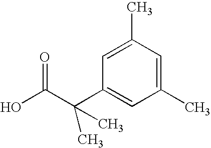 | 210 (M⁺ + 18) |
| 12 | 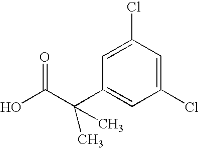 | 231/233 (M⁺ − 1) |
| 13 | 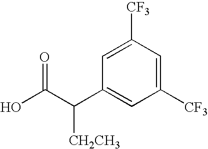 | 335/337 (M⁺ + 1 + Cl) |
| 14 | 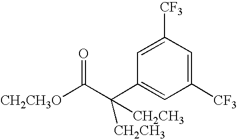 | 327 (M⁺ − 1) |

TABLE 73

| Ref- | | |
|---|---|---|
| erence | | |
| example | | |
| No. | Structural formula | MS |
| 15 (1) | 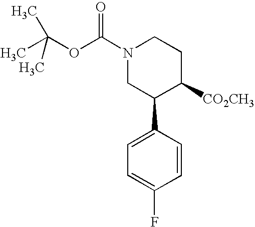 and 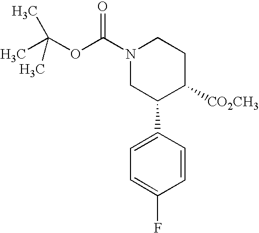 | 220 (M⁺ + 1-Boc) |
| 15 (2) | 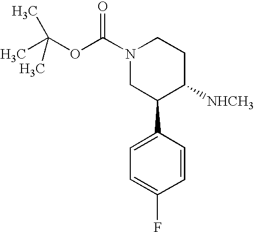 and 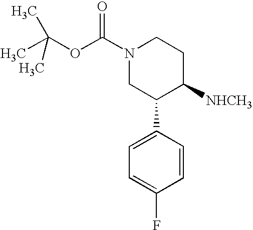 | 309 (M⁺ + 1) |

The “Boc” represents tert-butoxycabonyl moiety.

TABLE 74

| Reference | | |
|---|---|---|
| example | | |
| No. | Structural formula | MS |
| 16 | 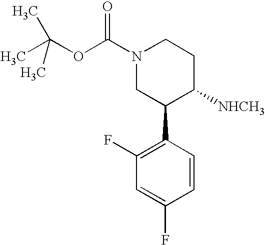 and 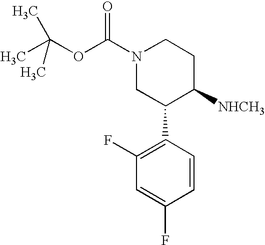 | 327 (M⁺ + 1) |
| 17 | 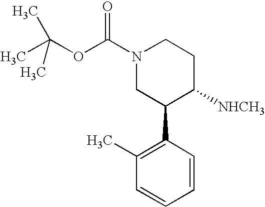 and 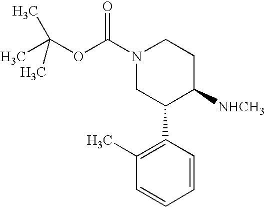 | 305 (M⁺ + 1) |
| 18 | 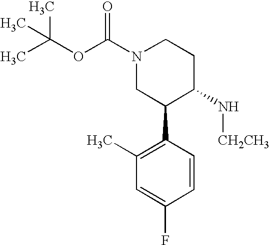 | 337 (M⁺ + 1) |

TABLE 75

| Ref- | | |
|---|---|---|
| erence | | |
| example | | |
| No. | Structural formula | MS |
| 19 | 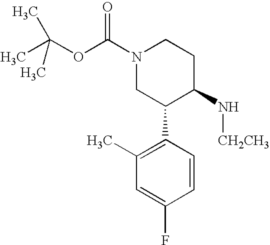 | 337 (M⁺ + 1) |
| 20 | 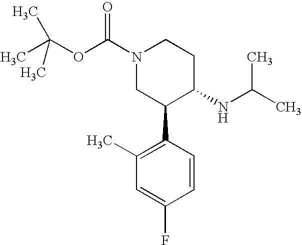 | 351 (M⁺ + 1) |
| 21 | 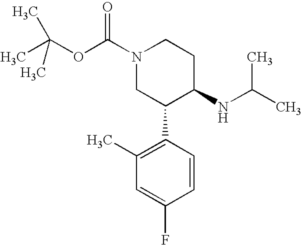 | 351 (M⁺ + 1) |

### INDUSTRIAL APPLICABILITY

The compound of the present invention or a salt thereof has an excellent tachykinin receptor antagonistic action. Further, the compound of the present invention or a salt thereof is excellent in terms of safety, absorption, penetration to the brain, metabolic stability, concentration in blood and sustainability, so that it has excellent pharmaceutical effects.

1. A piperidine compound represented by the formula [I]: 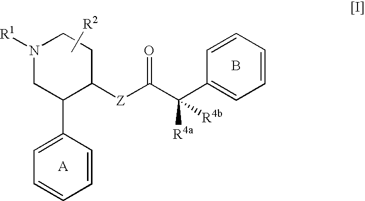
wherein Ring A represents an optionally substituted benzene ring,
Ring B represents an optionally substituted benzene ring,
R¹ represents an optionally substituted cycloalkyl group, an optionally substituted amino group or a substituted sulfonyl group,
R² represents hydrogen atom, an optionally substituted hydroxyl group, an optionally substituted amino group,
an optionally substituted alkyl group, a substituted carbonyl group or a halogen atom,
Z represents oxygen atom or a group represented by the formula: —N(R³)—,
R³ represents hydrogen atom or an optionally substituted alkyl group,
R^{4a} and R^{4b} are the same or different from each other and each is hydrogen atom or an optionally substituted alkyl group, or may be bonded to each other at the both ends to form an alkylene group,
or a pharmaceutically acceptable salt thereof.

2-5. (canceled)

6. A process for preparing a piperidine compound represented by the formula [I]: 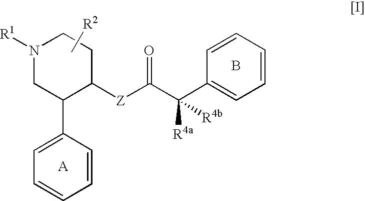
wherein
Ring A is an optionally substituted benzene ring,
Ring B is an optionally substituted benzene ring,
R¹ represents an optionally substituted cycloalkyl group, an optionally substituted amino group or a substituted sulfonyl group,
R² is hydrogen atom, an optionally substituted hydroxyl group, an optionally substituted amino group, an
optionally substituted alkyl group, a substituted carbonyl group or a halogen atom,
Z is oxygen atom or —N(R³)—,
R³ is hydrogen atom or an optionally substituted alkyl group,
R^{4a} and R^{4b} may be the same or different, and each is hydrogen atom or an optionally substituted alkyl group, or may be bonded to each other at the both ends to form an alkylene group,
or a pharmaceutically acceptable salt thereof, which comprises reacting a compound represented by the formula [II]: 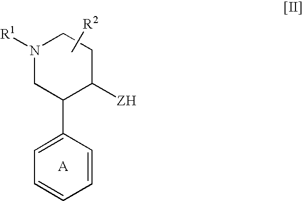
wherein Ring A, R¹, R² and Z have the same meanings as defined above,
and a compound represented by the formula [III]: 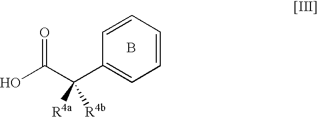
wherein Ring B, R^{4a} and R^{4b} have the same meanings as defined above,
or a reactive derivative thereof, and then, converting it into a pharmaceutically acceptable salt thereof, if necessary.

7. A pharmaceutical composition comprising the compound according to claim 1, in a clinically effective dose and a pharmaceutically acceptable carrier.

8. The compound according to claim 1 for a use as a clinically effective ingredient.

9. Use of the compound according to claim 1, for preparation of a medicament for treatment and prophylaxis of a disease selected from inflammation, allergic diseases, pain, migraine, neuralgia, itchiness, cough, central nervous system disease, digestive organs disease nausea, emesis, urinary disorder, circulatory disease and immune disorder.

10. A method for treating and preventing a disease selected from inflammation, allergic diseases, pain, migraine, neuralgia, itchiness, cough, central nervous system disease, digestive organs disease nausea, emesis, urinary disorder, circulatory disease and immune disorder, comprising administering the compound according to claim 1 in a clinically effective dose to mammal.

11. The method according to claim 10, wherein the disease is urinary disorder.
